(19) United States
(12) Patent Application Publication
(10) Pub. No.: US 2003/0236213 A1
(43) Pub. Date: Dec. 25, 2003

(54) Mimics of acyl coenzyme-A comprising pantolactone and pantothenic acid derivatives, compositions thereof, and methods of cholesterol management and related uses

(76) Inventors: Jean-Louis Dasseux, Ann Arbor, MI (US); Carmen Daniela Oniciu, Ann Arbor, MI (US)

Correspondence Address: PENNIE & EDMONDS LLP, 1667 K STREET NW, SUITE 1000, WASHINGTON, DC, 20006

(21) Appl. No.: 10/410,444

(22) Filed: Apr. 10, 2003

Related U.S. Application Data
(60) Provisional application No. 60/371,511, filed on Apr. 10, 2002.

(51) Int. Cl. A61K 48/00; A61K 31/724; A61K 31/66; C08B 37/16; C07H 21/02; A61K 31/165; A61K 31/195

(52) U.S. Cl. 514/44; 514/58; 514/114; 514/357; 514/563; 514/625; 536/23.1; 536/46; 546/336; 546/335; 558/166; 562/561; 562/899; 564/152

(57) ABSTRACT

The invention relates to novel Acyl coenzyme-A mimics, compositions comprising ketone compounds, and methods useful for treating and preventing cardiovascular diseases, dyslipidemias, dysproteinemias, and glucose metabolism disorders comprising administering a composition comprising a ketone compound. The Acyl coenzyme-A mimics, compositions, and methods of the invention are also useful for treating and preventing Alzheimer's Disease, Syndrome X, peroxisome proliferator activated receptor-related disorders, septicemia, thrombotic disorders, obesity, pancreatitis, hypertension, renal disease, cancer, inflammation, bacterial infection and impotence. In certain embodiments, the Acyl coenzyme-A mimics, compositions, and methods of the invention are useful in combination therapy with other therapeutics, such as hypocholesterolemic and hypoglycemic agents.

This application claims priority to U.S. provisional application No. 60/371,511, filed Apr. 10, 2002, the entirety of which is incorporated herein by reference.

### 1. FIELD OF THE INVENTION

The invention relates to acyl-Coenzyme-A mimics; compositions comprising an acyl coenzyme-A mimic; and methods for treating or preventing a disease or disorder, such as cardiovascular disease, dyslipidemia, dyslipoproteinemia, a disorder of glucose metabolism, Alzheimer's Disease, Syndrome X, a peroxisome proliferator activated receptor-associated disorder, septicemia, a thrombotic disorder, obesity, pancreatitis, hypertension, renal disease, cancer, inflammation, bacterial infection and impotence, comprising the administration of an acyl coenzyme-A mimic.

### 2. BACKGROUND OF THE INVENTION

Obesity, hyperlipidemia, and diabetes have been shown to play a casual role in atherosclerotic cardiovascular diseases, which currently account for a considerable proportion of morbidity in Western society. Further, one human disease, termed “Syndrome X” or “Metabolic Syndrome”, is manifested by defective glucose metabolism (insulin resistance), elevated blood pressure (hypertension), and a blood lipid imbalance (dyslipidemia). See e.g. Reaven, 1993, Annu. Rev. Med. 44:121-131.

The evidence linking elevated serum cholesterol to coronary heart disease is overwhelming. Circulating cholesterol is carried by plasma lipoproteins, which are particles of complex lipid and protein composition that transport lipids in the blood. Low density lipoprotein (LDL) and high density lipoprotein (HDL) are the major cholesterol-carrier proteins. LDL are believed to be responsible for the delivery of cholesterol from the liver, where it is synthesized or obtained from dietary sources, to extrahepatic tissues in the body. The term “reverse cholesterol transport” describes the transport of cholesterol from extrahepatic tissues to the liver, where it is catabolized and eliminated. It is believed that plasma HDL particles play a major role in the reverse transport process, acting as scavengers of tissue cholesterol. HDL is also responsible for the removal non-cholesterol lipid, oxidized cholesterol and other oxidized products from the bloodstream.

Atherosclerosis, for example, is a slowly progressive disease characterized by the accumulation of cholesterol within the arterial wall. Compelling evidence supports the belief that lipids deposited in atherosclerotic lesions are derived primarily from plasma apolipoprotein B (apo B)-containing lipoproteins, which include chylomicrons, CLDL, IDL and LDL. The apo B-containing lipoprotein, and in particular LDL, has popularly become known as the “bad” cholesterol. In contrast, HDL serum levels correlate inversely with coronary heart disease. Indeed, high serum levels of HDL is regarded as a negative risk factor. It is hypothesized that high levels of plasma HDL is not only protective against coronary artery disease, but may actually induce regression of atherosclerotic plaque (e.g., see Badimon et al., 1992, Circulation 86:(Suppl. III)86-94; Dansky and Fisher, 1999, Circulation 100:1762-3.). Thus, HDL has popularly become known as the “good” cholesterol.

### 2.1 Fatty Acid Synthesis

The first step in fatty acid synthesis is the carboxylation of acetyl coenzyme A (coA) to malonyl coA, a process catalyzed by the enzyme acetyl coA carboxylase. Malonyl coA, as well as acetyl coA, are linked to an acyl carrier protein (ACP), producing malonyl-ACP and acetyl-ACP, respectively. Malonyl-ACP and acetyl-ACP condense to form acetoactyl ACP and, following a series of reactions, butryl-ACP is formed. Fatty acid elongation proceeds by sequential addition of malonyl coA subunits (by condensation of malonyl-ACP) to butryl-ACP, and is catalyzed by an enzyme system referred to as fatty acid synthase, which in eukaryotic cells is part of a multienzyme complex. See generally Stryer, 1988, Biochemistry W. H. Freeman & Co., New York, at chapter 20.

Fatty acid synthases, also known as fatty acid ligases, are classified on the basis of the length of the carbon chain of the fatty acid to which they conjugate acetyl coA (in the form of a malonyl-ACP). Acetate-CoA ligase (EC 6.2.1.1, also known as acetyl-CoA synthetase and short chain fatty acyl-CoA synthetase) activates C2-C4 fatty acids, the butyrate-CoA ligase (EC 6.2.1.2, also known as medium chain acyl-CoA synthetase and propionoyl-CoA synthetase) activates C4-C12 while the long-chain fatty acid-CoA ligase (EC 6.2.1.3, also known as palmitoyl-CoA synthetase and long-chain acyl CoA synthetase) activates long-chain fatty acids C10-C22. Novel fatty acid syntheses are being actively identified. For example, Steinberg et al. have recently identified a human very long-chain fatty acid ligase homologous to the Drosophila “bubblegum” protein (Steinberg et al., 2000, J. Biol. Chem. 275:35162-69), and Fujino et al. have identified two murine medium-chain fatty acid ligases called MACS1 and Sa (Fujino et al., 2001, J. Biol. Chem. 276:35961-66).

### 2.2. Cholesterol Transport

The fat-transport system can be divided into two pathways: an exogenous one for cholesterol and triglycerides absorbed from the intestine and an endogenous one for cholesterol and triglycerides entering the bloodstream from the liver and other non-hepatic tissue.

In the exogenous pathway, dietary fats are packaged into lipoprotein particles called chylomicrons, which enter the bloodstream and deliver their triglycerides to adipose tissue for storage and to muscle for oxidation to supply energy. The remnant of the chylomicron, which contains cholesteryl esters, is removed from the circulation by a specific receptor found only on liver cells. This cholesterol then becomes available again for cellular metabolism or for recycling to extrahepatic tissues as plasma lipoproteins.

In the endogenous pathway, the liver secretes a large, very-low-density lipoprotein particle (VLDL) into the bloodstream. The core of VLDL consists mostly of triglycerides synthesized in the liver, with a smaller amount of cholesteryl esters either synthesized in the liver or recycled from chylomicrons. Two predominant proteins are displayed on the surface of VLDL, apolipoprotein B-100 (apo B-100) and apolipoprotein E (apo E), although other apolipoproteins are present, such as apolipoprotein CIII (apo CIII) and apolipoprotein CII (apo CII). When a VLDL reaches the capillaries of adipose tissue or of muscle, its triglyceride is extracted. This results in the formation of a new kind of particle called intermediate-density lipoprotein (IDL) or VLDL remnant, decreased in size and enriched in cholesteryl esters relative to a VLDL, but retaining its two apoproteins.

In human beings, about half of the IDL particles are removed from the circulation quickly, generally within two to six hours of their formation. This is because IDL particles bind tightly to liver cells, which extract IDL cholesterol to make new VLDL and bile acids. The IDL not taken up by the liver is catabolized by the hepatic lipase, an enzyme bound to the proteoglycan on liver cells. Apo E dissociates from IDL as it is transformed to LDL. Apo B-100 is the sole protein of LDL.

Primarily, the liver takes up and degrades circulating cholesterol to bile acids, which are the end products of cholesterol metabolism. The uptake of cholesterol-containing particles is mediated by LDL receptors, which are present in high concentrations on hepatocytes. The LDL receptor binds both apo E and apo B-100 and is responsible for binding and removing both IDL and LDL from the circulation. IN addition, remnant receptors are responsible for clearing chylomicrons and VLDL remnants i.e., IDL). However, the affinity of apo E for the LDL receptor is greater than that of apo B-100. As a result, the LDL particles have a much longer circulating life span than IDL particles; LDL circulates for an average of two and a half days before binding to the LDL receptors in the liver and other tissues. High serum levels of LDL, the “bad” cholesterol, are positively associated with coronary heart disease. For example, in atherosclerosis, cholesterol derived from circulating LDL accumulates in the walls of arteries. This accumulation forms bulky plaques that inhibit the flow of blood until a clot eventually forms, obstructing an artery and causing a heart attack or stroke.

Ultimately, the amount of intracellular cholesterol liberated from the LDL controls cellular cholesterol metabolism. The accumulation of cellular cholesterol derived from VLDL and LDL controls three processes. First, it reduces the cell's ability to make its own cholesterol by turning off the synthesis of HMGCoA reductase, a key enzyme in the cholesterol biosynthetic pathway. Second, the incoming LDL-derived cholesterol promotes storage of cholesterol by the action of ACAT, the cellular enzyme that converts cholesterol into cholesteryl esters that are deposited in storage droplets. Third, the accumulation of cholesterol within the cell drives a feedback mechanism that inhibits cellular synthesis of new LDL receptors. Cells, therefore, adjust their complement of LDL receptors so that enough cholesterol is brought in to meet their metabolic needs, without overloading (for a review, see Brown & Goldstein, In, The Pharmacological Basis Of Therapeutics, 8th Ed., Goodman & Gilman, Pergaman Press, NY, 1990, Ch. 36, pp. 874-896).

High levels of apo B-containing lipoproteins can be trapped in the subendothelial space of an artery and undergo oxidation. The oxidized lipoprotein is recognized by scavenger receptors on macrophages. Binding of oxidized lipoprotein to the scavenger receptors can enrich the macrophages with cholesterol and cholesteryl esters independently of the LDL receptor. Macrophages can also produce cholesteryl esters by the action of ACAT.

LDL can also be complexed to a high molecular weight glycoprotein called apolipoprotein(a), also known as apo(a), through a disulfide bridge. The LDL-apo(a) complex is known as Lipoprotein(a) or Lp(a). Elevated levels of Lp(a) are detrimental, having been associated with atherosclerosis, coronary heart disease, myocardial infarcation, stroke, cerebral infarction, and restenosis following angioplasty.

### 2.3. Reverse Cholesterol Transport

Peripheral (non-hepatic) cells predominantly obtain their cholesterol from a combination of local synthesis and uptake of preformed sterol from VLDL and LDL. Cells expressing scavenger receptors, such as macrophages and smooth muscle cells, can also obtain cholesterol from oxidized apo B-containing lipoproteins. In contrast, reverse cholesterol transport (RCT) is the pathway by which peripheral cell cholesterol can be returned to the liver for recycling to extrahepatic tissues, hepatic storage, or excretion into the intestine in bile. The RCT pathway represents the only means of eliminating cholesterol from most extrahepatic tissues and is crucial to maintenance of the structure and function of most cells in the body.

The enzyme in blood involved in the RCT pathway, lecithin:cholesterol acyltransferase (LCAT), converts cell-derived cholesterol to cholesteryl esters, which are sequestered in HDL destined for removal. LCAT is produced mainly in the liver and 20 circulates in plasma associated with the HDL fraction. Cholesterol ester transfer protein (CETP) and another lipid transfer protein, phospholipid transfer protein (PLTP), contribute to further remodeling the circulating HDL population (see for example Bruce et al., 1998, Annu. Rev. Nutr. 18:297-330). PLTP supplies lecithin to HDL, and CETP can move cholesteryl ester made by LCAT to other lipoproteins, particularly apoB-containing lipoproteins, such as VLDL. HDL triglyceride can be catabolized by the extracellular hepatic triglyceride lipase, and lipoprotein cholesterol is removed by the liver via several mechanisms.

Each HDL particle contains at least one molecule, and usually two to four molecules, of apolipoprotein (apo A-I). Apo A-I is synthesized by the liver and small intestine as preproapolipoprotein which is secreted as a proprotein that is rapidly cleaved to generate a mature polypeptide having 243 amino acid residues. Apo A-I consists mainly of a 22 amino acid repeating segment, spaced with helix-breaking proline residues. Apo A-I forms three types of stable structures with lipids: small, lipid-poor complexes referred to as pre-beta-1 HDL; flattened discoidal particles, referred to as pre-beta-2 HDL, which contain only polar lipids (e.g., phospholipid and cholesterol); and spherical particles containing both polar and nonpolar lipids, referred to as spherical or mature HDL (HDL₃ and HDL₂). Most HDL in the circulating population contains both apo A-I and apo A-II, a second major HDL protein. This apo A-I- and apo A-II-containing fraction is referred to herein as the AI/AII-HDL fraction of HDL. But the fraction of HDL containing only apo A-I, referred to herein as the AI-HDL fraction, appears to be more effective in RCT. Certain epidemiologic studies support the hypothesis that the AI-HDL fraction is antiartherogenic (Parra et al., 1992, Arterioscler. Thromb. 12:701-707; Decossin et al., 1997, Eur. J. Clin. Invest. 27:299-307).

Although the mechanism for cholesterol transfer from the cell surface is unknown, it is believed that the lipid-poor complex, pre-beta-1 HDL, is the preferred acceptor for cholesterol transferred from peripheral tissue involved in RCT. Cholesterol newly transferred to pre-beta-1 HDL from the cell surface rapidly appears in the discoidal pre-beta-2 HDL. PLTP may increase the rate of disc formation (Lagrost et al., 1996, J. Biol. Chem. 271:19058-19065), but data indicating a role for PLTP in RCT is lacking. LCAT reacts preferentially with discoidal and spherical HDL, transferring the 2-acyl group of lecithin or phosphatidylethanolamine to the free hydroxyl residue of fatty alcohols, particularly cholesterol, to generate cholesteryl esters (retained in the HDL) and lysolecithin. The LCAT reaction requires an apoliprotein such apo A-I or apo A-IV as an activator. ApoA-I is one of the natural cofactors for LCAT. The conversion of cholesterol to its HDL-sequestered ester prevents re-entry of cholesterol into the cell, resulting in the ultimate removal of cellular cholesterol. Cholesteryl esters in the mature HDL particles of the AI-HDL fraction are removed by the liver and processed into bile more effectively than those derived from the AI/AII-HDL fraction. This may be due, in part, to the more effective binding of AI-HDL to the hepatocyte membrane. Several HDL receptor receptors have been identified, the most well characterized of which is the scavenger receptor class B, type I (SR-BI) (Acton et al., 1996, Science 271:518-520). The SR-BI is expressed most abundantly in steroidogenic tissues (e.g., the adrenals), and in the liver (Landshulz et al., 1996, J. Clin. Invest. 98:984-995; Rigotti et al., 1996, J. Biol. Chem. 271:33545-33549). Other proposed HDL receptors include HB1 and HB2 (Hidaka and Fidge, 1992, Biochem J. 15:161-7; Kurata et al., 1998, J. Atherosclerosis and Thrombosis 4:112-7).

While there is a consensus that CETP is involved in the metabolism of VLDL- and LDL-derived lipids, its role in RCT remains controversial. However, changes in CETP activity or its acceptors, VLDL and LDL, play a role in “remodeling” the HDL population. For example, in the absence of CETP, the HDL becomes enlarged particles that are poorly removed from the circulation (for reviews on RCT and HDLs, see Fielding & Fielding, 1995, J. Lipid Res. 36:211-228; Barrans et al., 1996, Biochem. Biophys. Acta. 1300:73-85; Hirano et al., 1997, Arterioscler. Thromb. Vasc. Biol. 17:1053-1059).

### 2.4. Reverse Transport of other Lipids

HDL is not only involved in the reverse transport of cholesterol, but also plays a role in the reverse transport of other lipids, i.e., the transport of lipids from cells, organs, and tissues to the liver for catabolism and excretion. Such lipids include sphingomyelin, oxidized lipids, and lysophophatidylcholine. For example, Robins and Fasulo (1997, J. Clin. Invest. 99:380-384) have shown that HDL stimulates the transport of plant sterol by the liver into bile secretions.

### 2.5. Peroxisome Proliferator Activated Receptor Pathway

Peroxisomes are single-membrane organelles involved in β-oxidation of a number of substrates in eukaryotic cells, such as long chain fatty acids, saturated and unsaturated very long chain fatty acids, and long chain dicarboxylic acids. A structurally diverse class of compounds called peroxisome proliferators has been characterized as anti-cholesterolemic therapeutics. When administered to test rodents, peroxisome proliferators elicit dramatic increases in the size and number of hepatic and renal peroxisomes, as well as concomitant increases in the capacity of peroxisomes to metabolize fatty acids via increased expression of the enzymes required for the β-oxidation cycle (Lazarow and Fujiki, 1985, Ann. Rev. Cell Biol. 1:489-530; Vamecq and Draye, 1989, Essays Biochem. 24:1115-225; and Nelali et al., 1988, Cancer Res. 48:5316-5324). Chemicals included in this group are the fibrate class of hypolipidermic drugs, herbicides, and phthalate plasticizers (Reddy and Lalwani, 1983, Crit. Rev. Toxicol. 12:1-58). Peroxisome proliferation can also be elicited by dietary or physiological factors, such as a high-fat diet and cold acclimatization.

Insight into the mechanism whereby peroxisome proliferators exert their pleiotropic effects was provided by the identification of a member of the nuclear hormone receptor superfamily activated by these chemicals (Isseman and Green, 1990, Nature, 347:645-650). This receptor, termed peroxisome proliferator activated receptor α (PPAR_{α}), was subsequently shown to be activated by a variety of medium and long-chain fatty acids. PPAR_{α} activates transcription by binding to DNA sequence elements, termed peroxisome proliferator response elements (PPRE), in the form of a heterodimer with the retinoid X receptor (RXR). RXR is activated by 9-cis retinoic acid (see Kliewer et al., 1992, Nature 358:771-774; Gearing et al., 1993, Proc. Natl. Acad. Sci. USA 90:1440-1444, Keller et al., 1993, Proc. Natl. Acad. Sci. USA 90:2160-2164; Heyman et al., 1992, Cell 68:397-406, and Levin et al., 1992, Nature 355:359-361). Since the discovery of PPA_{α}, additional isoforms of PPAR have been identified, e.g., PPAR_{β}, PPAR_{γ} and PPAR_{δ}, which are have similar functions and are similarly regulated.

PPREs have been identified in the enhancers of a number of genes encoding proteins that regulate lipid metabolism. These proteins include the three enzymes required for peroxisomal β-oxidation of fatty acids; apolipoprotein A-I; medium-chain acyl-CoA dehydrogenase, a key enzyme in mitochondrial β-oxidation; and aP2, a lipid binding protein expressed exclusively in adipocytes (reviewed in Keller and Whali, 1993, TEM, 4:291-296; see also Staels and Auwerx, 1998, Atherosclerosis 137 Suppl:S119-23). The nature of the PPAR target genes coupled with the activation of PPARs by fatty acids and hypolipidemic drugs suggests a physiological role for the PPARs in lipid homeostasis.

Pioglitazone, an antidiabetic compound of the thiazolidinedione class, was reported to stimulate expression of a chimeric gene containing the enhancer/promoter of the lipid-binding protein aP2 upstream of the chloroamphenicol acetyl transferase reporter gene (Harris and Kletzien, 1994, Mol. Pharmacol. 45:439-445). Deletion analysis led to the identification of an approximately 30 bp region responsible for pioglitazone responsiveness. In an independent study, this 30 bp fragment was shown to contain a PPRE (Tontonoz et al.,1994, Nucleic Acids Res. 22:5628-5634). Taken together, these studies suggested the possibility that the thiazolidinediones modulate gene expression at the transcriptional level through interactions with a PPAR and reinforce the concept of the interrelatedness of glucose and lipid metabolism.

### 2.6. Current Cholesterol Management Therapies

In the past two decades or so, the segregation of cholesterolemic compounds into HDL and LDL regulators and recognition of the desirability of decreasing blood levels of the latter has led to the development of a number of drugs. However, many of these drugs have undesirable side effects and/or are contraindicated in certain patients, particularly when administered in combination with other drugs.

Bile-acid-binding resins are a class of drugs that interrupt the recycling of bile acids from the intestine to the liver. Examples of bile-acid-binding resins are cholestyramine (QUESTRAN LIGHT, Bristol-Myers Squibb), and colestipol hydrochloride (COLESTID, Pharmacia & Upjohn Company). When taken orally, these positively charged resins bind to negatively charged bile acids in the intestine. Because the resins cannot be absorbed from the intestine, they are excreted, carrying the bile acids with them. The use of such resins, however, at best only lowers serum cholesterol levels by about 20%. Moreover, their use is associated with gastrointestinal side-effects, including constipation and certain vitamin deficiencies. Moreover, since the resins bind to drugs, other oral medications must be taken at least one hour before or four to six hours subsequent to ingestion of the resin, complicating heart patients' drug regimens.

The statins are inhibitors of cholesterol synthesis. Sometimes, the statins are used in combination therapy with bile-acid-binding resins. Lovastatin (MEVACOR, Merck & Co., Inc.), a natural product derived from a strain of Aspergillus; pravastatin (PRAVACHOL, Bristol-Myers Squibb Co.); and atorvastatin (LIPITOR, Warner Lambert) block cholesterol synthesis by inhibiting HMGCoA, the key enzyme involved in the cholesterol biosynthetic pathway. Lovastatin significantly reduces serum cholesterol and LDL-serum levels. It also slows progression of coronary atherosclerosis. However, serum HDL levels are only slightly increased following lovastatin administration. The mechanism of the LDL-lowering effect may involve both reduction of VLDL concentration and induction of cellular expression of LDL-receptor, leading to reduced production and/or increased catabolism of LDL. Side effects, including liver and kidney dysfunction are associated with the use of these drugs.

Niacin, also known as nicotinic acid, is a water-soluble vitamin B-complex used as a dietary supplement and antihyperlipidemic agent. Niacin diminishes production of VLDL and is effective at lowering LDL. It is used in combination with bile-acid-binding resins. Niacin can increase HDL when administered at therapeutically effective doses; however, its usefulness is limited by serious side effects.

Fibrates are a class of lipid-lowering drugs used to treat various forms of hyperlipidemia, elevated serum triglycerides, which may also be associated with hypercholesterolemia. Fibrates appear to reduce the VLDL fraction and modestly increase HDL; however, the effects of these drugs on serum cholesterol is variable. In the United States, fibrates have been approved for use as antilipidemic drugs, but have not received approval as hypercholesterolemia agents. For example, clofibrate (ATROMID-S, Wyeth-Ayerst Laboratories) is an antilipidemic agent that acts to lower serum triglycerides by reducing the VLDL fraction. Although ATROMID-S may reduce serum cholesterol levels in certain patient subpopulations, the biochemical response to the drug is variable, and is not always possible to predict which patients will obtain favorable results. ATROMID-S has not been shown to be effective for prevention of coronary heart disease. The chemically and pharmacologically related drug, gemfibrozil (LOPID, Parke-Davis), is a lipid regulating agent which moderately decreases serum triglycerides and VLDL cholesterol. LOPID also increases HDL cholesterol, particularly the HDL₂ and HDL₃ subfractions, as well as both the AI/AII-HDL fraction. However, the lipid response to LOPID is heterogeneous, especially among different patient populations. Moreover, while prevention of coronary heart disease was observed in male patients between the ages of 40 and 55 without history or symptoms of existing coronary heart disease, it is not clear to what extent these findings can be extrapolated to other patient populations (e.g., women, older and younger males). Indeed, no efficacy was observed in patients with established coronary heart disease. Serious side-effects are associated with the use of fibrates, including toxicity; malignancy, particularly malignancy of gastrointestinal cancer; gallbladder disease; and an increased incidence in non-coronary mortality. These drugs are not indicated for the treatment of patients with high LDL or low HDL as their only lipid abnormality.

Oral estrogen replacement therapy may be considered for moderate hypercholesterolemia in post-menopausal women. However, increases in HDL may be accompanied with an increase in triglycerides. Estrogen treatment is, of course, limited to a specific patient population, postmenopausal women, and is associated with serious side effects, including induction of malignant neoplasms; gall bladder disease; thromboembolic disease; hepatic adenoma; elevated blood pressure; glucose intolerance; and hypercalcemia.

Long chain carboxylic acids, particularly long chain α,ω-dicarboxylic acids with distinctive substitution patterns, and their simple derivatives and salts, have been disclosed for treating atherosclerosis, obesity, and diabetes (See, e.g., Bisgaier et al., 1998, J. Lipid Res. 39:17-30, and references cited therein; International Patent Publication WO 98/30530; U.S. Pat. No. 4,689,344; International Patent Publication WO 99/00116; and U.S. Pat. No. 5,756,344). However, some of these compounds, for example the α,107 -dicarboxylic acids substituted at their α,α′-carbons (U.S. Pat. No. 3,773,946), while having serum triglyceride and serum cholesterol-lowering activities, have no value for treatment of obesity and hypercholesterolemia (U.S. Pat. No. 4,689,344).

U.S. Pat. No. 4,689,344 discloses β,β,β′,β′-tetrasubstituted-α,ω-alkanedioic acids that are optionally substituted at their β,β,β′,β′ positions, and alleges that they are useful for treating obesity, hyperlipidemia, and diabetes. According to this reference, both triglycerides and cholesterol are lowered significantly by compounds such as 3,3,14,14-tetramethylhexadecane-1,16-dioic acid. U.S. Pat. No. 4,689,344 further discloses that the β,β,β′,β′-tetramethyl-alkanediols of U.S. Pat. No. 3,930,024 also are not useful for treating hypercholesterolemia or obesity.

Other compounds are disclosed in U.S. Pat. No. 4,711,896. In U.S. Pat. No. 5,756,544, α,ω-dicarboxylic acid-terminated dialkane ethers are disclosed to have activity in lowering certain plasma lipids, including Lp(a), triglycerides, VLDL-cholesterol, and LDL-cholesterol, in animals, and elevating others, such as HDL-cholesterol. The compounds are also stated to increase insulin sensitivity. In U.S. Pat. No. 4,613,593, phosphates of dolichol, a polyprenol isolated from swine liver, are stated to be useful in regenerating liver tissue, and in treating hyperuricuria, hyperlipemia, diabetes, and hepatic diseases in general.

U.S. Pat. No. 4,287,200 discloses azolidinedione derivatives with anti-diabetic, hypolipidemic, and anti-hypertensive properties. However, these administration of these compounds to patients can produce side effects such as bone marrow depression, and both liver and cardiac cytotoxicity. Further, the compounds disclosed by U.S. Pat. No. 4,287,200 stimulate weight gain in obese patients.

It is clear that none of the commercially available cholesterol management drugs has a general utility in regulating lipid, lipoprotein, insulin and glucose levels in the blood. Thus, compounds that have one or more of these utilities are clearly needed. Further, there is a clear need to develop safer drugs that are efficacious at lowering serum cholesterol, increasing HDL serum levels, preventing coronary heart disease, and/or treating existing disease such as atherosclerosis, obesity, diabetes, and other diseases that are affected by lipid metabolism and/or lipid levels. There is also is a clear need to develop drugs that may be used with other lipid-altering treatment regimens in a synergistic manner. There is still a further need to provide useful therapeutic agents whose solubility and Hydrophile/Lipophile Balance (HLB) can be readily varied.

Citation or identification of any reference in Section 2 of this application is not an admission that such reference is available as prior art to the present invention.

### 3. SUMMARY OF THE INVENTION

In one embodiment, the invention relates to compounds of formula I:

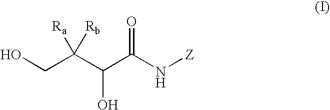

or pharmaceutically acceptable salts, solvates, clathrates, hydrates, or prodrugs thereof, wherein:

each of Rₐ and R_{b} is independently H, alkyl, alkenyl, alkynl, cycloalkyl, or aryl;

Z is (C₆-C₁₄)aryl, (C₁-C₆)alkyl, cylcoalkyl, heteroaryl, cycloheteroalkyl, or —(CH₂)ₙ—X—(CH₂)ₙ—Y;

X is O, S, Se, C(O), C(H)F, CF₂, S(O), NH, O—P(O)(OH)—O, NH—C(O)—NH or NH—C(S)—NH;

Y is —COOH, COO—{(C₁-C₆)alkyl}, COO—{(C₆-C₁₄)aryl}, —COO-(cycloalkyl), —COO-(heteroaryl). —COO-(heterocycloalkyl), —OH, —OPO₃H, —OP₂O₆H₂, —OPO₃-(nucleotide), —OP₂O₆(H)-(nucleotide), or

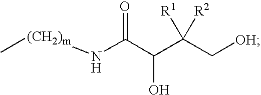

either (a) R¹ is hydrogen, methyl, or phenyl; and R² is methyl or phenyl; or (b) R¹ and R² are taken together to form a cycloalkyl ring of 3 to 6 carbons;

n and m are independently an integer from 0 to 6.

In another embodiment, the invention relates to compounds of formula II:

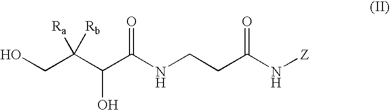

or pharmaceutically acceptable salts, solvates, clathrates, hydrates, or prodrugs thereof, wherein:

each of Rₐ and R_{b} is independently H, alkyl, alkenyl, alkynl, cycloalkyl, or aryl;

Z is (C₆-C₁₄)aryl, (C₁-C₆)alkyl, cylcoalkyl, heteroaryl, cycloheteroalkyl, or —(CH₂)ₙ—X—(CH₂)ₙ—Y;

X is O, S, Se, C(O), C(H)F, CF₂, S(O), NH, O—P(O)(OH)—O, NH—C(O)—NH or NH—C(S)—NH;

Y is —COOH, COO—{(C₁-C₆)alkyl}, COO-{(C₆-C₁₄)aryl}, —COO-(cycloalkyl), —COO-(heteroaryl). —COO-(heterocycloalkyl), —OH, —OPO₃H, —OP₂O₆H₂, —OPO₃-(nucleotide), —OP₂O₆(H)-(nucleotide), or

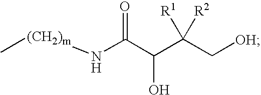

either (a) R¹ is hydrogen, methyl, or phenyl; and R² is methyl or phenyl; or (b) R¹ and R² are taken together to form a cycloalkyl ring of 3 to 6 carbons;

m is an integer from 0 to 6.

In another embodiment, the invention relates to compounds of formula III:

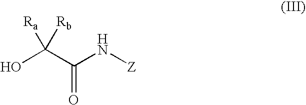

or pharmaceutically acceptable salts, solvates, clathrates, hydrates, or prodrugs thereof, wherein:

each of Rₐ and R_{b} is independently H, alkyl, alkenyl, alkynl, cycloalkyl, or aryl;

Z is (C₆-C₁₄)aryl, (C₁-C₆)alkyl, cylcoalkyl, heteroaryl, cycloheteroalkyl, or —(CH₂)ₙ—X—(CH₂)ₙ—Y;

X is O, S, Se, C(O), C(H)F, CF₂, S(O), NH, O—P(O)(OH)—O, NH—C(O)—NH or NH—C(S)—NH;

Y is —COOH, COO—{(C₁-C₆)alkyl}, COO—{(C₆-C₁₄)aryl}, —COO-(cycloalkyl), —COO-(heteroaryl). —COO-(heterocycloalkyl), —OH, —OPO₃H, —OP₂O₆H₂, —OPO₃-(nucleotide), —OP₂O₆(H)-(nucleotide), or

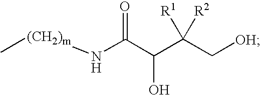

either (a) R¹ is hydrogen, methyl, or phenyl; and R² is methyl or phenyl; or (b) R¹ and R² are taken together to form a cycloalkyl ring of 3 to 6 carbons;

m is an integer from 0 to 6.

The compounds of formula I, formula II, formula III, and pharmaceutically acceptable salts, solvates, hydrates, clathrates, or prodrugs thereof are Acyl coenzyme-A mimics and/or are useful for treating or preventing cardiovascular diseases, dyslipidemias, dyslipoproteinemias, disorders of glucose metabolism, Alzheimer's Disease, Syndrome X, PPAR-associated disorders, septicemia, thrombotic disorders, obesity, pancreatitis, hypertension, renal diseases, cancer, inflammation, bacterial infection and impotence.

The compounds of formula I, formula II, formula III, and pharmaceutically acceptable salts, solvates, hydrates, clathrates, or prodrugs thereof are Acyl coenzyme-A mimics and/or are useful for increasing a patient's HDL cholesterol level, lowering a patient's LDL cholesterol level. lowering a patient's VLDL cholesterol level, lowering a patient's triglyceride level, lowering a patient's insulin level, lowering a patient's glucose level, increasing a patient's ketone body level, inhibiting fatty acid synthesis in a patient, and inhibiting cholesterol synthesis in a patient.

A further embodiment of the invention provides for pharmaceutical compositions comprising a compound of formula I, a compound of formula II, a compound of formula III, or a pharmaceutically acceptable salt, solvate, hydrate, clathrate, or prodrug thereof, and a pharmaceutically acceptable carrier.

The compositions of the invention are useful for treating or preventing cardiovascular diseases, dyslipidemias, dyslipoproteinemias, disorders of glucose metabolism, Alzheimer's Disease, Syndrome X, PPAR-associated disorders, septicemia, thrombotic disorders, obesity, pancreatitis, hypertension, renal diseases, cancer, inflammation, bacterial infection and impotence.

The compositions of the invention are useful for increasing a patient's HDL cholesterol level, lowering a patient's LDL cholesterol level. lowering a patient's VLDL cholesterol level, lowering a patient's triglyceride level, lowering a patient's insulin level, lowering a patient's glucose level, increasing a patient's ketone body level, inhibiting fatty acid synthesis in a patient, and inhibiting cholesterol synthesis in a patient.

A still further embodiment of the invention provides methods for treating or preventing a condition comprising administering to a patient in need thereof an effective amount of a compound of formula I, a compound of formula II, a compound of formula III, or a pharmaceutically acceptable salt thereof, the condition being cardiovascular diseases, dyslipidemias, dyslipoproteinemias, disorders of glucose metabolism, Alzheimer's Disease, Syndrome X, PPAR-associated disorders, septicemia, thrombotic disorders, obesity, pancreatitis, hypertension, renal diseases, cancer, inflammation, bacterial infection and impotence.

A still further embodiment of the invention provides methods for increasing a patient's HDL cholesterol level, lowering a patient's LDL cholesterol level. lowering a patient's VLDL cholesterol level, lowering a patient's triglyceride level, lowering a patient's insulin level, lowering a patient's glucose level, increasing a patient's ketone body level, inhibiting fatty acid synthesis in a patient, or inhibiting cholesterol synthesis in a patient comprising administering to a patient in need thereof an effective amount of a compound of formula I, a compound of formula II, a compound of formula III, or a pharmaceutically acceptable salt thereof.

Another embodiment of the invention encompasses a method of obtaining an acyl coenzyme A mimic, comprising determining whether a test compound binds to or inhibits the activity of a fatty acid ligase, wherein a test compound that binds to or inhibits the activity of a fatty acid ligase is an acyl coenzyme A mimic.

A further embodiment of the invention encompasses a method of obtaining an acyl coenzyme A mimic, comprising comparing binding of a test compound to a short chain fatty acid ligase versus binding of a test compound to a long chain fatty acid ligase, wherein a test compound that preferentially binds to the short chain fatty acid ligase is an acyl coenzyme A mimic.

A still further embodiment of the invention encompasses method of obtaining an acyl coenzyme A mimic, comprising comparing inhibition of a short chain fatty acid ligase by a test compound versus inhibition of the activity of a long chain fatty acid ligase by the test compound, wherein a test compound that preferentially inhibits the short chain fatty acid ligase is an acyl coenzyme A mimic.

In one embodiment, the present invention is directed toward a method for obtaining compounds that bind to and/or inhibit an enzyme that catalyzes the formation of, or the metabolism of an acyl coenzyme A molecule.

In a preferred embodiment, the present invention is directed toward a method for obtaining compounds that are inhibitors of short-chain acyl-coenzyme A ligases. This method comprises the steps of (1) docking a three-dimensional structure of a test compound with a three-dimensional structure of a substrate binding site of a short-chain acyl-coenzyme A ligase and determining a first binding energy value for this interaction; and (2) docking the three-dimensional structure of the test compound with a three-dimensional structure of a substrate binding site of a long-chain acyl-coenzyme A ligase and determining a second binding energy value for this interaction. This method may further comprise determining the ratio of the first binding energy value to the second binding energy value.

In another embodiment, the present invention is directed toward a method for obtaining acyl coenzyme A mimics that are selective inhibitors of short-chain acyl-coenzyme A ligases in which a three-dimensional structure of a test compound is docked with a three-dimensional structure of a consensus substrate binding site derived from a set of short-chain acyl-coenzyme A ligases and determining a first binding energy value for this interaction. The three-dimensional structure of the test compound is also docked with a three-dimensional structure of a consensus substrate binding site derived from a set of long-chain acyl-coenzyme A ligases and a second binding energy value is determined. This method may further comprise the step of determining the ratio of the first binding energy value to the second binding energy value.

In still another embodiment, the present invention is directed toward a method of obtaining compounds that are acyl coenzyme A mimics that are selective inhibitors of short-chain acyl-coenzyme A metabolizing enzymes. This method comprises docking a three-dimensional structure of a test compound with a three-dimensional structure of a substrate binding site of a short-chain acyl-coenzyme A metabolizing enzyme and determining a first binding energy value for this interaction. In addition, this method comprises docking the three-dimensional structure of the test compound with a three-dimensional structure of a substrate binding site of a long-chain acyl-coenzyme A metabolizing enzyme and determining a second binding energy value for this interaction. This method further comprises the step of determining the ratio of the first binding energy value to the second binding energy value. If this ratio is greater than one, the test compound is deemed to be a selective inhibitor of the short-chain acyl coenzyme A ligase tested. In preferred embodiments, the ratio, is at least 2, at least 10, and at least 100.

In a still further embodiment, the present invention is directed toward obtaining compounds that are acyl coenzyme A mimics that are selective inhibitors of short-chain acyl-coenzyme A metabolizing enzymes in which a three-dimensional structure of a test compound is docked with a three-dimensional structure of a consensus substrate binding site derived from a set of short-chain acyl-coenzyme A metabolizing enzymes and determining a first binding energy value therefor. This method further comprises the step of docking the three-dimensional structure of the test compound with a three-dimensional structure of a consensus substrate binding site derived from a set of long-chain acyl-coenzyme A metabolizing enzymes and determining a second binding energy value this interaction. The method may also comprise determining the ratio of the first binding energy value to the second binding energy value.

Accordingly, the present invention is also directed to a method of treating or preventing a condition in a patient, comprising administering to a patient in need of such treatment or prevention, a therapeutically effective amount of a compound or a pharmaceutically acceptable salt thereof identified according to the methods disclosed herein for obtaining acyl coenzyme A mimics that are selective inhibitors of short-chain acyl-coenzyme A ligases and for obtaining acyl coenzyme A mimics that are selective inhibitors of short-chain acyl-coenzyme A metabolizing enzymes. In certain aspects of this embodiment, the condition to be treated or prevented is selected from the group consisting of cardiovascular disease, dyslipidemia, dyslipoproetinemia, glucose metabolism disorder, Alzheimer's disease, Syndrome X or Metabolic Syndrome, septicemia, thrombotic disorder, peroxisome proliferator activated receptor associated disorder, obesity, hypertension, pancreatitis, renal disease, cancer, inflammation, bacterial infection, impotence, and combinations thereof. In a further aspect of this embodiment, the patient is a human.

Yet another embodiment of the invention encompasses a method of obtaining an acyl coenzyme A mimic, comprising:

a. contacting a short chain fatty acid ligase with a test compound;

b. contacting a long chain fatty acid ligase with the test compound; and

C. determining whether the test compound selectively binds to or inhibits the activity of the short chain fatty acid ligase.

In another embodiment, the compounds of the invention can be co-administered with a second or third active agent as described in U.S. Provisional Application No. 60/393,184, the entire disclosure of which is incorparated herein by reference.

The present invention can be understood more fully by reference to the detailed description and examples, which are intended to exemplify nonlimiting embodiments of the invention.

### 5. Detailed Description of the Invention

### 5.1. Definitions and Abbreviations

Apo(a): apolipoprotein(a)

Apo A-I: apolipoprotein A-I

Apo B: apolipoprotein B

Apo E: apolipoprotein E

FH: Familial hypercholesterolemia

FCH: Familial combined hyperlipidemia

GDM: Gestational diabetes mellitus

HDL: High density lipoprotein

IDL: Intermediate density lipoprotein

IDDM: Insulin dependent diabetes mellitus

LDH: Lactate dehdyrogenase

LDL: Low density lipoprotein

Lp(a): Lipoprotein (a)

MODY: Maturity onset diabetes of the young

NIDDM: Non-insulin dependent diabetes mellitus

PPAR: Peroxisome proliferator activated receptor

RXR: Retinoid X receptor

VLDL: Very low density lipoprotein

Compounds of the invention can contain one or more chiral centers and/or double bonds and, therefore, can exist as stercoisomers, such as enantiomers, diastereomers, or geometric isomers such as double-bond isomers. According to the invention, the chemical structures depicted herein, and therefore the compounds of the invention, encompass all of the corresponding compound's enantiomers and stereoisomers, that is, both the stereomerically pure form (e.g., geometrically pure, enantiomerically pure, or diastereomerically pure) and enantiomenc and stereoisomeric mixtures.

As used herein and unless otherwise indicated, the term “therapeutically effective” refers to an amount of a compound of the invention or a pharmaceutically acceptable salt, solvate, clathrate, or prodrug thereof to cause an amelioration of a disease or disorder, or at least one discernible symptom thereof. “therapeutically effective” refers to an amount of a compound of the invention or a pharmaceutically acceptable salt, solvate, clathrate, or prodrug thereof to result in an amelioration of at least one measurable physical parameter, not necessarily discernible by the patient. In yet another embodiment, the term “therapeutically effective” refes to an amount of a compound of the invention or a pharmaceutically acceptable salt, solvate, clathrate, or prodrug thereof to inhibit the progression of a disease or disorder, either physically, e.g., stabilization of a discernible symptom, physiologically, e.g., stabilization of a physical parameter, or both. In yet another embodiment, the term “therapeutically effective” refes to an amount of a compound of the invention or a pharmaceutically acceptable salt, solvate, clathrate, or prodrug thereof resulting in delaying the onset of a disease or disorder.

In certain embodiments, the compounds and compositions of the invention are administered to an animal, preferably a human, as a preventative measure against such diseases. As used herein, the term “prophylactically effective” refers to an amount of a compound of the invention or a pharmaceutically acceptable salt, solvate, clathrate, or prodrug thereof causing a reduction of the risk of acquiring a given disease or disorder. In a preferred mode of the embodiment, the compositions of the present invention are administered as a preventative measure to an animal, preferably a human, having a genetic predisposition to a cholesterol, dyslipidemia, or related disorders including, but not limited to, cardiovascular disease; artherosclerosis; stroke; peripheral vascular disease; dyslipidemia; dyslipoproteinemia; restenosis; a disorder of glucose metabolism; Alzheimer's Disease; Syndrome X; a peroxisome proliferator activated receptor-associated disorder; septicemia; a thrombotic disorder; obesity; pancreatitis; hypertension; renal disease; cancer; inflammation; inflammatory muscle diseases, such as polymylagia rheumatica, polymyositis, and fibrositis; impotence; gastrointestinal disease; irritable bowel syndrome; inflammatory bowel disease; inflammatory disorders, such as asthma, vasculitis, ulcerative colitis, Crohn's disease, Kawasaki disease, Wegener's granulomatosis, (RA), systemic lupus erythematosus (SLE), multiple sclerosis (MS), and autoimmune chronic hepatitis; impotence; arthritis, such as rheumatoid arthritis, juvenile rheumatoid arthritis, and osteoarthritis; osteoporosis, soft tissue rheumatism, such as tendonitis; bursitis; autoimmune disease, such as systemic lupus and erythematosus; scleroderma; ankylosing spondylitis; gout; pseudogout; non-insulin dependent diabetes mellitus (NIDDM); septic shock; polycystic ovarian disease; hyperlipidemias, such as familial hypercholesterolemia (FH), familial combined hyperlipidemia (FCH); lipoprotein lipase deficiencies, such as hypertriglyceridemia, hypoalphalipoproteinemia, and hypercholesterolemia; lipoprotein abnormalities associated with diabetes; lipoprotein abnormalities associated with obesity; and lipoprotein abnormalities associated with Alzheimer's Disease. Examples of such genetic predispositions include but are not limited to the ε4 allele of apolipoprotein E, which increases the likelihood of Alzheimer's Disease; a loss of function or null mutation in the lipoprotein lipase gene coding region or promoter (e.g., mutations in the coding regions resulting in the substitutions D9N and N291S; for a review of genetic mutations in the lipoprotein lipase gene that increase the risk of cardiovascular diseases, dyslipidemias and dyslipoproteinemias, see Hayden and Ma, 1992, Mol. Cell Biochem. 113:171-176); and familial combined hyperlipidemia and familial hypercholesterolemia. In another method of the invention, the compounds of the invention or compositions of the invention are administered as a preventative measure to a patient having a non-genetic predisposition to a cholesterol, dyslipidemia, or related disorders. Examples of such non-genetic predispositions include but are not limited to cardiac bypass surgery and percutaneous transluminal coronary angioplasty, which often lead to restenosis, an accelerated form of atherosclerosis; diabetes in women, which often leads to polycystic ovarian disease; and cardiovascular disease, which often leads to impotence. Accordingly, the compositions of the invention may be used for the prevention of one disease or disorder and concurrently treating another (e.g., prevention of polycystic ovarian disease while treating diabetes; prevention of impotence while treating a cardiovascular disease). Without being limited by theory it is believed that pantethine or a derivative thereof is effective when administered to a patient for more than thirty days. Accordingly, the invention encompasses methods of treating, preventing, or managing a cholesterol, dyslipidemia, or related disorder, which comprises administering for at least thirty days to a patient in need of such treatment, prevention, or management an effective amount of pantethine, or a derivative thereof, and a second active agent or a pharmaceutically acceptable salt, solvate, clathrate, polymorph, prodrug, or pharmacologically active metabolite thereof.

A compound of the invention is considered optically active or enantiomerically pure (i.e., substantially the R-form or substantially the S-form) with respect to a chiral center when the compound is about 90% ee (enantiomeric excess) or greater, preferably, equal to or greater than 95% ee with respect to a particular chiral center. A compound of the invention is considered to be in enantiomerically-enriched form when the compound has an enantiomeric excess of greater than about 80% ee with respect to a particular chiral center. A compound of the invention is considered diastereomerically pure with respect to multiple chiral centers when the compound is about 90% de (diastereomeric excess) or greater, preferably, equal to or greater than 95% de with respect to a particular chiral center. A compound of the invention is considered to be in diastereomerically-enriched form when the compound has an diastereomeric excess of greater than about 80% de with respect to a particular chiral center. As used herein, a racemic mixture means about 50% of one enantiomer and about 50% of is corresponding enantiomer relative to all chiral centers in the molecule. Thus, the invention encompasses all enantiomerically-pure, enantiomerically-enriched, diastereomerically pure, diastereomerically enriched, and racemic mixtures of compounds of Formula I and pharmaceutically acceptable salts thereof.

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of stereoisomer of the compound and less than about 20% by weight of other stereoisomers the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

As used herein and unless otherwise indicated, the term “enantiomerically pure” means a stereomerically pure composition or compound. Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

As used herein and unless otherwise indicated, the term “racemic mixture” means about 50% of one enantiomer and about 50% of is corresponding enantiomer relative to all chiral centers in the molecule. Thus, the invention encompasses all enantiomerically-pure, enantiomerically-enriched, diastereomerically pure, diastereomerically enriched, and racemic mixtures of compounds of Formulas I, II, and III and pharmaceutically acceptable salts thereof.

The compounds of the invention are defined herein by their chemical structures and/or chemical names. Where a compound is referred to by both a chemical structure and a chemical name, and the chemical structure and chemical name conflict, the chemical structure is determinative of the compound's identity.

As used herein and unless otherwise indicated, the term “second active agent” refers to a compound or mixture of compounds that are combined and/or administered with compounds of the invention. Examples of second active agents include, but are not limited to, statins, fibrates, glitazones, biguanides, dyslipidemic controlling compounds, small peptides of the invention, and pharmaceutically acceptable salts, solvates, prodrugs thereof, and combinations thereof.

As used herein and unless otherwise indicated, the term “third active agent” refers to a compound or mixture of compounds that are combined and/or administered with compounds of the invention and a second active agent. Specific third active agents reduce a disorder such as, but not limited to, hepatotoxicity, myopathy, cataracts, or rhabdomyolysis. Examples of third active agents include, but not limited to, bile acid-binding resins; niacin; hormones and pharmaceutically acceptable salts, solvates, prodrugs thereof, and combinations thereof.

When administered to a patient, e.g., to an animal for veterinary use or for improvement of livestock, or to a human for clinical use, the compounds of the invention are administered in isolated form or as the isolated form in a pharmaceutical composition. As used herein, “isolated” means that the compounds of the invention are separated from other components of either (a) a natural source, such as a plant or cell, preferably bacterial culture, or (b) a synthetic organic chemical reaction mixture. Preferably, via conventional techniques, the compounds of the invention are purified. As used herein, “purified” means that when isolated, the isolate contains at least 95%, preferably at least 98%, of a single ether compound of the invention by weight of the isolate.

As used herein and unless otherwise indicated, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds and compositions of the invention and pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

As used herein and unless otherwise indicated, the term “pharmaceutically acceptable salt(s),” includes, but is not limited to, salts of acidic or basic groups that may be present in the compounds of the invention. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds of the invention that include an amino moiety also can form pharmaceutically acceptable salts with various amino acids, in addition to the acids mentioned above. Compounds of the invention that are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

As used herein and unless otherwise indicated, the term “pharmaceutically acceptable solvate,” means a compound of the invention or a salt thereof, that further includes a stoichiometric or non-stoichiometric amount of a solvent bound by non-covalent intermolecular forces. Preferred solvents are volatile, non-toxic, and/or acceptable for administration to humans in trace amounts. The term solvate includes hydrates and means a compound of the invention or a salt thereof, that further includes a stoichiometric or non-stoichiometric amount of water bound by non-covalent intermolecular forces and includes a mono-hydrate, dihydrate, trihydrate, tetrahydrate, and the like.

As used herein and unless otherwise indicated, the term “pharmaceutically acceptable prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise NO, NO2, ONO, and ONO2 moieties. Prodrugs can typically be prepared using well known methods, such as those described in 1 Burger's Medicinal Chemistry and Drug Discovery, 172 178, 949 982 (Manfred E. Wolff ed., 5th ed. 1995), and Design of Prodrugs (H. Bundgaard ed., Elselvier, New York 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxy-methyl, pivaloyloxymethyl, and pivaloyloxyethyl esters), lactonyl esters (such as phthalidyl and thiophthalidyl esters), lower alkoxyacyloxyalkyl esters (such as methoxycarbonyloxy-methyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, a amino acid amides, alkoxyacyl amides, and alkylaminoalkyl-carbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein and unless otherwise indicated, the term “pharmaceutically acceptable hydrate” means a compound of the invention or a salt thereof, that further includes a stoichiometric or non-stoichiometric amount of water bound by non-covalent intermolecular forces.

As used herein and unless otherwise indicated, the term “pharmaceutically acceptable clathrate” means a compound of the invention or a salt thereof in the form of a crystal lattice that contains spaces (e.g., channels) that have a guest molecule (e.g., a solvent or water) trapped within.

As used herein and unless otherwise indicated, the term “altering lipid metabolism” indicates an observable (measurable) change in at least one aspect of lipid metabolism, including but not limited to total blood lipid content, blood HDL cholesterol, blood LDL cholesterol, blood VLDL cholesterol, blood triglyceride, blood Lp(a), blood apo A-I, blood apo E and blood non-esterified fatty acids.

As used herein and unless otherwise indicated, the term “altering glucose metabolism” indicates an observable (measurable) change in at least one aspect of glucose metabolism, including but not limited to total blood glucose content, blood insulin, the blood insulin to blood glucose ratio, insulin sensitivity, and oxygen consumption.

As used herein and unless otherwise indicated, the terms “alkyl group” and “(C₁-C₆)alkyl”means a saturated, monovalent unbranched or branched hydrocarbon chain. Examples of alkyl groups include, but are not limited to, (C₁-C₆)alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl group can be unsubstituted or substituted with one or two suitable substituents.

As used herein and unless otherwise indicated, the term “alkenyl group” means a monovalent unbranched or branched hydrocarbon chain having one or more double bonds therein. The double bond of an alkenyl group can be unconjugated or conjugated to another unsaturated group. Suitable alkenyl groups include, but are not limited to (C₂-C₆)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group can be unsubstituted or substituted with one or two suitable substituents.

As used herein and unless otherwise indicated, the term “alkynyl group” means monovalent unbranched or branched hydrocarbon chain having one or more triple bonds therein. The triple bond of an alkynyl group can be unconjugated or conjugated to another unsaturated group. Suitable alkynyl groups include, but are not limited to, (C₂-C₆)alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl group can be unsubstituted or substituted with one or two suitable substituents.

As used herein and unless otherwise indicated, the terms “aryl group” and “(C₆-C₁₄)aryl” mean a monocyclic or polycyclic-aromatic radical comprising carbon and hydrogen atoms. Examples of suitable aryl groups include, but are not limited to, phenyl, tolyl, anthacenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or two suitable substituents. Preferably, the aryl group is a monocyclic ring, wherein the ring comprises 6 carbon atoms, referred to herein as “(C₆)aryl”.

As used herein and unless otherwise indicated, the term “heteroaryl group” means a monocyclic- or polycyclic aromatic ring comprising carbon atoms, hydrogen atoms, and one or more heteroatoms, preferably 1 to 3 heteroatoms, independently selected from nitrogen, oxygen, and sulfur. Illustrative examples of heteroaryl groups include, but are not limited to, pyridinyl, pyridazinyl, pyrimidinyl, pyrazyl, triazinyl, pyrrolyl, pyrazolyl, imidazolyl, (1,2,3)-and (1,2,4)-triazolyl, pyrazinyl, pyrimidinyl, tetrazolyl, ftiryl, thiophenyl, isoxazolyl, thiazolyl, furyl, phenyl, isoxazolyl, and oxazolyl. A heteroaryl group can be unsubstituted or substituted with one or two suitable substituents. Preferably, a heteroaryl group is a monocyclic ring, wherein the ring comprises 2 to 5 carbon atoms and 1 to 3 heteroatoms, referred to herein as “(C₂-C₅)heteroaryl”.

As used herein and unless otherwise indicated, the term “cycloalkyl group” means a monocyclic or polycyclic saturated ring comprising carbon and hydrogen atoms and having no carbon-carbon multiple bonds. Examples of cycloalkyl groups include, but are not limited to, (C₃-C₇)cycloalkyl groups, such as cyclopropyl, cyclobutyl, cyclopentyl, cyclohexyl, and cycloheptyl, and saturated cyclic and bicyclic terpenes. A cycloalkyl group can be unsubstituted or substituted by one or two suitable substituents. Preferably, the cycloalkyl group is a monocyclic ring or bicyclic ring.

As used herein and unless otherwise indicated, the term “heterocycloalkyl group” means a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as (C₁-C₆)heterocycloalkyl.

As used herein and unless otherwise indicated, the term “heterocyclic radical” or “heterocyclic ring” means a heterocycloalkyl group or a heteroaryl group.

As used herein and unless otherwise indicated, the term “alkoxy group”means an —O-alkyl group, wherein alkyl is as defined above. An alkoxy group can be unsubstituted or substituted with one or two suitable substituents. Preferably, the alkyl chain of an alkyloxy group is from 1 to 6 carbon atoms in length, referred to herein as “(C₁-C₆)alkoxy”.

As used herein and unless otherwise indicated, the term “aryloxy group” means an —O-aryl group, wherein aryl is as defined above. An aryloxy group can be unsubstituted or substituted with one or two suitable substituents. Preferably, the aryl ring of an aryloxy group is a monocyclic ring, wherein the ring comprises 6 carbon atoms, referred to herein as “(C₆)aryloxy”.

As used herein and unless otherwise indicated, the term “benzyl” means —CH₂-phenyl.

As used herein and unless otherwise indicated, the term “phenyl” means —C₆H₅. A phenyl group can be unsubstituted or substituted with one or two suitable substituents.

As used herein and unless otherwise indicated, the term “hydrocarbyl” group means a monovalent group selected from (C₁-C₈)alkyl, (C₂-C₈)alkenyl, and (C₂-C₈)alkynyl, optionally substituted with one or two suitable substituents. Preferably, the hydrocarbon chain of a hydrocarbyl group is from 1 to 6 carbon atoms in length, referred to herein as “(C₁-C₆)hydrocarbyl”.

As used herein and unless otherwise indicated, the term “carbonyl” group is a divalent group of the formula —C(O)—.

As used herein and unless otherwise indicated, the term “alkoxycarbonyl” group means a monovalent group of the formula —C(O)-alkoxy. Preferably, the hydrocarbon chain of an alkoxycarbonyl group is from 1 to 8 carbon atoms in length, referred to herein as a “lower alkoxycarbonyl” group.

As used herein and unless otherwise indicated, the term “carbamoyl” group means the radical —C(O)N(R′)₂, wherein R′ is chosen from the group consisting of hydrogen, alkyl, and aryl.

As used herein and unless otherwise indicated, the term “halogen” means fluorine, chlorine, bromine, or iodine. Correspondingly, the meaning of the terms “halo” and “Hal”encompass fluoro, chloro, bromo, and iodo.

As used herein and unless otherwise indicated, the term “suitable substituent” means a group that does not nullify the synthetic or pharmaceutical utility of the compounds of the invention or the intermediates usefuil for preparing them. Examples of suitable substituents include, but are not limited to: (C₁-C₈)alkyl; (C₁-C₈)alkenyl; (C₁-C₈)alkynyl; (C₆)aryl; (C₂-C₅)heteroaryl; (C₃-C₇)cycloalkyl; (C₁-C₈)alkoxy; (C₆)aryloxy; —CN; —OH; oxo; halo, —CO₂H; —NH₂; —NH((C₁-C₈)alkyl); —N((C₁-C₈)alkyl)₂; —NH((C₆)aryl); —N((C₆)aryl)₂; —CHO; —CO((C₁-C₈)alkyl); —CO((C₆)aryl); —CO₂((C₁-C₈)alkyl); and —CO₂((C₆)aryl). One of skill in the art can readily choose a suitable substituent based on the stability and pharmacological and synthetic activity of the compound of the invention.

As used herein and unless otherwise indicated, the term “nucleotide” means a group having aribose or deoxyribose sugar joined to a purine or pyrimidine base and to one or more phosphate groups. Examples of nucleotides include, but are not limited to, adenine, guanine, cytosine, thymine, uracil and thio and thiotriphosphate analogs thereof.

As used herein and unless otherwise indicated, the term “short chain acyl coenzyme A” ligase refers to an enzyme catalyzing the condensation of a C₂-C₈ carboxylic acid and coenzyme A to form a short chain acyl-coenzyme A product. Similarly, the phrase “medium chain acyl coenzyme A” ligase refers to an enzyme catalyzing the condensation of a C₁₀-C₁₆ carboxylic acid and coenzyme A to form a short chain acyl-coenzyme A product. Accordingly, the phrase “long chain acyl coenzyme A” ligase refers to an enzyme catalyzing the condensation of a carboxylic acid comprising a carbon chain of more than 16 carbon atoms and coenzyme A to form a long chain acyl-coenzyme A product.

As used herein and unless otherwise indicated, the phrases “long chain acyl coenzyme A” metabolizing enzyme, “medium chain acyl coenzyme A” metabolizing enzyme, and “long chain acyl coenzyme A” metabolizing enzyme refer to enzymes using a short-chain, medium-chain, long-chain acyl coenzyme A molecule as a substrate, respectively.

As used herein and unless otherwise indicated, the term “docking” refers to a computer-assisted method for determining and evaluating energetically-favorable interactions between a biological macromolecule and a ligand the interacts with that biological macromolecule. As used herein, the term ligand encompasses both natural substrates as well as non-substrate inhibitors of the biochemical activity of the biological macromolecule to which it binds.

### 5.2. Compounds of Formula I

In another embodiment, the invention relates to compounds of formula I:

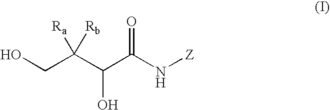

or pharmaceutically acceptable salts, solvates, clathrates, hydrates, or prodrugs thereof, wherein:

each of Rₐ and R_{b} is independently H, alkyl, alkenyl, alkynl, or aryl;

Z is (C₆-C₁₄)aryl, (C₁-C₆)alkyl, cylcoalkyl, heteroaryl, cycloheteroalkyl, or —(CH₂)ₙ—X—(CH₂)ₙ—Y;

X is O, S, Se, C(O), C(H)F, CF₂, S(O), NH, O—P(O)(OH)—O, NH—C(O)—NH or NH—C(S)—NH;

Y is —COOH, COO—{(C₁-C₆)alkyl}, COO—{(C₆-C₁₄)aryl}, —COO-(cycloalkyl), —COO-(heteroaryl). —COO-(heterocycloalkyl), —OH, —OPO₃H, —OP₂O₆H₂, —OPO₃-(nucleotide), —OP₂O₆(H)-(nucleotide), or

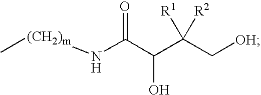

either (a) R¹ is hydrogen, methyl, or phenyl; and R² is methyl or phenyl; or (b) R¹ and R² are taken together to form a cycloalkyl ring of 3 to 6 carbons;

n and m are independently an integer from 0 to 6.

### 5.4. Compounds of Formula II and III

In another embodiment, the invention relates to compounds of formula II:

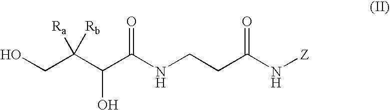

or pharmaceutically acceptable salts, solvates, clathrates, hydrates, or prodrugs thereof, wherein:

each of Rₐ and R_{b} is independently H, alkyl, alkenyl, alkynl, or aryl;

Z is (C₆-C₁₄)aryl, (C₁-C₆)alkyl, cylcoalkyl, heteroaryl, cycloheteroalkyl, or —(CH₂)ₙ—X—(CH₂)ₙ—Y;

X is O, S, Se, C(O), C(H)F, CF₂, S(O), NH, O—P(O)(OH)—O, NH—C(O)—NH or NH—C(S)—NH;

Y is —COOH, COO—{(C₁-C₆)alkyl}, COO—{(C₆-C₁₄)aryl}, —COO-(cycloalkyl), —COO-(heteroaryl). —COO-(heterocycloalkyl), —OH, —OPO₃H, —OP₂O₆H₂, —OPO₃-(nucleotide), —OP₂O₆(H)-(nucleotide), or

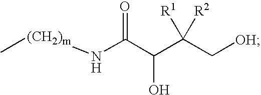

either (a) R¹ is hydrogen, methyl, or phenyl; and R² is methyl or phenyl; or (b) R¹ and R² are taken together to form a cycloalkyl ring of 3 to 6 carbons;

m is an integer from 0 to 6.

In another embodiment, the invention relates to compounds of formula III:

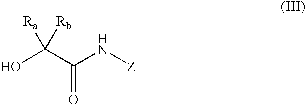

or pharmaceutically acceptable salts, solvates, clathrates, hydrates, or prodrugs thereof, wherein:

each of Rₐ and R_{b} is independently H, alkyl, alkenyl, alkynl, or aryl;

Z is (C₆-C₁₄)aryl, (C₁-C₆)alkyl, cylcoalkyl, heteroaryl, cycloheteroalkyl, or —(CH₂)ₙ—X—(CH₂)ₙ—Y;

X is O, S, Se, C(O), C(H)F, CF₂, S(O), NH, O—P(O)(OH)—O, NH—C(O)—NH or NH—C(S)—NH;

Y is —COOH, COO—{(C₁-C₆)alkyl}, COO—{(C₆-C₁₄)aryl}, —COO-(cycloalkyl), —COO-(heteroaryl). —COO-(heterocycloalkyl), —OH, —OPO₃H, —OP₂O₆H₂, —OPO₃-(nucleotide), —OP₂O₆(H)-(nucleotide), or

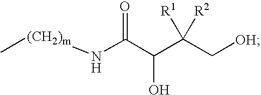

either (a) R¹ is hydrogen, methyl, or phenyl; and R² is methyl or phenyl; or (b) R¹ and R² are taken together to form a cycloalkyl ring of 3 to 6 carbons;

m is an integer from 0 to 6.

### 5.5. Illustrative Compounds of Formulas I-III

Illustrative compounds of formulas I-III include, but are not limited to:

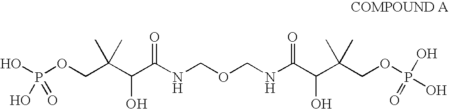

### Phosphoric acid mono-(3-hydroxy-3-{[(2-hydroxy-3,3-dimethyl-4-phosphonooxy-butyrylamino)-methoxymethyl]-carbamoyl}-2,2-dimethyl-propyl) ester

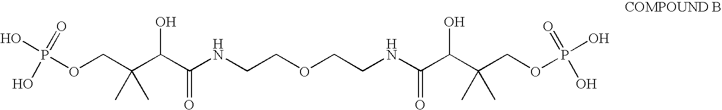

### Phosphoric acid mono-(3-hydroxy-3-{2-[2-(2-hydroxy-3,3-dimethyl-4-phosphonooxy-butyrylamino)-ethoxy]-ethylcarbamoyl}-2,2-dimethyl-propyl) ester

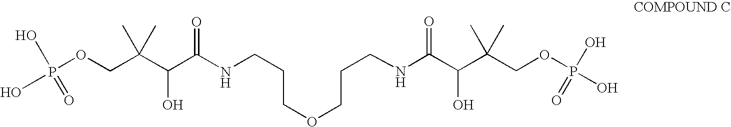

### Phosphoric acid mono-(3-hydroxy-3-{3-[3-(2-hydroxy-3,3-dimethyl-4-phosphonooxy-butyrylamino)-propoxy]-propylcarbamoyl}-2,2-dimethyl-propyl) ester

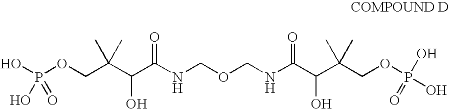

### Phosphoric acid mono-{3-hydroxy-3-[3-(2-hydroxy-3,3-dimethyl-4-phosphonooxy-b utyrylamino)-2-oxo-propylcarbamoyl]-2,2-dimethyl-propyl} ester

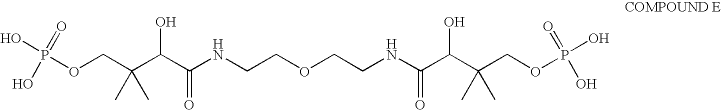

### Phosphoric acid mono-{3-hydroxy 3-[5-(2-hydroxy-3,3 dimethyl-4-phosphonooxy-butyrylamino)-3-oxo-pentylcarbamoyl]-2,2-dimethyl-propyl} ester

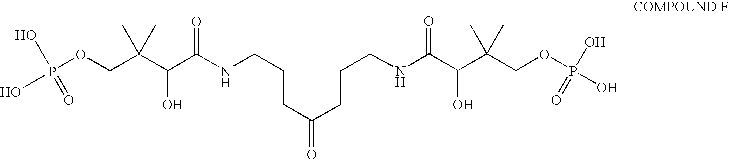

### Phosphoric acid mono-{3-hydroxy-3-[7-(2-hydroxy-3,3-dimethyl-4-phosphonooxy-butyrylamino)-4-oxo-heptylcarbamoyl]-2,2-dimethyl-propyl} ester

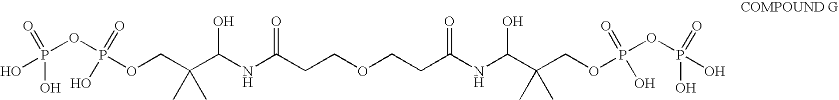

### Diphosphoric acid mono-(3-hydroxy-3-{3-[2-(1-hydroxy-2,2-dimethyl-3-phosphonooxy-propylcarbamoyl)-ethoxy]-propionylamino}-2,2-dimethyl-propyl) ester

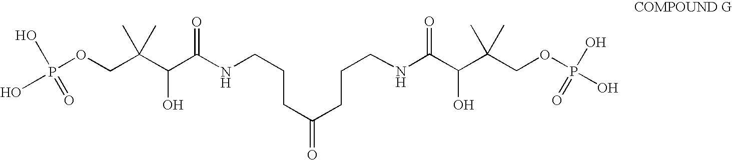

### Diphosphoric acid mono-(3-hydroxy-3-{3-[3-(2-hydroxy-3,3-dimethyl-4-phosphonooxy-butyrylamino)-propoxy]-propylcarbamoyl}-2,2-dimethyl-propyl) ester

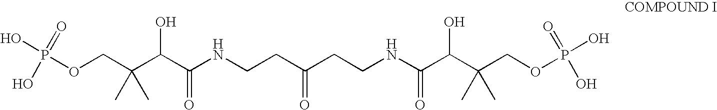

### Phosphoric acid mono-{3-hydroxy-3-[5-(2-hydroxy-3,3-dimethyl-4-phosphonooxy-butyrylamino)-3-oxo-pentylcarbamoyl]-2,2-dimethyl-propyl} ester

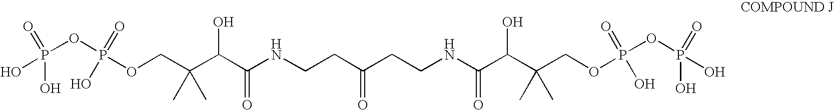

### Diphosphoric acid mono-{3-hydroxy-3-[5-(2-hydroxy-3,3-dimethyl-4-phosphonooxy-butyrylamino)-3-oxo-heptylcarbamoyl]-2,2-dimethyl-propyl} ester

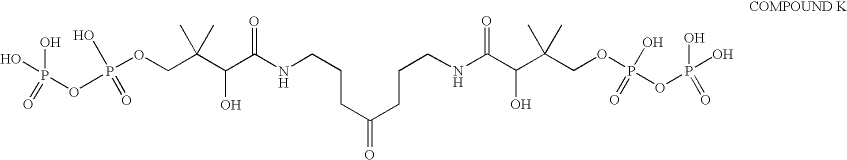

### Diphosphoric acid mono-{3-hydroxy-3-[7-(2-hydroxy-3,3-dimethyl-4-phosphonooxy-butyrylamino)-4-oxo-heptylcarbamoyl]-2,2-dimethyl-propyl} ester

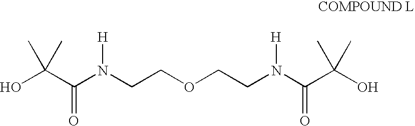

### bis(3-Aza-4-oxo-5-hydroxy-5-methylhexyl)ether

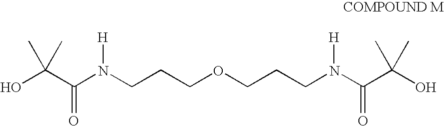

### bis(4-Aza-5-oxo-6-hydroxy-6-methylheptyl)ether

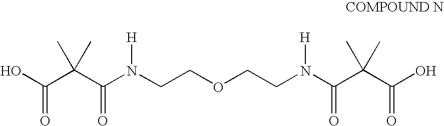

### bis(3-Aza-4-oxo-5-hydroxy-5-methylhexyl)ether

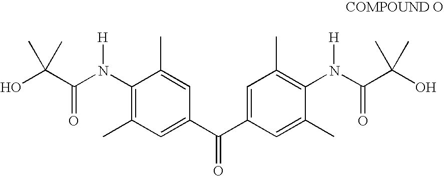

### bis[4-(2-Hydroxy-2-methylpropanamido)-3,5-dimethylphenyl]ketone

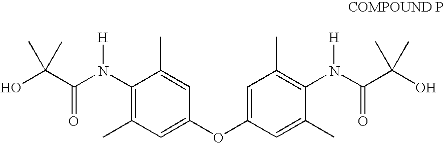

### bis[N-(2-hydroxy-2-methylpropanoyl)-3,5-dimethyl-4-anilino]ether

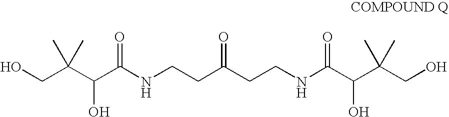

### N,N ′-(2,4-Dihydroxy-3,3-dimethylbutanoyl)3-oxo-pentan-1,5-diamine

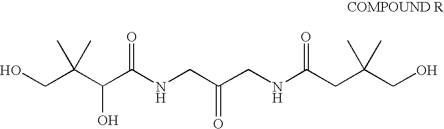

### ((2-Aza-3-oxo-4,6-dihydroxy-5,5-dimethyl)ketone

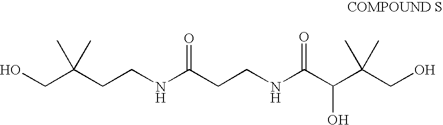

### 2,2,12,12-Tetramethyl-4,8-dioxo-5,9-diazatridecan-1,2,13-triol

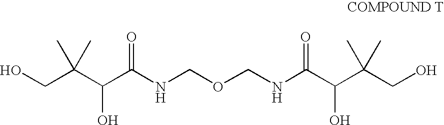

### (2-Aza-3-oxo-4,6-dihydroxy)ether

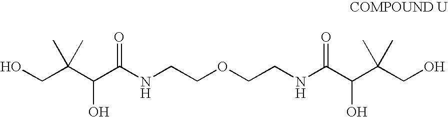

### bis(3-Aza-4-oxo-5,7-dihyroxyheptyl)ether

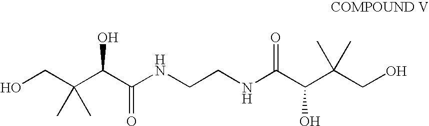

### (R,S)-N-[2-(2,4-Dihydroxy-3,3-dimethylbutrylamino)-ethyl]-2,4-dihydroxy-3,3-dimethylbutyramide

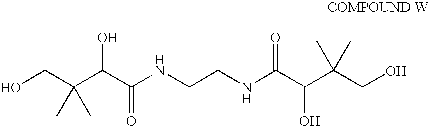

### 5,8-Diaza-4,9-dioxo-2,2,11,11-tetramethyl-dodecane-1,3,10,1 2-tetraol

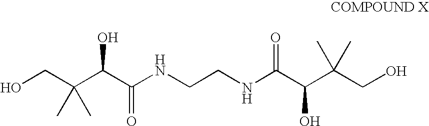

### 3R, 10R)-5,8-Diaza-4,9-dioxo-2,2,11,11-tetramethyl-1,3,10,12-tetrahydroxydodecane

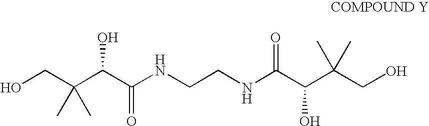

### (3S, 10S)-5,8-Diaza-4,9-dioxo-2,2,11,11-tetramethyl-1,3,10,12-tetrahydroxydodecane

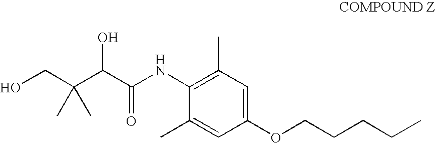

### N-(2,6-dimethyl-4-pentyloxy-phenyl)-2,4-dihydroxy-3,3-dimethyl-butyramide

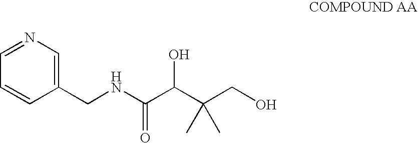

### 2,4-dihydroxy-3,3-dimethyl-N-pyridin-3ylmethyl-butyramide

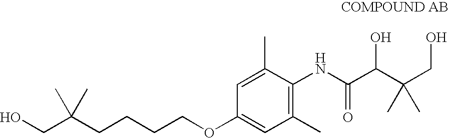

### 2,4-Dihydroxy-N-[4-(6-hydroxy-5,5-dimethyl-hexyloxy)-2,6-dimethyl-phenyl]-3,3-dimethyl-butyramide

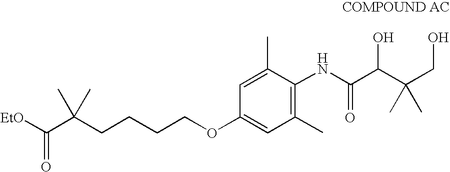

### 6-[4-(2,4-dihydroxy-3,3-dimethyl-butyrylamino)-3,5-dimethyl-phenoxy]-2,2-dimethyl-hexanoic acid ethyl ester

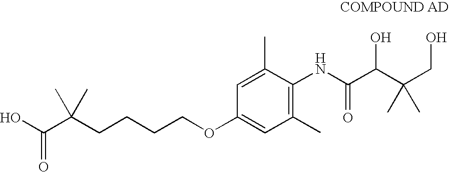

### 6-4-[(2,4-dihydroxy-3,3-dimethylbutanoyl)amino]-3,5-dimethylphenoxy-2,2-dimethylhexanoic acid

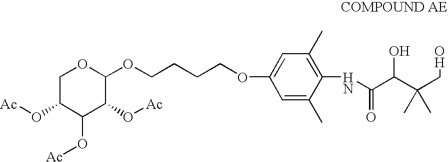

### 2,4-dihydroxy-N-{4-[4-(2,3,4-tri-O-acetyl-a-D-xylopyransoyl)-butoxy]-2,6-dimethyl-phenyl}-3,3-dimethyl-butyramide

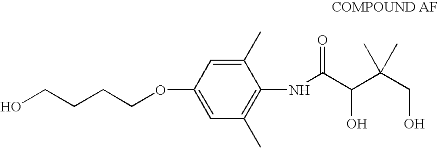

### 2,4-Dihydroxy-N-[4-(4-hydroxy-butoxy)-2,6-dimethyl-phenyl]-3,3-dimethyl-butyramide

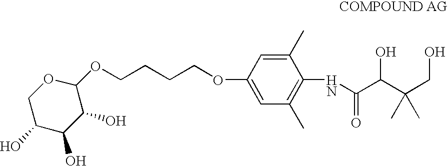

### N-{2,6-Dimethyl-4-[4-(3,4,5-trihydroxy-tetrahydro-pyran-2-yloxy)-butoxy]-phenyl}-2,4-dihydroxy-3,3-dimethyl-butyramide

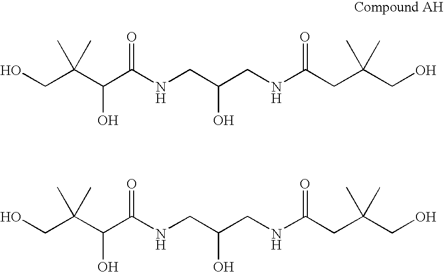

### 2,4-Dihydroxy-N-[2-hydroxy-3-(4-hydroxy-3,3-dimethyl-butyrylamino)-propyl]-3,3-dimethyl-butyramide

### 5.6. Synthesis of the Compounds of Formulas I-III

The compounds of the invention can be obtained via the synthetic methodology illustrated in Schemes 1-11. Starting materials useful for preparing the compounds of the invention and intermediates therefor are commercially available or can be prepared by well known synthetic methods.

Scheme 1 illustrates the preparation of α,γ-hydroxyamide derivatives of type I. The most common method used is the reaction of a primary amine with pantolactone in conditions similar to the ones described in Fizet, C. Helv. Chim. Acta 1986, 69, 404. Racemic mixtures and stercoisomers are obtained by this route.

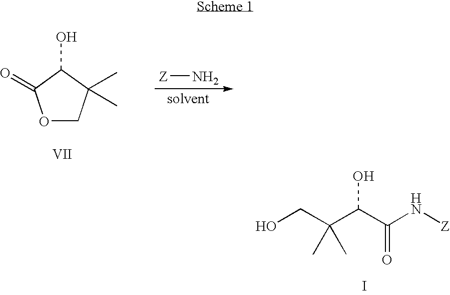

Scheme 2 presents the synthesis of symmetrical bis-α,γ-hydroxyamide derivatives derivatives of type I obtained either by concerted disubstitution-ring opening at both sites (when racemic, R-R and S-S), or by stepwise substitution for the preparation of the meso form (vide infra).

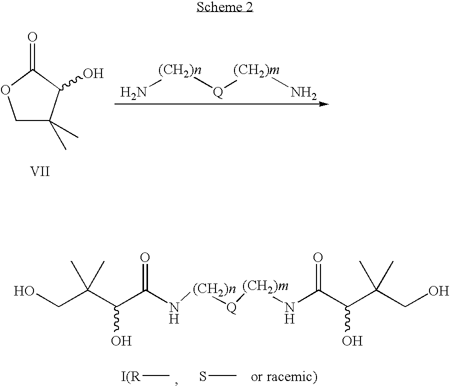

Compounds of type I (both mono and bis-α,γ-hydroxyamide derivatives) are also obtained as described in Scheme 3, by the following reaction sequence: racemic, R- or S-pantolactone ring open in basic conditions (using as a solvent methanol, ethanol, and as a base sodium or sodium hydroxide, potassium hydroxide, preferably potassium hydroxide in methanol, at room temperature or under slight heating, preferably at room temperature) to produce salt VIII which after protection (such as t-butyldimethylsilyl chloride in diisopropylethylamine in the presence of catalytic amounts of 4-dimethylaminopyridine) and hydrolysis (same conditions as for producing VIII) gives acid X. A similar procedure is described in Morton, D. R. et al. J Org. Chem. 1978, 39, 2102. Acid X is transformed in the active species XI by treatment with N-hydroxysuccinimide and dicyclohexylcarbodiimide as described in Bergeron, R. J. et al. Tetrahedron: Assymetry 1999, 10, 4285, to activate the nucleophilic substitution with amines of type XII, which is preferably performed in anhydrous tetrahydrofuran at room temperature or under heating up to reflux. Amines of type XII are commercially available (e.g., Aldrich Chemical Co., Milwaukee, Wis.) or are obtained by methods known in the literature. Derivative XIII thus obtained is deprotected to give the desired compound of type I (only the monoderivative displayed in Scheme 3).

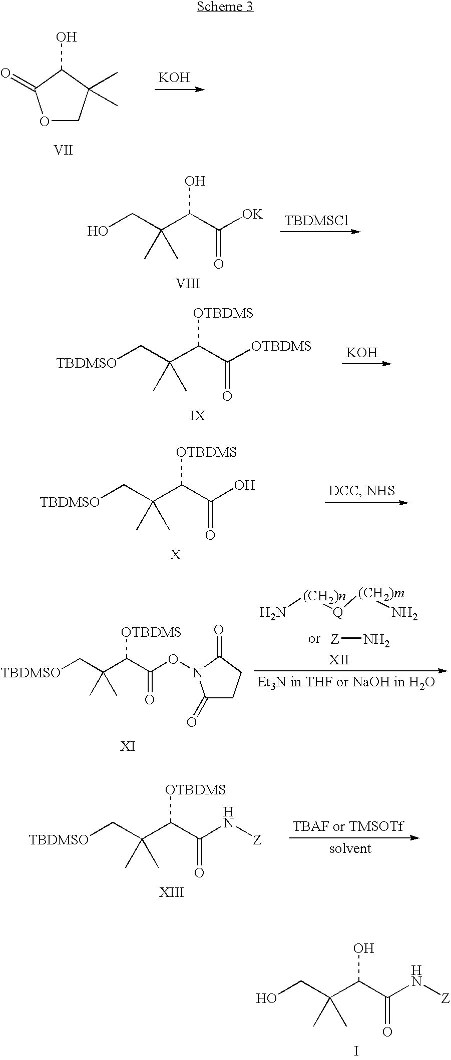

Scheme 4 illustrates the synthesis of amines XII from aldehydes XIV via the imine XV (see Wang et al. J. Org. Chem. 1995, 60, 7364, Tanaka et al. J. Med. Chem. 1998, 41, 2390, Smith and March, Advanced Organic Chemistry: Reactions, Mechanisms and Structures, 5th Ed.; Wiley: New York, 2001; p 1203, and references cited herein, and methods referenced in Larock, Comprehensive Organic Transformations, 2nd Ed., Wiley: New York 1999, p. 835). In a typical procedure, a mixture of aldehyde and ammonium formate or ammonium oxalate is heated at temperatures higher than 120° C., preferably at 140° C., until no more water is distilled off. Then the temperature of the reaction mixture is raised to over 150° C., preferably 180-200° C., for 2 to 10 hours. The reaction mixture is cooled at room temperature, treated with concentrated HCl at room temperature or higher for 2 to 6 hours, and the organic impurities extracted with an organic solvent such as diethyl-ether, t-butyl methyl ether, benzene, toluene, hexane, preferably toluene. Afterwards, the aqueous layer is made alkaline with an aqueous sodium hydroxide solution and the amine is extracted in an organic solvent and purified by methods commonly used in the field. Amines XII are also prepared from a halide XVI (X=Hal) and dibenzylamine. In a typical procedure, halide XVI is treated with dibenzylamine neat at temperatures in the range of 100 to 150° C., preferably 130° C., or in diglyme in the presence of potassium carbonate at temperatures in the range of 120 to 180° C., preferably at 140° C., until no more change in the starting material is observed by an analytical method such as but not limited to High Pressure Liquid Chromatography or Thin Layer Chromatography. When the reaction is complete, the amine is converted into a hydrochloride and is precipitated as a hydrochloride in a dry solvent such as 2-propanol. The dibenzylamine derivative XVII is treated with 10% Pd/C and ammonium formate in methanol at reflux for 2 to 24 hours, then filtration through Celite; evaporation of the solvent yields the crude amine XII, which is purified by usual methods (Purchase et al. J. Org. Chem. 1991, 56, 457-459).

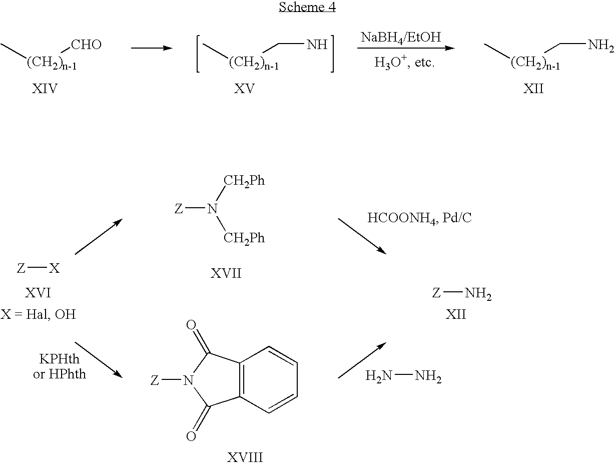

Scheme 4 also illustrates the preparation of amines of formula XII by Gabriel synthesis starting from halo-derivatives XVI (for general references see Gibson et al. Angew. Chem. 1968, 80, 986, Macholan, L. Coll. Czech. Chem. Comm. 1974, 39, 653-661 Smith and March, Advanced Organic Chemistry: Reactions, Mechanisms and Structures, 5th Ed.; Wiley: New York, 2001; p 513, and references cited herein). For an improved Gabriel synthesis, see also Sheehan et al. J. Amer. Chem. Soc. 1950, 72, 2786-2788. In a typical procedure, bromide XVI (X=Br) and potassium phthalimide in DMF are kept at room temperature or heated to 90° C. for 0.5 to 4 hours, extracted in a solvent, or precipitated by addition of water and recrystallized. The phthalimide of formula XVIII thus obtained is treated in methanol with an 85% aqueous solution of hydrazine hydrate for 15 min to one hour. Addition of water and removal of the methanol is followed by addition of HCl and heating under reflux for 1 hour, removal of crystalline phthalhydrazide by cooling to 0° C., then workup of the amine XII from the filtrate. In an alternative procedure potassium phthalimide and potassium carbonate in the presence of catalytic amounts of benzyltriethylammonium chloride in acetone are refluxed for 40 min, then bromide of formula XVI is added dropwise for 4 hr at reflux. When the reaction is complete, the mixture is subjected to separation and purification by known methods, such as chromatography or recrystallization. As a reference see Sasse et al. J. Med. Chem. 2001, 44, 694-702 and Khan J. Org. Chem. 1996, 61, 8063-8068. The reactions described above are all monitored by an analytical method such as HPLC., tlc or NMR. N-Alkylphthalimides of formula XVIII are also prepared starting from an alcohol and phthalimide in Mitsunobu conditions (Mitsunobu et al. J. Amer. Chem. Soc. 1972, 94, 679-680). In a typical procedure, an alcohol of formula XVI (X=OH) is treated with phthalimide in the presence of triphenylphosphine and diethyl azodicarboxylate in dry THF at 0° C., then the mixture is stirred overnight at room temperature. After evaporation of the solvent, the phthalimide is separated and purified in the usual manner. Subsequently, the phthalimide in ethanol is treated with hydrazine hydrate at reflux for 15 min, and then the suspension cooled, acidified and filtered. The amine of formula XII is recovered from the filtrate as a hydrochloride or as a free base by usual separation methods.

An alternative to the procedure described above is the preparation of derivatives of type I (both mono and bis-α,γ-hydroxyamide derivatives) by activation using 1-chloro-3,5-dimethoxy-s-triazine, as illustrated in Scheme 5 for bis-derivatives. Activation of XXI with 2-chloro-4,6-dimethoxy-1,3,5-triazine in the presence of N-methyl-morpholine as base in dry acetonitrile [as described in Hipskind, P.A. et. al. J. Org. Chem. 1995, 60, 7033-7036; Kaminski, Z. J. Int. J. Peptide Protein Res. 1994, 43, 312-319] cleanly leads to the triazine intermediate XIX. Treatment in situ of the intermediate with an amine at room temperature or under slight heating, preferably at room temperature, gives products of type I. In a typical procedure to a stirred solution of XXI (10 mmol) in acetonitrile (50 mL) is added, under a nitrogen atmosphere, N-methyl-morpholine (excess, 20 to 24 mmol) and 2-chloro-4,6-dimethoxy-1,3,5-triazine (excess, 20 to 24 mmol) at room temperature. After 10 to 20 hours, the amine XII (30 to 55 mmol if monoamine and 15 to 30 mmol if bis-amine) is added and the reaction mixture is stirred for 15 to 30 hours at room temperature. The reaction mixture is diluted with ethyl acetate and extracted with ice-cold 1 N HCl, then the organic layer is purified by common methods and after deprotection the product is isolated by flash chromatography, crystallization or distillation.

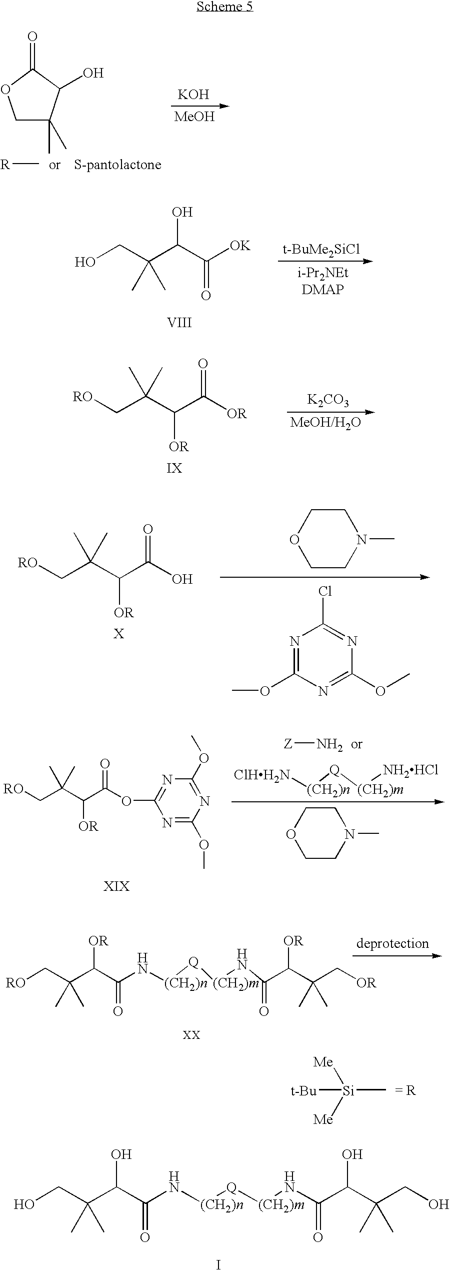

A stepwise addition of the α,γ-hydroxyamide moieties is the alternative to the above procedure illustrated by Scheme 6. The method is useful for the synthesis of chiral derivatives of type II. In a typical procedure chiral pantolactone is treated with monoprotected diamine XXIV to give the monosubstituted derivative of type XXV, as described in Fizet, C. Helv. Chim. Acta 1986, 69, 404. The intermediate after purification by a common method such as distillation, chromatography or recrystallization, is deprotected and subsequently treated with a second mol of chiral pantolactone, to give the compound of type I.

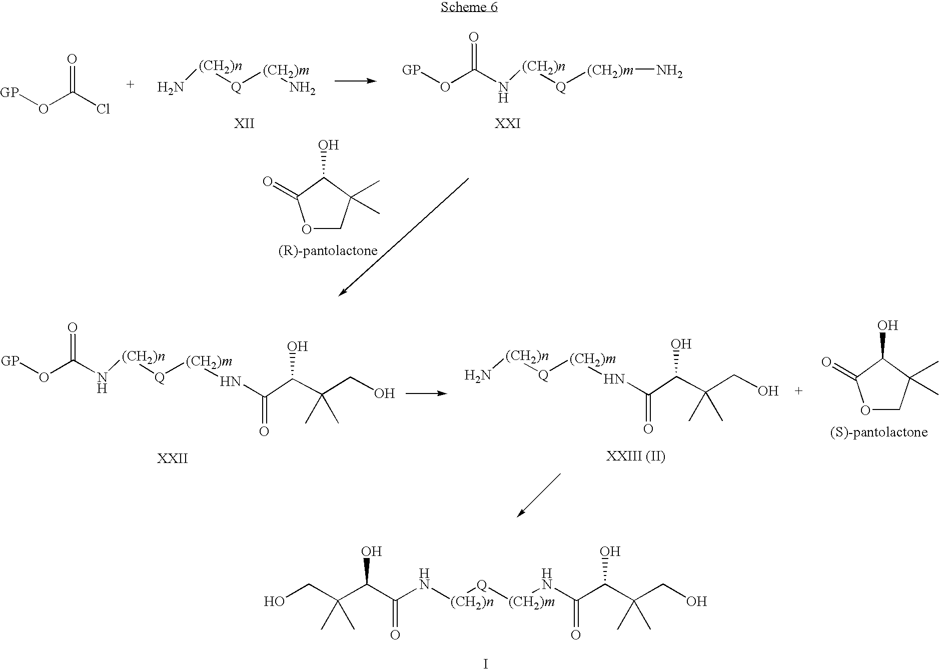

Syntheses of derivatives of type II are illustrated by Scheme 7. Sodium pantothenate is reacted with equimolar amounts of N-hydroxysuccinimide in the presence of dicyclohexylcarbodiimide in various solvents such as dimethylformamide, chloroform, dimethylsulfoxide, dichloromethane or mixtures of solvents, preferably dimethylformamide:dichloromethane (3:2), at room temperature to produce the activated ester XXVIII, which is reacted in situ with the amine XII. Similar reaction conditions are described in Haque, T. S. J. Amer. Chem. Soc. 1996, 118, 6975.

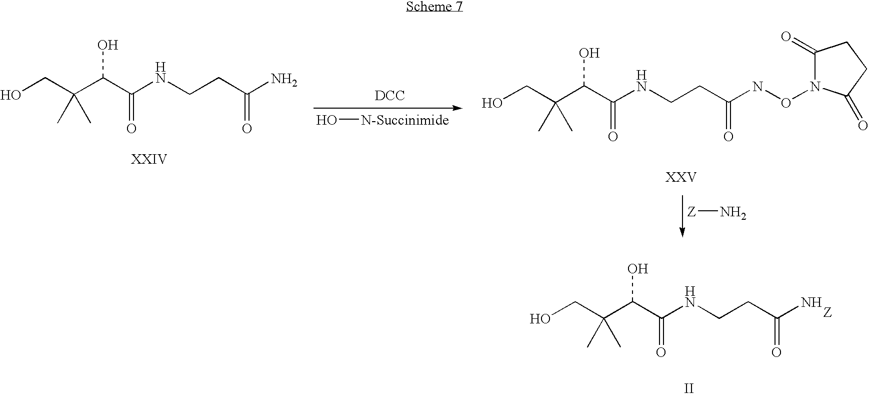

Syntheses of derivatives of type III are performed by methods similar by those described earlier in the literature for the synthesis of amides of α-hydroxyacids, e.g. Barany, G.; Merrifield, R. B. in Peptides, Gross, E.; Meienhofer, J. (Eds.), Academic Press: New York 1980, Vol. 2, p 208-211; Kleinberg, J. et al. Organic Syntheses, Collective Volume 3, Wiley, pp 516-518. Examples are found in Ratchford, A.; Lengel, C.; Fisher, I. J Amer. Chem. Soc. 1949, 71, 649; Rekker, et al. Recl. Trav. Chim. Pays-Bas 1951, 70, 14; Mulliez, M. et al. Bull. Soc. Chim. Fr. 1986, 101.

A typical synthesis of a compound of type III is described in Scheme 8. The amine XII is treated with a-hydroxybutiric ester XXIX in equimolar amounts or excess, using various solvents such as ethanol, isopropanol, THF, DMF, with or without sulfuric acid, at temperatures ranging between 50 to 200° C. Preferably α-hydroxybutiric ester is used as both reactant and solvent and the desired compounds are obtained by heating the reaction mixture at reflux. Some similar examples are given in Ratchford, A. et al. J Amer. Chem. Soc. 1949, 71, 649; Rekker, et al. Recl. Trav. Chim. Pays-Bas 1951, 70, 14; Mulliez, et al. Bull. Soc. Chim. Fr. 1986, 101.

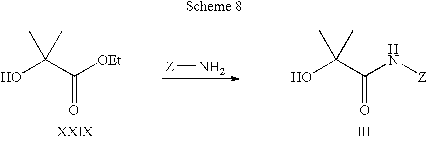

Compounds of type III are also prepared as described in Scheme 9 via 3,5,5-trimethyl-oxazolidine-2,4-dione XXX, similar to the procedure applied by Rekker, et al. Recl. Trav. Chim. Pays-Bas 1951, 70, 241-247; Davies, H. J. Chem. Soc. 1950, 30-34; and Spielman, J. Amer. Chem. Soc. 1944, 66, 1244.

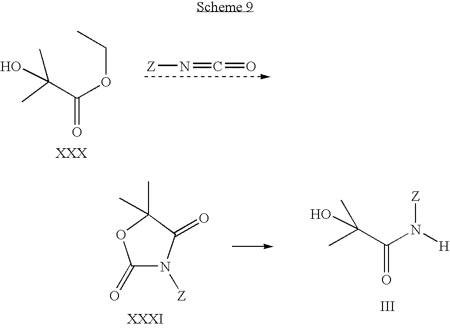

Compounds of type III are obtained from a-bromo-isobutyric acid amides obtained as illustrated in Scheme 10, in the presence of Ag₂O and H₂O by stirring the reagents in acetonitrile at room temperature for 3 to 48 hours, as described in Cavicchioni, G., Synth.Commun. 1994, 24, 2223-2228.

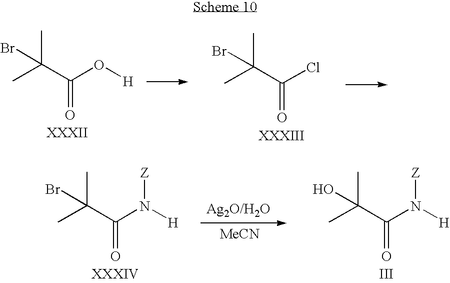

Compounds of type III are also obtained from N-alkyl-C-(trichlorotitanio)-formimidoyl chloride XXXV and propan-2-one in dichloromethane, at temperatures of —60 to 0 ° C. and in the presence of 2 N HCl (see Schiess, M. et al. Helv. Chim.Acta 1983, 66, 1618-1623) (Scheme 11).

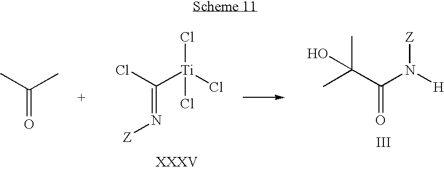

### 5.7. Therapeutic Uses of Compounds of the Invention

In accordance with the invention, the compounds of formula I, formula II, formula III or a pharmaceutically acceptable salt thereof or an acyl coenzyme-A mimic identified by a method disclosed herein (collectively, “the compounds of the invention”) are useful for administration to a patient, preferably a human, with or at risk of cardiovascular disease, a dyslipidemia, a dyslipoproteinemia, a disorder of glucose metabolism, Alzheimer's Disease, Syndrome X, a PPAR-associated disorder, septicemia, a thrombotic disorder, obesity, pancreatitis, hypertension, a renal disease, cancer, inflammation, bacterial infection or impotence. In one embodiment, “treatment” or “treating” refers to an amelioration of a disease or disorder, or at least one discernible symptom thereof. In another embodiment, “treatment” or “treating” refers to delaying the onset of a disease or disorder or inhibiting the progression thereof, either physically, e.g., stabilization of a discernible symptom, physiologically, e.g., stabilization of a physical parameter, or both.

In certain embodiments, the compounds of the invention or the compositions of the invention are administered to a patient, preferably a human, as a preventative measure against such diseases. As used herein, “prevention” or “preventing” refers to a reduction of the risk of acquiring a given disease or disorder. In a preferred mode of the embodiment, the compositions of the present invention are administered as a preventative measure to a patient, preferably a human having a genetic predisposition to a cardiovascular disease, a dyslipidemia, a dyslipoproteinemia, a disorder of glucose metabolism, Alzheimer's Disease, Syndrome X, a PPAR-associated disorder, septicemia, a thrombotic disorder, obesity, pancreatitis, hypertension, a renal disease, cancer, inflammation, bacterial infection or impotence. Examples of such genetic predispositions include but are not limited to the ε⁴ allele of apolipoprotein E, which increases the likelihood of Alzheimer's Disease; a loss of function or null mutation in the lipoprotein lipase gene coding region or promoter (e.g., mutations in the coding regions resulting in the substitutions D9N and N291S; for a review of genetic mutations in the lipoprotein lipase gene that increase the risk of cardiovascular diseases, dyslipidemias and dyslipoproteinemias, see Hayden and Ma, 1992, Mol. Cell Biochem. 113:171-176); and familial combined hyperlipidemia and familial hypercholesterolemia.

In another preferred mode of the embodiment, the compounds of the invention or compositions of the invention are administered as a preventative measure to a patient having a non-genetic predisposition to a cardiovascular disease, a dyslipidemia, a dyslipoproteinemia, a disorder of glucose metabolism, Alzheimer's Disease, Syndrome X, a PPAR-associated disorder, septicemia, a thrombotic disorder, obesity, pancreatitis, hypertension, a renal disease, cancer, inflammation, bacterial infection or impotence. Examples of such non-genetic predispositions include but are not limited to cardiac bypass surgery and percutaneous transluminal coronary angioplasty, which often lead to restenosis, an accelerated form of atherosclerosis; diabetes in women, which often leads to polycystic ovarian disease; and cardiovascular disease, which often leads to impotence. Accordingly, the compositions of the invention may be used for the prevention of one disease or disorder and concurrently treating another (e.g., prevention of polycystic ovarian disease while treating diabetes; prevention of impotence while treating a cardiovascular disease).

5.7.1. Cardiovascular Diseases for Treatment or Prevention

The present invention provides methods for the treatment or prevention of a cardiovascular disease, comprising administering to a patient a therapeutically effective amount of a compound or a composition comprising a compound of the invention and a pharmaceutically acceptable vehicle. As used herein, the term “cardiovascular diseases” refers to diseases of the heart and circulatory system. These diseases are often associated with dyslipoproteinemias and/or dyslipidemias. Cardiovascular diseases which the compositions of the present invention are useful for preventing or treating include but are not limited to arteriosclerosis; atherosclerosis; stroke; ischemia; endothelium dysfunctions, in particular those dysfunctions affecting blood vessel elasticity; peripheral vascular disease; coronary heart disease; myocardial infarcation; cerebral infarction and restenosis.

5.7.2. Dyslipidemias for Treatment or Prevention

The present invention provides methods for the treatment or prevention of a dyslipidemia comprising administering to a patient a therapeutically effective amount of a compound or a composition comprising a compound of the invention and a pharmaceutically acceptable vehicle.

As used herein, the term “dyslipidemias” refers to disorders that lead to or are manifested by aberrant levels of circulating lipids. To the extent that levels of lipids in the blood are too high, the compositions of the invention are administered to a patient to restore normal levels. Normal levels of lipids are reported in medical treatises known to those of skill in the art. For example, recommended blood levels of LDL, HDL, free triglycerides and others parameters relating to lipid metabolism can be found at the web site of the American Heart Association and that of the National Cholesterol Education Program of the National Heart, Lung and Blood Institute (http://www.americanheart.org/cholesterol/ about_level.html and http://www.nhlbi.nih.gov/health/ public/heart/chol/hbc_what.html, respectively). At the present time, the recommended level of HDL cholesterol in the blood is above 35 mg/dL; the recommended level of LDL cholesterol in the blood is below 130 mg/dL; the recommended LDL:HDL cholesterol ratio in the blood is below 5:1, ideally 3.5:1; and the recommended level of free triglycerides in the blood is less than 200 mg/dL.

Dyslipidemias which the compositions of the present invention are useful for preventing or treating include but are not limited to hyperlipidemia and low blood levels of high density lipoprotein (HDL) cholesterol. In certain embodiments, the hyperlipidemia for prevention or treatment by the compounds of the present invention is familial hypercholesterolemia; familial combined hyperlipidemia; reduced or deficient lipoprotein lipase levels or activity, including reductions or deficiencies resulting from lipoprotein lipase mutations; hypertriglyceridemia; hypercholesterolemia; high blood levels of ketone bodies (e.g. β-OH butyric acid); high blood levels of Lp(a) cholesterol; high blood levels of low density lipoprotein (LDL) cholesterol; high blood levels of very low density lipoprotein (VLDL) cholesterol and high blood levels of non-esterified fatty acids.

The present invention further provides methods for altering lipid metabolism in a patient, e.g., reducing LDL in the blood of a patient, reducing free triglycerides in the blood of a patient, increasing the ratio of HDL to LDL in the blood of a patient, and inhibiting saponified and/or non-saponified fatty acid synthesis, said methods comprising administering to the patient a compound or a composition comprising a compound of the invention in an amount effective alter lipid metabolism.

5.7.3. Dyslipoproteinemias for Treatment or Prevention

The present invention provides methods for the treatment or prevention of a dyslipoproteinemia comprising administering to a patient a therapeutically effective amount of a compound or a composition comprising a compound of the invention and a pharmaceutically acceptable vehicle.

As used herein, the term “dyslipoproteinemias” refers to disorders that lead to or are manifested by aberrant levels of circulating lipoproteins. To the extent that levels of lipoproteins in the blood are too high, the compositions of the invention are administered to a patient to restore normal levels. Conversely, to the extent that levels of lipoproteins in the blood are too low, the compositions of the invention are administered to a patient to restore normal levels. Normal levels of lipoproteins are reported in medical treatises known to those of skill in the art.

Dyslipoproteinemias which the compositions of the present invention are useful for preventing or treating include but are not limited to high blood levels of LDL; high blood levels of apolipoprotein B (apo B); high blood levels of Lp(a); high blood levels of apo(a); high blood levels of VLDL; low blood levels of HDL; reduced or deficient lipoprotein lipase levels or activity, including reductions or deficiencies resulting from lipoprotein lipase mutations; hypoalphalipoproteinemia; lipoprotein abnormalities associated with diabetes; lipoprotein abnormalities associated with obesity; lipoprotein abnormalities associated with Alzheimer's Disease; and familial combined hyperlipidemia.

The present invention further provides methods for reducing apo C-II levels in the blood of a patient; reducing apo C-III levels in the blood of a patient; elevating the levels of HDL associated proteins, including but not limited to apo A-I, apo A-II, apo A-IV and apo E in the blood of a patient; elevating the levels of apo E in the blood of a patient, and promoting clearance of triglycerides from the blood of a patient, said methods comprising administering to the patient a compound or a composition comprising a compound of the invention in an amount effective to bring about said reduction, elevation or promotion, respectively.

5.7.4. Glucose Metabolism Disorders for Treatment or Prevention

The present invention provides methods for the treatment or prevention of a glucose metabolism disorder, comprising administering to a patient a therapeutically effective amount of a compound or a composition comprising a compound of the invention and a pharmaceutically acceptable vehicle. As used herein, the term “glucose metabolism disorders” refers to disorders that lead to or are manifested by aberrant glucose storage and/or utilization. To the extent that indicia of glucose metabolism (i.e., blood insulin, blood glucose) are too high, the compositions of the invention are administered to a patient to restore normal levels. Conversely, to the extent that indicia of glucose metabolism are too low, the compositions of the invention are administered to a patient to restore normal levels. Normal indicia of glucose metabolism are reported in medical treatises known to those of skill in the art.

Glucose metabolism disorders which the compositions of the present invention are useful for preventing or treating include but are not limited to impaired glucose tolerance; insulin resistance; insulin resistance related breast, colon or prostate cancer; diabetes, including but not limited to non-insulin dependent diabetes mellitus (NIDDM), insulin dependent diabetes mellitus (IDDM), gestational diabetes mellitus (GDM), and maturity onset diabetes of the young (MODY); pancreatitis; hypertension; polycystic ovarian disease; and high levels of blood insulin and/or glucose.

The present invention further provides methods for altering glucose metabolism in a patient, for example to increase insulin sensitivity and/or oxygen consumption of a patient, said methods comprising administering to the patient a compound or a composition comprising a compound of the invention in an amount effective to alter glucose metabolism.

5.7.5. PPAR Associated Disorders for Treatment or Prevention

The present invention provides methods for the treatment or prevention of a PPAR-associated disorder, comprising administering to a patient a therapeutically effective amount of a compound or a composition comprising a compound of the invention and a pharmaceutically acceptable vehicle. As used herein, “treatment or prevention of PPAR associated disorders” encompasses treatment or prevention of rheumatoid arthritis; multiple sclerosis; psoriasis; inflammatory bowel diseases; breast; colon or prostate cancer; low levels of blood HDL; low levels of blood, lymph and/or cerebrospinal fluid apo E; low blood, lymph and/or cerebrospinal fluid levels of apo A-I; high levels of blood VLDL; high levels of blood LDL; high levels of blood triglyceride; high levels of blood apo B; high levels of blood apo C-III and reduced ratio of post-heparin hepatic lipase to lipoprotein lipase activity. HDL may be elevated in lymph and/or cerebral fluid.

5.7.6. Renal Diseases for Treatment or Prevention

The present invention provides methods for the treatment or prevention of a renal disease, comprising administering to a patient a therapeutically effective amount of a compound or a composition comprising a compound of the invention and a pharmaceutically acceptable vehicle. Renal diseases that can be treated by the compounds of the present invention include glomerular diseases (including but not limited to acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (including but not limited to acute tubular necrosis and acute renal failure, polycystic renal diseasemedullary sponge kidney, medullary cystic disease, nephrogenic diabetes, and renal tubular acidosis), tubulointerstitial diseases (including but not limited to pyclonephritis, drug and toxin induced tubulointerstitial nephritis, hypercalcemic nephropathy, and hypokalemic nephropathy) acute and rapidly progressive renal failure, chronic renal failure, nephrolithiasis, or tumors (including but not limited to renal cell carcinoma and nephroblastoma). In a most preferred embodiment, renal diseases that are treated by the compounds of the present invention are vascular diseases, including but not limited to hypertension, nephrosclerosis, microangiopathic hemolytic anemia, atheroembolic renal disease, diffuse cortical necrosis, and renal infarcts.

5.7.7. Cancers for Treatment or Prevention

The present invention provides methods for the treatment or prevention of cancer, comprising administering to a patient a therapeutically effective amount of a compound or a composition comprising a compound of the invention and a pharmaceutically acceptable vehicle. Cancers that can be treated or prevented by administering the compounds or the compositions of the invention include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In a most preferred embodiment, cancers that are treated or prevented by administering the compounds of the present invention are insulin resistance or Syndrome X related cancers, including but not limited to breast, prostate and colon cancer.

5.7.8. Other Diseases for Treatment or Prevention

The present invention provides methods for the treatment or prevention of Alzheimer's Disease, Syndrome X, septicemia, thrombotic disorders, obesity, pancreatitis, hypertension, inflammation, bacterial infection and impotence, comprising administering to a patient a therapeutically effective amount of a compound or a composition comprising a compound of the invention and a pharmaceutically acceptable vehicle.

As used herein, “treatment or prevention of Alzheimer's Disease” encompasses treatment or prevention of lipoprotein abnormalities associated with Alzheimer's Disease.

As used herein, “treatment or prevention of Syndrome X or Metabolic Syndrome” encompasses treatment or prevention of a symptom thereof, including but not limited to impaired glucose tolerance, hypertension and dyslipidemia/dyslipoproteinemia.

As used herein, “treatment or prevention of septicemia” encompasses treatment or prevention of septic shock.

As used herein, “treatment or prevention of thrombotic disorders” encompasses treatment or prevention of high blood levels of fibrinogen and promotion of fibrinolysis.

In addition to treating or preventing obesity, the compositions of the invention can be administered to an individual to promote weight reduction of the individual.

### 5.8. Surgical Uses

Cardiovascular diseases such as atherosclerosis often require surgical procedures such as angioplasty. Angioplasty is often accompanied by the placement of a reinforcing a metallic tube-shaped structure known as a “stent” into a damaged coronary artery. For more serious conditions, open heart surgery such as coronary bypass surgery may be required. These surgical procedures entail using invasive surgical devices and/or implants, and are associated with a high risk of restenosis and thrombosis. Accordingly, the compounds and compositions of the invention may be used as coatings on surgical devices (e.g., catheters) and implants (e.g., stents) to reduce the risk of restenosis and thrombosis associated with invasive procedures used in the treatment of cardiovascular diseases.

### 5.9. Veterinary and Livestock Uses

A composition of the invention can be administered to a non-human animal for a veterinary use for treating or preventing a disease or disorder disclosed herein.

In a specific embodiment, the non-human animal is a household pet. In another specific embodiment, the non-human animal is a livestock animal. In a preferred embodiment, the non-human animal is a mammal, most preferably a cow, horse, sheep, pig, cat, dog, mouse, rat, rabbit, or guinea pig. In another preferred embodiment, the non-human animal is a fowl species, most preferably a chicken, turkey, duck, goose, or quail.

In addition to veterinary uses, the compounds and compositions of the invention can be used to reduce the fat content of livestock to produce leaner meats. Alternatively, the compounds and compositions of the invention can be used to reduce the cholesterol content of eggs by administering the compounds to a chicken, quail, or duck hen. For non-human animal uses, the compounds and compositions of the invention can be administered via the animals' feed or orally as a drench composition.

### 5.10. Therapeutic/Prophylactic Administration and Compositions

Due to the activity of the compounds and compositions of the invention, they are useful in veterinary and human medicine. As described above, the compounds and compositions of the invention are useful for the treatment or prevention of cardiovascular diseases, dyslipidemias, dyslipoproteinemias, glucose metabolism disorders, Alzheimer's Disease, Syndrome X, PPAR-associated disorders, septicemia, thrombotic disorders, obesity, pancreatitis, hypertension, renal disease, cancer, inflammation, bacterial infection and impotence.

The invention provides methods of treatment and prophylaxis by administration to a patient of a therapeutically effective amount of a compound or a composition comprising a compound of the invention. The patient is an animal, including, but not limited, to an animal such a cow, horse, sheep, pig, chicken, turkey, quail, cat, dog, mouse, rat, rabbit, guinea pig, etc., and is more preferably a mammal, and most preferably a human.

The compounds and compositions of the invention, are preferably administered orally. The compounds and compositions of the invention may also be administered by any other convenient route, for example, by intravenous infusion or bolus injection, by absorption through epithelial or mucocutaneous linings (e.g., oral mucosa, rectal and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer a compound of the invention. In certain embodiments, more than one compound of the invention is administered to a patient. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner, and will depend in-part upon the site of the medical condition. In most instances, administration will result in the release of the compounds of the invention into the bloodstream.

In specific embodiments, it may be desirable to administer one or more compounds of the invention locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of an atherosclerotic plaque tissue.

In certain embodiments, for example, for the treatment of Alzheimer's Disease, it may be desirable to introduce one or more compounds of the invention into the central nervous system by any suitable route, including intraventricular, intrathecal and epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In another embodiment, the compounds and compositions of the invention can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compounds and compositions of the invention can be delivered in a controlled release system. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target area to be treated, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

The present compositions will contain a therapeutically effective amount of a compound of the invention, optionally more than one compound of the invention, preferably in purified form, together with a suitable amount of a pharmaceutically acceptable vehicle so as to provide the form for proper administration to the patient.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds and compositions of the invention and pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by E. W. Martin.

In a preferred embodiment, the compounds and compositions of the invention are formulated in accordance with routine procedures as a pharmaceutical composition adapted for intravenous administration to human beings. Typically, compounds and compositions of the invention for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of the invention is to be administered by intravenous infusion, it can be dispensed, for example, with an infusion bottle containing sterile pharmaceutical grade water or saline. Where the compound of the invention is administered by injection, an ampoule of sterile water for injection or saline can be provided so that the ingredients may be mixed prior to administration.

Compounds and compositions of the invention for oral delivery may be in the form of tablets, lozenges, aqueous or oily suspensions, granules, powders, emulsions, capsules, syrups, or elixirs. Compounds and compositions of the invention for oral delivery can also be formulated in foods and food mixes. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds and compositions of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

The amount of a compound of the invention that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for oral administration are generally about 0.001 milligram to 200 milligrams of a compound of the invention per kilogram body weight. In specific preferred embodiments of the invention, the oral dose is 0.01 milligram to 70 milligrams per kilogram body weight, more preferably 0.1 milligram to 50 milligrams per kilogram body weight, more preferably 0.5 milligram to 20 milligrams per kilogram body weight, and yet more preferably 1 milligram to 10 milligrams per kilogram body weight. In a most preferred embodiment, the oral dose is 5 milligrams of a compound of the invention per kilogram body weight. The dosage amounts described herein refer to total amounts administered; that is, if more than one compound of the invention is administered, the preferred dosages correspond to the total amount of the compounds of the invention administered. Oral compositions preferably contain 10% to 95% active ingredient by weight.

Suitable dosage ranges for intravenous (i.v.) administration are 0.01 milligram to 100 milligrams per kilogram body weight, 0.1 milligram to 35 milligrams per kilogram body weight, and 1 milligram to 10 milligrams per kilogram body weight. Suitable dosage ranges for intranasal administration are generally about 0.01 pg/kg body weight to 1 mg/kg body weight. Suppositories generally contain 0.01 milligram to 50 milligrams of a compound of the invention per kilogram body weight and comprise active ingredient in the range of 0.5% to 10% by weight. Recommended dosages for intradermal, intramuscular, intraperitoneal, subcutaneous, epidural, sublingual, intracerebral, intravaginal, transdermal administration or administration by inhalation are in the range of 0.001 milligram to 200 milligrams per kilogram of body weight. Suitable doses of the compounds of the invention for topical administration are in the range of 0.001 milligram to 1 milligram, depending on the area to which the compound is administered. Effective doses may be extrapolated from dose-response curves derived from in vitro or animal model test systems. Such animal models and systems are well known in the art.

The invention also provides pharmaceutical packs or kits comprising one or more containers filled with one or more compounds of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one compound of the invention. In another embodiment, the kit comprises a compound of the invention and another lipid-mediating compound, including but not limited to a statin, a thiazolidinedione, or a fibrate.

The compounds of the invention are preferably assayed in vitro and in vivo, for the desired therapeutic or prophylactic activity, prior to use in humans. For example, in vitro assays can be used to determine whether administration of a specific compound of the invention or a combination of compounds of the invention is preferred for lowering fatty acid synthesis. The compounds and compositions of the invention may also be demonstrated to be effective and safe using animal model systems.

Other methods will be known to the skilled artisan and are within the scope of the invention.

### 5.11. Combination Therapy

In certain embodiments of the present invention, the compounds and compositions of the invention can be used in combination therapy with at least one other therapeutic agent. The compound of the invention and the therapeutic agent can act additively or, more preferably, synergistically. In a preferred embodiment, a compound or a composition comprising a compound of the invention is administered concurrently with the administration of another therapeutic agent, which can be part of the same composition as the compound of the invention or a different composition. In another embodiment, a compound or a composition comprising a compound of the invention is administered prior or subsequent to administration of another therapeutic agent. As many of the disorders for which the compounds and compositions of the invention are useful in treating are chronic disorders, in one embodiment combination therapy involves alternating between administering a compound or a composition comprising a compound of the invention and a composition comprising another therapeutic agent, e.g., to minimize the toxicity associated with a particular drug. The duration of administration of each drug or therapeutic agent can be, e.g., one month, three months, six months, or a year. In certain embodiments, when a composition of the invention is administered concurrently with another therapeutic agent that potentially produces adverse side effects including but not limited to toxicity, the therapeutic agent can advantageously be administered at a dose that falls below the threshold at which the adverse side is elicited.

The present compositions can be administered together with a statin. Statins for use in combination with the compounds and compositions of the invention include but are not limited to atorvastatin, pravastatin, fluvastatin, lovastatin, simvastatin, and cerivastatin.

The present compositions can also be administered together with a PPAR agonist, for example a thiazolidinedione or a fibrate. Thiazolidinediones for use in combination with the compounds and compositions of the invention include but are not limited to 5-((4-(2-(methyl-2-pyridinylamino)ethoxy)phenyl)methyl)-2,4-thiazolidinedione, troglitazone, pioglitazone, ciglitazone, WAY-120,744, englitazone, AD 5075, darglitazone, and rosiglitazone. Fibrates for use in combination with the compounds and compositions of the invention include but are not limited to gemfibrozil, fenofibrate, clofibrate, or ciprofibrate. As mentioned previously, a therapeutically effective amount of a fibrate or thiazolidinedione often has toxic side effects. Accordingly, in a preferred embodiment of the present invention, when a composition of the invention is administered in combination with a PPAR agonist, the dosage of the PPAR agonist is below that which is accompanied by toxic side effects.

The present compositions can also be administered together with a bile-acid-binding resin. Bile-acid-binding resins for use in combination with the compounds and compositions of the invention include but are not limited to cholestyramine and colestipol hydrochloride. The present compositions can also be administered together with niacin or nicotinic acid. The present compositions can also be administered together with a RXR agonist. RXR agonists for use in combination with the compounds of the invention include but are-not limited to LG 100268, LGD 1069, 9-cis retinoic acid, 2-(1-(3,5,5,8,8-pentamethyl-5,6,7,8-tetrahydro-2-naphthyl)-cyclopropyl)-pyridine-5-carboxylic acid, or 4-((3,5,5,8,8-pentamethyl-5,6,7,8-tetrahydro-2-naphthyl)2-carbonyl)-benzoic acid. The present compositions can also be administered together with an anti-obesity drug. Anti-obesity drugs for use in combination with the compounds of the invention include but are not limited to β-adrenergic receptor agonists, preferably β-3 receptor agonists, fenfluramine, dexfenfluramine, sibutramine, bupropion, fluoxetine, and phentermine. The present compositions can also be administered together with a hormone. Hormones for use in combination with the compounds of the invention include but are not limited to thyroid hormone, estrogen and insulin. Preferred insulins include but are not limited to injectable insulin, transdermal insulin, inhaled insulin, or any combination thereof. As an alternative to insulin, an insulin derivative, secretagogue, sensitizer or mimetic may be used. Insulin secretagogues for use in combination with the compounds of the invention include but are not limited to forskolin, dibutryl cAMP or isobutylmethylxanthine (IBMX).

The present compositions can also be administered together with a tyrophostine or an analog thereof. Tyrophostines for use in combination with the compounds of the invention include but are not limited to tryophostine 51.

The present compositions can also be administered together with sulfonylurea-based drugs. Sulfonylurea-based drugs for use in combination with the compounds of the invention include, but are not limited to, glisoxepid, glyburide, acetohexamide, chlorpropamide, glibomuride, tolbutamide, tolazamide, glipizide, gliclazide, gliquidone, glyhexamide, phenbutamide, and tolcyclamide. The present compositions can also be administered together with a biguanide. Biguanides for use in combination with the compounds of the invention include but are not limited to metformin, phenformin and buformin.

The present compositions can also be administered together with an α-glucosidase inhibitor. α-glucosidase inhibitors for use in combination with the compounds of the invention include but are not limited to acarbose and miglitol.

The present compositions can also be administered together with an apo A-I agonist. In one embodiment, the apo A-I agonist is the Milano form of apo A-I (apo A-IM). In a preferred mode of the embodiment, the apo A-IM for administration in conjunction with the compounds of the invention is produced by the method of U.S. Pat. No. 5,721,114 to Abrahamsen. In a more preferred embodiment, the apo A-I agonist is a peptide agonist. In a preferred mode of the embodiment, the apo A-I peptide agonist for administration in conjunction with the compounds of the invention is a peptide of U.S. Pat. No. 6,004,925 or 6,037,323 to Dasseux.

The present compositions can also be administered together with apolipoprotein E (apo E). In a preferred mode of the embodiment, the apoE for administration in conjunction with the compounds of the invention is produced by the method of U.S. Pat. No. 5,834,596 to Ageland.

In yet other embodiments, the present compositions can be administered together with an HDL-raising drug; an HDL enhancer; or a regulator of the apolipoprotein A-I, apolipoprotein A-IV and/or apolipoprotein genes.

### 5.12. Combination Therapy with Cardiovascular Drugs

The present compositions can be administered together with a known cardiovascular drug. Cardiovascular drugs for use in combination with the compounds of the invention to prevent or treat cardiovascular diseases include but are not limited to peripheral antiadrenergic drugs, centrally acting antihypertensive drugs (e.g., methyldopa, methyldopa HCl), antihypertensive direct vasodilators (e.g., diazoxide, hydralazine HCl), drugs affecting renin-angiotensin system, peripheral vasodilators, phentolamine, antianginal drugs, cardiac glycosides, inodilators (e.g., amrinone, milrinone, enoximone, fenoximone, imazodan, sulmazole), antidysrhythmic drugs, calcium entry blockers, ranitine, bosentan, and rezulin.

### 5.13. Combination Therapy for Cancer Treatment

The present compositions can be administered together with treatment with irradiation or one or more chemotherapeutic agents. For irridiation treatment, the irradiation can be gamma rays or X-rays. For a general overview of radiation therapy, see Hellman, Chapter 12: Principles of Radiation Therapy Cancer, in: Principles and Practice of Oncology, DeVita et al., eds., 2^{nd}. Ed., J. B. Lippencott Company, Philadelphia. Useful chemotherapeutic agents include methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel. In a specific embodiment, a composition of the invention further comprises one or more chemotherapeutic agents and/or is administered concurrently with radiation therapy. In another specific embodiment, chemotherapy or radiation therapy is administered prior or subsequent to administration of a present composition, preferably at least an hour, five hours, 12 hours, a day, a week, a month, more preferably several months (e.g., up to three months), subsequent to administration of a composition of the invention.

### 5.14. Docking Procedures for the Identification of Non-Substrate Inhibitors of Acyl Coenzyme A Ligases and Acyl Coenzyme A Metabolizing Enzymes

The present invention is directed, in part, toward obtaining compounds useful for the prevention and treatment the conditions disclosed above. More specifically, the present invention is directed toward obtaining acyl coenzyme A mimics that are selective, non-substrate inhibitors of acyl coenzyme A ligases and acyl coenzyme A metabolizing enzymes. Identification of such inhibitors is carried out using computer-assisted methods including, but not limited to, docking procedures and the development and use of pharmacophore models.

In certain embodiments, the acyl coenzyme A metabolizing or binding proteins are acyl coenzyme A or fatty acid ligases. Exemplary acyl CoA ligases include, but are not limited to acetate--coA ligase (EC 6.2.1.1), butyrate--coA ligase (EC 6.2.1.2), long-chain-fatty-acid--coA ligase (EC 6.2.1.3), succinate--coA ligase (GDP-forming) (EC 6.2.1.4), succinate--coA ligase (ADP-forming) (EC 6.2.1.5), glutarate--coA ligase (EC 6.2.1.6), cholate--coA ligase (EC 6.2.1.7), oxalate--coA ligase (EC 6.2.1.8), malate--coA ligase (EC 6.2.1.9), acid--coA ligase (GDP-forming) (EC 6.2.1.10), biotin--coA ligase (EC 6.2.1.11), 4-coumarate--coA ligase (EC 6.2.1.12), acetate--coA ligase (ADP-forming) (EC 6.2.1.13), 6-carboxyhexanoate--coA ligase (EC 6.2.1.14), arachidonate--coA ligase (EC 6.2.1.15), acetoacetate--coA ligase (EC 6.2.1.16), propionate--coA ligase (EC 6.2.1.17), citrate--coA ligase (EC 6.2.1.18), long-chain-fatty-a cid--luciferin-component ligase (EC 6.2.1.19), long-chain-fatty-acid--acyl-carrier protein ligase (EC 6.2.1.20), [citrate (pro-3S)-lyase] ligase (EC 6.2.1.22), dicarboxylate--coA ligase (EC 6.2.1.23), phytanate--coA ligase (EC 6.2.1.24), benzoate--coA ligase (EC 6.2.1.25), O-succinylbenzoate--coA ligase (EC 6.2.1.26), 4-hydroxybenzoate--coA ligase (EC 6.2.1.27), 3-alpha,7-alpha-dihydroxy-5-beta-cholestanate--coA ligase (EC 6.2.1.28), 3-alpha,7-alpha, 12-alpha-trihydroxy-5-beta-cholestanate--coA ligase (EC 6.2.1.29), phenylacetate--coA ligase (EC 6.2.1.30), 2-furoate--coA ligase (EC 6.2.1.31), anthranilate--coA ligase (EC 6.2.1.32), 4-chlorobenzoate-coA ligase (EC 6.2.1.33), and trans-feruloyl-coA synthase (EC 6.2.1.34). Methods of isolation and/or determining binding to and/or measuring activity of an acyl coenzyme A ligase are described in Aas and Bremer, 1968, Biochim Biophys Acta 164(2):157-66; Barth et al., 1971, Biochim Biophys Acta 248(1):24-33; Groot, 1975, Biochim Biophys Acta 380(1):12-20; Scholte et al., 1971, Biochim Biophys Acta 231(3):479-86; Scholte and Groot, 1975, Biochim Biophys Acta 409(3):283-96; Scaife and Tichivangana, 1980, Biochim Biophys Acta. 619(2):445-50; Man and Brosnan, 1984, Int J Biochem. 1984;16(12):1341-3; Patel and Walt, 1987, J Biol Chem. 262(15):7132-4; Philipp and Parsons, 1979, J Biol Chem. 254(21):19785-90; Vanden Heuvel et al., 1991, Biochem Pharmacol. 42(2):295-302; Youssef et al., 1994, Toxicol Lett. 74(1):15-21; and Vessey et al., 1999, Biochim Biophys Actal428(2-3):455-62.

In other embodiments, the acyl coenzyme A metabolizing or binding proteins are enzymes or proteins involved in reactions utilizing acyl carrier protein (ACP). Exemplary ACPs include, but are not limited to, [acyl-carrier-protein] acetyltransferase (EC 2.3.1.38), [acyl-carrier-protein] malonyltransferase (EC 2.3.1.39), [acyl-carrier-protein] phosphodiesterase (EC 3.1.4.14); enoyl-[acyl-carrier-protein] reductase (NADPH) (EC 1.3.1.10), holo-[acyl-carrier-protein] synthase (EC 2.7.8.7), 3-oxoacyl-enzyme [acyl-carrier protein], 3-oxoacyl-[acyl-carrier-protein] reductase (EC 1.1.1.100 ), or 3-oxoacyl-[acyl-carrier-protein] synthase (EC 2.3.1.41).

In yet other embodiments, the acyl coenzyme A metabolizing or binding proteins are enzymes or proteins involved in reactions using Coenzyme A. Exemplary enzymes or proteins involved in reactions using Coenzyme A include, but are not limited to, acetate-coA ligase (EC 6.2.1.1), acetoacetyl-coA hydrolase (EC 3.1.2.11), acetoacetyl-coA: acetate coA transferase (EC 2.8.3.8 ), acetyl-coA acetyltransferase [thiolase] (EC 2.3.1.9), acetyl-coA acyltransferase (EC 2.3.1.16), acetyl-coA carboxylase (EC 6.4.1.2), [acetyl-coA carboxylase] phosphatase (EC 3.1.3.4), acetyl-coA ligase (EC 6.2.1.1), acyl-coA acyltransferase (EC 2.3.1.16), acyl-coA dehydrogenase (EC 1.3.99.3), acyl-coA dehydrogenase (NADP+) (EC 1.3.1.8), butyryl-coA dehydrogenase (EC 1.3.99.2), cholate-coA ligase (EC 6.2.1.7), dephospho-coA kinase (EC 2.7.1.24), enoyl-coA hydratase (EC 4.2.1.17), formyl-coA hydrolase (EC 3.1.2.10), glucan-1,4-a-glucosidase [glucoAmylase] (EC 3.2.1.3), glutaryl-coA dehydrogenase (EC 1.3.99.7), glutaryl-coA ligase (EC 6.2.1.6), 3-hydroxyacyl-coA dehydrogenase (EC 1.1.1.35), 3-hydroxybutyryl-coA dehydratase (EC 4.2.1.55), 3-hydroxybutyryl-coA dehydrogenase (EC 1.1.1.157), 3-hydroxyisobutyryl-coA hydrolase (EC 3.1.2.4), hydroxymethylglutaryl-coA lyase (EC 4.1.3.4), hydroxymethylglutaryl-coA reductase (EC 1.1.1.88), hydroxymethylglutaryl-coA reductase (NADPH) (EC 1.1.1.34), [hydroxymethylglutaryl-coA reductase (NADPH)] kinase (EC 2.7.1.109), [hydroxymethylglutaryl-coA reductase (nadph)] phosphatase (EC 3.1.3.47), hydroxymethylglutaryl-coA synthase (EC 4.1.3.5), lactoyl-coA dehydratase (EC 4.2.1.54), malonate-coA transferase (EC 2.8.3.3), malonyl-coA decarboxylase (EC 4.1.1.9), methylcrotonyl-coA carboxylase (EC 6.4.1.4), methylglutaconyl-coA hydratase (EC 4.2.1.18), methylmalonyl-coA carboxyltransferase (EC 2.1.3.1), methylmalonyl-coA decarboxylase (EC 4.1.1.41), methylmalonyl-coA epimerase (EC 5.1.99.1), methylmalonyl-coA mutase (EC 5.4.99.2), oxalate-coA transferase (EC 2.8.3.2), oxalyl-coA decarboxylase (EC 4.1.1.8), 3-oxoacid-coA transferase (EC 2.8.3.5), 3-oxoadipate coA-transferase (EC 2.8.3.6), palmitoyl-coA-enzyme palmitoyltransferase, propionate-coA ligase (EC 6.2.1.17), propionyl-coA carboxylase (EC 6.4.1.3), succinate-coA ligase (ADP-forming) (EC 6.2.1.5), succinate-coA ligase (GDP-forming) (EC 6.2.1.4), or succinate-propionate coA transferase.

In yet other embodiments, the acyl coenzyme A metabolizing or binding proteins are enzymes or proteins involved in reactions resulting in the biosynthesis or degradation of coA. Exemplary enzymes or proteins involved in reactions resulting in the biosynthesis or degradation of coA include, but are not limited to, pantothenatekinase (EC 2.7.1.33), pantothenate-B-alanine ligase (EC 6.3.2.1), phosphopantothenate-cysteine ligase (EC 6.3.2.5), pantetheine kinase (EC 2.7.1.34), pantetheine-phosphate adenylyltransferase (EC 2.7.7.3), 2-dehydropantoate reductase (EC 1.1.1.169), pantothenase (EC 3.5.1.22), pantothenoylcysteine decarboxylase (EC 4.1.1.30), phosphopantothenate-cysteine ligase (EC 6.3.2.5), phosphopantothenoylcysteine decarboxylase (EC 4.1.1.36).

In yet other embodiments, the acyl coenzyme A metabolizing or binding proteins are enzymes or proteins involved in the “mevalonate shunt,” as described in Edmond and Popjak, 1974, J. Biol. Chem. 249:66-71

In specific embodiments, the present invention is directed toward obtaining acyl coenzyme A mimics that are selective, non-substrate inhibitors of short-chain acyl coenzyme A ligases and of short-chain acyl coenzyme A metabolizing enzymes.

Docking procedures involve inter alia the computer-assisted determination and evaluation of the interaction between a biological macromolecule and a ligand. In certain embodiments, the biological macromolecule is an enzyme and the ligand may be a substrate, or a non-substrate inhibitor, of that enzyme. Non-substrate inhibitors can be, but are not limited to, structural analogs or molecular mimics, in whole or in part, of a natural substrate of the enzyme. Accordingly, docking procedures are used in the present invention both qualitatively and quantitatively for the identification of putative inhibitors of, e.g., short-chain acyl coenzyme A ligases and of short-chain acyl coenzyme A metabolizing enzymes. Such docking procedures are also used to evaluate the binding of those putative identified inhibitors to long-chain acyl coenzyme A ligases and long-chain acyl coenzyme A metabolizing enzymes. Comparison of the relative binding strength of the identified, putative inhibitors to each class of acyl coenzyme A binding enzyme provides an indication of the specificity and selectivity of the inhibitor.

The docking procedures of the present invention employ computation tools for the identification and evaluation of energetically favorable binding interactions between a biological macromolecule and a ligand that have been shown to be useful for structure-based drug design, such as those disclosed in U.S. Pat. Nos. 5,866,343, 6,341,256 B1, and 6,365,626 B1, each of which is hereby incorporated by reference in its entirety. The docking approaches useful in different aspects of the present invention fall into two main categories, namely, qualitative and quantitative methods. Qualitative methods are restricted primarily to calculations based on shape, complementarity and consist of finding the best fit between two shapes, which can be carried out, in one non-limiting approach, using the computer program called “Dock,” as described B. K. Shoichet et al. (Shichet et al., Protein Engineering, 7: 723-732, 1993, which is hereby incorporated by reference in its entirety). Quantitative methods useful in the docking methods of the present invention are based primarily on energy calculations designed to detennine the global minimum energy of the ligand binding interaction with the protein target. One non-limiting description of a method useful in this aspect of the invention is provided by Kollman (Kollam, Chem. Rev. 93: 2395-2417, 1993, which is hereby incorporated by reference in its entirety). Moreover, the docking methods of the present invention further comprise hybrid methods in which an interaction energy is calculated for the binding of a target protein and an individual fragment of a putative ligand; the resulting data are then assembled based on shape, complementarity criteria to form new ligand molecules. This aspect of the present invention uses, in one non-limiting example, the approach described by P. A. Goodford (Goodford, J. Med. Chem, 28: 849-857, 1985, which is hereby incorporated by reference in its entirety).

By using the docking methods of the present invention, intermolecular movement between the biological macromolecule and ligand are simulated by computing intermolecular forces to evaluate preferred “docking” interactions between the molecules. According to these methods, the energy of the interaction between the two molecules is calculated in order to define, as the best binding site interactions, those which have the most favorable or minimum potential energy. That is, it is possible to rank a series of putative ligands with respect to their relative ability to bind to the biological macromolecule. Moreover, therefore, it is also possible to compare the strength of the interaction of a given ligand with two different biological macromolecules, e.g., a short-chain acyl coenzyme A ligase and a long-chain acyl coenzyme A ligase. It should be noted that the predictive accuracy of any such quantitative method is limited by the resolution or precision of the model. In most calculations of such binding interactions, the molecular structures are mapped onto a grid. This mapping is performed either with or without a transfer function, e.g. a 1/r-function in the case of electrostatic potential description. The calculation of the interaction between the two biological macromolecule and the ligand, such as calculating the potential energy between the two molecules, is performed for each relative position of the two molecules, namely, each relative translational position and each rotational orientation between the two molecules.

In a preferred embodiment, therefore, the docking methods of the present invention make use of correlation between a potential grid, which represents one molecule, and an interaction field grid, which represents the second molecule, to obtain for each selected relative rotation between the two molecules, a potential energy that represents a binding energy of the two molecules for relative translational positions in space between the two molecules. Therefore, by using a single complex correlation calculation for each relative rotation between the two molecules, the resulting grids can be scanned to obtain the most energetically favorable binding interaction between two molecules. More specifically, by using a grid resolution in the range of 0.25 Å-0.45Å, this approach provides very acceptable quantitative results for determining molecule binding energy for all relative translational positions in space between the two molecules.

Therefore, in one embodiment the present invention, docking methods are employed that provide a quantitative value for an energetically favorable binding interaction between two molecules, i.e. a biological macromolecule and a ligand. In a specific embodiment of the present invention, the biological macromolecule is involved in the synthesis and or metabolism of an acyl coenzyme A compound while the ligand is an acyl coenzyme A mimic that binds to and/or inhibits the enzyme. One such method comprises the steps of: a) obtaining potential energy structural data for each atom site in the molecules; b) selecting a grid resolution corresponding to a sampling grid size substantially smaller than an average distance between bonded atoms in the molecules; c) selecting a range of relative rotations between the two molecules; d) mapping a plurality of potential energy field components of one of the molecules onto a corresponding one of a plurality of energy field component grids having the resolution with one molecule at a predetermined rotation and position, wherein each grid point of the component grids has a potential energy value interpolated from the potential energy structural data; e) mapping a plurality of interaction field components of another of the molecules onto a corresponding one of a plurality of interaction component grids having the resolution with the other molecule at a predetermined rotation and position, the interaction component corresponding to coefficients of a forcefield between the molecules, wherein each grid point of the component grids has an interaction value interpolated from the potential energy structural data; f) calculating a correlation between each potential energy field component grid and each interaction field component grid to obtain a grid of molecule binding energy values representing a binding energy of the two molecules in the relative rotation for relative translational positions in space between the molecules; g) determining at least one maximum of the binding energy values and recording the relative translational positions for the maximum binding energy values; h) rotating at least one of the molecules according to each relative rotation in the range, repeating the step of mapping for the at least one of the molecules and subsequently repeating the steps (f) and (g) of calculating and determining for each relative rotation; and i) selecting an energetically favorable one of the relative rotations in the range and the relative translational positions based on the maximum binding energy values to generate the position value for an energetically favorable binding site between the two molecules.

Therefore, according this method, only one molecule, e.g., the biological macromolecule, needs to be rotated relative to the other, e.g. the ligand which is a putative inhibitor of the biochemical activity of the biological macromolecule. Consequently, the map of one of the molecules can be used repeatedly while the map of the second molecule can be recalculated for each new rotational position. That is, the map of the target macromolecule can be used repeatedly, while that for each ligand/putative inhibitor is varied. Since the interaction field components are easier to map, it is preferred that only the interaction component grids be remapped for each new rotation. Also preferably, the preferred transform for carrying out the correlation is the discrete Fourier transform.

Preferably, the potential energy field components consist of the electrostatic potential which is based on Coulomb's law and varies as a function of 1/r, a second component for the first Van der Waals term A, which varies as a function of 1/r¹² and a third component for the second Van der Waals term B, which varies as a function of 1/r⁶. The result of the correlation for each field component must be summed with the results of the other components in order to obtain a total binding energy of the two molecules for the given relative rotation and for each relative translational position in space provided within the grid.

The docking methods of the present invention are directed toward obtaining and evaluating interactions between ligands, which may be non-substrate inhibitors, and biological macromolecules which are proteins, and more specifically, are short-chain acyl coenzyme A ligases, long-chain acyl coenzyme A ligases, short-chain acyl coenzyme A metabolizing enzymes, and long-chain acyl coenzyme A metabolizing enzymes. The potential energy of the system consisting of the protein and ligand is calculated by determining the potential energy field created by the protein and then calculating the potential energy resulting from the contribution of each atom in the ligand for a particular position in space within the potential energy field of the protein. The potential energy is calculated using three basic terms. The first term is the electrostatic potential. This results from an electrostatic charge at a particular atom within the ligand interacting with the electrostatic field potential created by the molecule. Such potentials are greater in polar or ionic molecules. The second and third potential energy terms come from the Van der Waals potentials, which is generally the 6-12 Lennard Jones potential. The combination of the three potential energy terms are used to provide a potential energy minimum (maximum binding energy) as a particular radial distance. Potential terms can be extended by an explicit term for hydrogen bond interaction, using, as one non-limiting example, the methods and approaches disclosed in U.S. Pat. Nos. 5,642,292, and 6,308,145 B1, each of which is hereby incorporated by reference in its entirety.

For the chosen protein and the chosen ligand/putative inhibitor, data concerning static charge at the atom sites in the molecules as well as the coefficients for the Van der Waals forces are obtained from existing databases. Such potential energy structural data is originally determined empirically and/or by theoretical model calculations. Next, a grid resolution corresponding to a sampling grid size substantially smaller than an average distance between atoms in the molecules is selected. A sampling grid size of 0.4 Å provides, in most cases, sufficiently high resolution to obtain good results for protein ligand pairs. A grid resolution of 0.25Å, while computationally more intensive, provides substantially more accurate results.

Once the grid resolution is selected, each potential energy field component of one of the molecules, in the preferred embodiment the protein, is mapped onto a corresponding energy field component grid. This typically involves calculating for each grid point the potential energy field created by each atom site in the protein and summing all potentials to obtain the field potential. Since this step of mapping may only be carried out once for each target protein, the effect of every atom site in the protein may be taken into account and all of the computation time required may be taken. For atoms very close to a grid point, where computational errors can result from selection outside the representation range of numbers in a computer, an arbitrary high value for their contribution to the potential field is taken. The relative spatial coordinates of each atom site for the protein and for the ligand are known from the structural data obtained from existing databases, or from predicted structural data.

The ligands, which can be non-substrate inhibitors of the enzymes indicated above, are generally much smaller molecule and therefore are easier to map onto the grid. The potential energy field components are not mapped onto the grid but rather the interaction field components are mapped onto the grid. The interaction field components relate to the charge quantities in the case of the electrostatic potential and the Van der Waals coefficients in the case of the Van der Waals potentials. For each atom site, the coefficients associated therewith are mapped onto the grid points surrounding each atom site in virtual space. The interpolation method for such mapping may be trilinear or a Gaussian distribution. Calculation of the values for the interaction field grid relating to the ligand involves carrying out a series of simple calculations with respect to each atom site in the ligand. The interaction component grids are built up for the particular rotational orientation of the ligand within the grid space by calculating the interaction field components for all of the atom sites in the ligand.

Since the potential energy field grids and the corresponding interaction field component grids have the same grid resolution and grid size, a correlation between the two grids may be calculated. In a preferred embodiment, the discrete Fourier transform using a fast Fourier transform method is applied to each grid. The two transformed grids are then multiplied using element by element multiplication to obtain an intermediate product grid, and then the intermediate product grid is subjected to an inverse fast Fourier transform to obtain a grid representing for each point in the grid a binding energy for each component for each translational position in space between the protein and the ligand. By summing the resulting component grids for the binding energies, a single total binding energy grid is obtained. The total binding energy grid is scanned to determine a maximum binding energy value for the particular rotation of the ligand. As can also be appreciated, if an atom site happens to fall directly on a grid point as a result of the virtual rotation, the computational accuracy is not compromised. For this reason, it is further preferred to rotate the molecule whose interaction field components are being calculated and mapped onto the grid rather than rotating the molecule whose potential energy field components are being mapped. The method described thus far is carried out for every conceivable relative rotation between the protein and the ligand. Since, in many cases, the ligands/putative inhibitors of the present invention are structural analogs or molecular mimics, in whole or in part, of coenzyme, A, and the interaction between the enzyme and coenzyme A may have been previously characterized, not all possible orientations need be examined.

Since, generally, only a small part of the protein will adjust to a different conformation on the incoming ligand, potential energy components are then preferably mapped in two parts. First the potential energy field grid is mapped for the larger part of the protein which does not change conformation, and this first grid is stored and reused each time. To calculate the total potential energy field grid for each conformation of the protein, the potential energy grid for the second part of the protein, which has assumed a different conformation, is calculated. The potential energy field grid of the first part is added to the potential energy grid of the second part to obtain the total potential energy field grid for the protein in the conformational state. This method of mapping the potential energy component grids is preferred because the computational time required to map the potential energy components onto the component grids is significant for larger molecules.

In one embodiment of, the docking methods of the present invention are applied using, as the biological macromolecular component of the interaction, a short-chain acyl coenzyme A ligase, such as but not limited to a short chain acyl coenzyme A synthetase or butyrate-CoA ligase. In another embodiment, the biological macromolecular component of the interaction, is a short-chain acyl coenzyme A metabolizing enzyme selected from the group consisting of aceto acetyl-CoA thiolase, HMG-CoA synthase, and HMG-CoA reductase. In each of these embodiments, putative inhibitors, which are ligands identified by virtue of the computed binding energy of their interaction with the biological macromolecule examined, are docked, using the same methods to one or more long-chain acyl coenzyme A ligases and/or one or more long-chain acyl coenzyme A metabolizing enzymes, such as, but not limited to those selected from the group consisting of fatty acyl CoA synthetase and palymitoyl CoA synthetase long chain acyl-CoA oxidase, long-chain enoyl-CoA hydratase, and long chain hydoxyacyl CoA dehydrogenase.

In another embodiment of the present invention, which is particularly useful for screening purposes for obtaining non-substrate inhibitors useful for treatment of the conditions disclosed above, a consensus three-dimensional structure is constructed for each of the following enzymes: (a) short-chain acyl coenzyme A ligase, (b) a short-chain acyl coenzyme A ligase, (c) a long-chain acyl coenzyme A ligase, and (d) a long-chain acyl coenzyme A metabolizing enzyme. The construction of such consensus structures is facilitated by the existence of publically-avail able crystal structures for representative enzymes. Moreover, since these structures include complexes of the enzyme and substrate, conformational alterations resulting from substrate binding, as well as the delineation of the substrate-binding site, and amino acid residues involved in and/or critical to that binding, may be inferred by those skilled in the art. See for example, the structures provided by the Protein Data Bank (http://rutgers.rcsb.org/pdb) and described by Berman et al. (Berman et al. 2000, Nucleic Acids Research 28(1): 235-42).

For example, such a consensus structure may be constructed by superimposing the coordinates each of the crystal structures that are publically available using the InsightII computer program ((1996), Molecular Simulations, Inc., San Diego, Calif.) to provide the best overall structural comparison, in which each of the input amino acid sequences are aligned based on the superimposition of their structures. Such sequence alignment accommodates such features as loops in a protein which differ from the other protein sequences. The structural superimposition is performed using the Homology module of the InsightI ((1996), Molecular Simulations, Inc., San Diego, Calif.) program and, in one non-limiting example, a Silicon Graphics INDIGO2 computer (Silicon Graphics Inc., Mountain View, Calif.). The sequence alignment can be manually adjusted and sequence variation profile can be provided for each input amino acid sequence. The sequence variation profile can then be used for comparing the consensus structure so determined with each new protein to be examined. In this procedure, the sequence of a target protein is read into the program and manually aligned with the known proteins based on the sequence variation profile described previously. A set of three-dimensional coordinates can then be assigned to a target protein using the Homology module of the InsightII program ((1996), Molecular Simulations, Inc., San Diego, Calif.). The coordinates for loop regions resulting, e.g. in a new, target protein, resulting from an insertion a number of amino acids, can be automatically generated by the computer program and manually adjusted to provide a more ideal geometry using the program CHAIN (Sack, J. S. (1988) J. Mol. Graphics 6, 244-245). Finally, the molecular model derived for the new target protein is subjected to energy minimization using the X-plor program (Brunger, A. T. (1992), New Haven, Ct.) so that any steric strain introduced during the model-building process is be relieved. Such a model can then be screened for unfavorable steric contacts and if necessary such side chains were remodeled either by using a rotamer library database or by manually rotating the respective side chains. A molecular structure constructed in this manner can then be used in the docking procedures described above to obtain the desired inhibitors.

If the three dimensional structure of a ligand is not known, one can use one or more computer programs, including but not limited to, CATALYST (Molecular Simulations, Inc., San Diego, Calif.), to predict the three-dimensional structure of the compound. Three-dimensional conformers are generated from a starting structure using software well known in the art such as, but not limited to, the Best or Fast Conformational Analyses (Molecular Simulations, Inc., San Diego, Calif.). In addition, where the ligand or putative inhibitor is a structural analog or molecular mimic of all or part of a natural substrate of the target enzyme, the three-dimensional structure of that substrate can be used to predict the three-dimensional structure of the subject ligand. This is particularly helpful where the three-dimensional structure of the natural substrate has been established by X-ray crystallography of an enzyme-substrate complex.

In one embodiment, analysis of such is carried out using the Docking module within the program INSIGHTII and using the Affinity suite of programs for automatically docking a ligand to the biological macromolecule i.e. enzyme. As notes above, hydrogen atoms on the ligand and enzyme are generated and potentials are assigned to both enzyme and ligand prior to the start of the docking procedure. The docking method in the InsightIl program uses the CVFF force field and a Monte Carlo search strategy to search for and evaluate docked structures. While the coordinates for the bulk of the receptor are kept fixed, a defined region of the substrate-binding site is allowed to relax, thereby permitting the protein to adjust to the binding of different inhibitors. A binding set is defined within a distance of 5 Å from the inhibitor, allowing residues within this distance to shift and/or rotate to energetically favorable positions to accommodate the ligand. An assembly is defined consisting of the receptor and inhibitor molecule and docking performed using the fixed docking mode. Calculations approximating hydrophobic and hydrophilic interactions are used to determine the ten best docking positions of each ligand enzyme's substrate-binding site. The various docked positions of ligand are qualitatively evaluated using Ludi (Bohm, H. J. (1992) J. Comput. Aided Mol. Des. 6(6): 593-606; and Bohm, H. J. (1994) J. Comput. Aided Mol. Des. 8(3): 243-56) in INSIGHTII which can be used to estimate a binding constant (Kₛ) for each compound in order to rank their relative binding capabilities and predicted inhibition of the target enzyme examined. The Kᵢ trends for ligands are compared with the trend of experimentally determined ligands/inhibitors in order to elucidate the structure-activity relationships (SAR) determining the potency of the ligands/inhibitors tested.

In another aspect of the present invention, the three-dimensional structure of the target enzyme, and more particularly, the substrate-binding site of that enzyme is inferred by comparing the amino acid sequence of that target protein to a homolog for which a crystal structure has been determined. In a still further aspect of the present invention, the three-dimensional structure of the target enzyme, and more particularly, the substrate-binding site of that enzyme, is determined by determining the structure using X-crystallography, NMR, or a combination of such methods, that are well known in the art.

### 5.15 Pharmacophore Models and Use thereof for the Identification of Non-Substrate Inhibitors of Short-Chain Acyl Coenzyme A Ligases and Short-Chain Acyl Coenzyme A Metabolizing Enzymes

In yet another aspect of the present invention, the structure of the target enzyme is not determined a priori. Rather, desired compounds, which are non-substrate inhibitors of short-chain acyl coenzyme A ligases and/or short-chain acyl coenzyme A metabolizing enzymes but are not inhibitors of long-chain acyl coenzyme A ligases and/or long-chain acyl coenzyme A metabolizing enzymes, are identified by constructing one or more pharmacophore models and then using those models to search databases of three-dimensional structures for compounds corresponding to the pharmaocophore. Compounds identified in this manner may then be used in the docking methods described above, or as lead compounds for the design and synthesis of inhibitors that may be tested in animal model systems, tissue extracts, or in vitro assay systems using purified enzymes, as disclosed herein. Methods useful for the construction and use of a pharmacophore model for the identification of ligands/inhibitors that bind target biological macromolecules are described in U.S. Pat. No. 6,365,626 B1, which is hereby incorporated by reference in its entirety.

Pharmacophore models are used to describe compounds on the basis of shared chemical features among identified inhibitors that are inferred to be critical to the binding interactions between the ligand/inhibitor and the chemical substructures within the substrate-binding site of the protein (e.g. see Tomioka et al., (1994) J. Comput. Aided. Mol. Des. 8(4): 347-66; Greene et al. (1994) J. Chem. Inf. Comput. Sci. 34: 1297-1308).

Accordingly, compounds useful in the methods of the present invention for the prevention and treatment of the conditions disclosed herein are identified in certain embodiments using computer-assisted methods that detect potential acyl CoA mimics that are selective inhibitors of enzymes forming and/or metabolizing short chain acyl CoA compounds. Such methods can comprise accessing a database of compounds which contains structural information for the compounds in the database and comparing the compounds in the database with a pharmacophore to obtain compounds having the features common to a collection of known acyl coenzyme A mimics that are selective inhibitors of short chain acyl coenzyme A formation and/or metabolism.

Such structural comparisons can be carried out using the software described above, generally using the default parameters supplied by the manufacturer. Such parameters, however, can be modified where desired. The number of hits to be found in a given database may be influenced by the nature of the pharmacophore or query structure used, the software employed, and the constraints applied to the searches performed by that software.

The computer-assisted methods used in combination with the pharmacophores described above provide those skilled in the art with a tool for obtaining compounds that can then be evaluated for activity, either in vivo or in vitro, using the assay systems disclosed herein. For example, those skilled in the art can use pharmacophores in conjunction with a computational computer program, such as CATALYST (Molecular Simulations, Inc., San Diego, Calif.), to search databases of existing compounds for compounds that fit a derived pharmacophore and that have the desired inhibitory activity. The degree of fit of an experimental compound structure to a pharmacophore is calculated using computer-assisted methods to determine whether the compound possesses the chemical features of the pharmacophore and whether the features can adopt the necessary three-dimensional arrangement to fit the model. The computer output provides information regarding those features of the pharmacophore that are fit by an experimental compound. A compound “fits” the pharmacophore if it has the features of the pharmacophore.

Computer programs useful for searching databases of chemical compounds useful in the methods of the present invention include ISIS (MDL Information Systems, Inc., San Leandro, Calif.), SYBYL (Tripos, Inc., St. Louis, Mo.), INSIGHT II (Pharmacopeia, Inc., Princeton, N.J.), and MOE (Chemical Computing Group, Inc., Montreal, Quebec, Canada). Examples of databases of chemical compounds that can be searched using such structure-recognition software include, but are not limited to the BioByte MasterFile (BioByte Corp., Claremont, Calif.), NCI (Laboratory of Medicinal Chemistry, National Cancer Institute, NIH, Frederick, Md.), Derwent (Derwent Information, London, UK) and Maybridge (Maybridge plc, Trevillett, Tintagel, Cornwall, UK) databases, which are available from Pharmacopeia, Inc., Princeton, N.J.). Software-assisted searches of chemical databases for compounds of the present invention can be performed using a wide variety of computer workstations or general purpose computer systems.

### 5.16. Biological Methods of Identifying Acyl Coenzyme a Mimics

The present invention provides biological assays for obtaining and identifying acyl coenzyme A mimics that are useful for treating or preventing a condition of the invention.

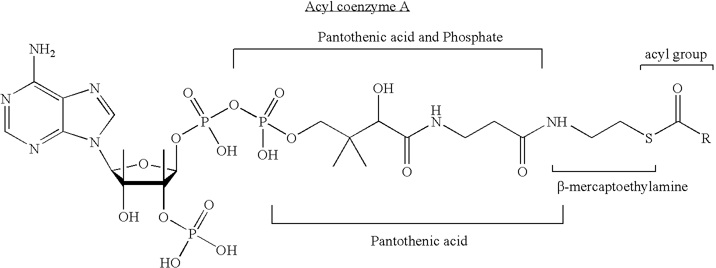

Without being bound by any theory, the present inventors believe that acyl coenzyme A mimics that bind to and/or inhibit the activity of acyl coenzyme A metabolizing or binding proteins are useful in treating or preventing diseases of the invention. As used herein the phrase “acyl coenzyme A mimic” also includes compounds that are mimics and analogs of coenzyme A as well as analogs of portions of coenzyme A, such as but not limited to the pantothenic acid portion of coenzyme A, including, but not limited to phosphorylated derivatives of pantothenic acid and analogs thereof.

Methods of measuring the binding or inhibition of acyl coenzyme A metabolizing or binding proteins by an acyl coenzyme A mimic are well known in the art. In certain embodiments, said binding or inhibition is measured by high pressure liquid chromatography, thin layer chromatography, mass spectrometry. The assays can be carried out on cellular extracts containing the acyl coenzyme A metabolizing or binding proteins or on purified, for example recombinantly expressed, acyl coenzyme A metabolizing or binding proteins.

In a preferred embodiment, the acyl coenzyme A mimic is a competitive inhibitor of acyl coenzyme A, and is most preferably a competitive inhibitor of acetyl coenzyme A. To determine whether a coenzyme A mimic is a competitive inhibitor of coenzyme A, the binding of the mimic to a fatty acid ligase is determined at two different concentrations of acyl coenzyme A. Compounds whose binding to the ligase is reduced at greater concentrations of acyl coenzyme A are competitive inhibitors of acyl coenzyme A. In other embodiments, the acyl coenzyme A mimic is a non-competitive inhibitor of acyl coenzyme A, preferably of acetyl coenzyme A. In yet other embodiments, the acyl coenzyme A mimic is an allosteric inhibitor of acyl coenzyme A, preferably of acetyl coenzyme A.

Test compounds that can be used in the present methods can include any compound from any source, including but not limited to compound libraries. The compounds can assayed singly or in multiplex format assays.

In certain embodiments, the acyl coenzyme A metabolizing or binding proteins are acyl coenzyme A or fatty acid ligases. Exemplary acyl CoA ligases include, but are not limited to acetate--CoA ligase (EC 6.2.1.1), butyrate--CoA ligase (EC 6.2.1.2), long-chain-fatty-acid--CoA ligase (EC 6.2.1.3), succinate--CoA ligase (GDP-forming) (EC 6.2.1.4), succinate--CoA ligase (ADP-forming) (EC 6.2.1.5), glutarate--CoA ligase (EC 6.2.1.6), cholate--CoA ligase (EC 6.2.1.7), oxalate--CoA ligase (EC 6.2.1.8), malate--CoA ligase (EC 6.2.1.9), acid--CoA ligase (GDP-forming) (EC 6.2.1.10), biotin--CoA ligase (EC 6.2.1.11), 4-coumarate--CoA ligase (EC 6.2.1.12), acetate--CoA ligase (ADP-forming) (EC 6.2.1.13), 6-carboxyhexanoate--CoA ligase (EC 6.2.1.14), arachidonate--CoA ligase (EC 6.2.1.15), acetoacetate--CoA ligase (EC 6.2.1.16), propionate--CoA ligase (EC 6.2.1.17), citrate--CoA ligase (EC 6.2.1.18), long-chain-fatty-a cid--luciferin-component ligase (EC 6.2.1.19), long-chain-fatty-acid--acyl-carrier protein ligase (EC 6.2.1.20), [citrate (pro-3S)-lyase] ligase (EC 6.2.1.22), dicarboxylate--CoA ligase (EC 6.2.1.23), phytanate--CoA ligase (EC 6.2.1.24), benzoate--CoA ligase (EC 6.2.1.25), O-succinylbenzoate--CoA ligase (EC 6.2.1.26), 4-hydroxybenzoate--CoA ligase (EC 6.2.1.27), 3-alpha,7-alpha-dihydroxy-5-beta-cholestanate--CoA ligase (EC 6.2.1.28), 3-alpha,7-alpha, 12-alpha-trihydroxy-5-beta-cholestanate--CoA ligase (EC 6.2.1.29), phenylacetate--CoA ligase (EC 6.2.1.30), 2-furoate--CoA ligase (EC 6.2.1.31), anthranilate--CoA ligase (EC 6.2.1.32), 4-chlorobenzoate-CoA ligase (EC 6.2.1.33), and trans-feruloyl-CoA synthase (EC 6.2.1.34). Methods of isolation and/or determining binding to and/or measuring activity of an acyl coenzyme A ligase are described in Aas and Bremer, 1968, Biochim Biophys Acta 164(2):157-66; Barth et al., 1971, Biochim Biophys Acta 248(1):24-33; Groot, 1975, Biochim Biophys Acta 380(1):12-20; Scholte et al., 1971, Biochim Biophys Acta 231(3):479-86; Scholte and Groot, 1975, Biochim Biophys Acta 409(3):283-96; Scaife and Tichivangana, 1980, Biochim Biophys Acta. 619(2):445-50; Man and Brosnan, 1984, Int J Biochem. 1984;16(12):1341-3; Patel and Walt, 1987, J Biol Chem. 262(15):7132-4; Philipp and Parsons, 1979, J Biol Chem. 254(21):19785-90; Vanden Heuvel et al., 1991, Biochem Pharmacol. 42(2):295-302; Youssefet al., 1994, Toxicol Lett. 74(1):15-21; and Vessey et al., 1999, Biochim Biophys Actal428(2-3):455-62. In certain specific embodiments, the fatty acid ligases are short chain fatty acid ligases. In such embodiments, preferred acyl coenzyme A mimics preferentially bind to or inhibit the activity of a short chain fatty acid ligase relative to a long chain fatty acid ligase.

Preferential binding by the acyl coenzyme A mimic to a short chain fatty acid ligase relative to a long chain fatty acid ligase means that the acyl coenzyme A mimic binds to the short chain fatty acid ligase with at least a 3-fold greater affinity more preferably with at least a 5-fold greater affinity, and most preferably with at least a 10-fold greater affinity than to the long chain fatty acid ligase. Preferential inhibition of a short chain fatty acid ligase relative to a long chain fatty acid ligase by the acyl coenzyme A mimic means that a particular amount or concentration of the acyl coenzyme A mimic inhibits the activity of the short chain fatty acid ligase by a degree of at least 50% more, more preferably at least 70% more, and yet more preferably at least 90% more than it inhibits the activity of the long chain fatty acid ligase. Thus, if an acyl coenzyme A mimic inhibits the activity of a a long chain fatty acid ligase by 40% at a given concentration, then the acyl coenzyme A mimic is said to inhibit the activity of the short chain fatty acid ligase by a degree of at least 50% more than it inhibits the activity of the long chain fatty acid ligase if it does so by 60% (40%+(50%×40%)).

As used herein, a short chain fatty acid ligase is an enzyme that catalyzes the addition of coenzyme A to an acyl coenzyme A molecule in which the acyl group comprises less than eight to ten carbon atoms. Further, as used herein, a long chain fatty acid ligase is an enzyme that catalyzes the addition of coenzyme A to an acyl coenzyme A molecule in which the acyl group comprises greater than twelve to sixteen carbon atoms.

In one embodiment, a biological sample known or suspected to have fatty acid ligase activity, most preferably short chain and long chain fatty acid ligase activity, is contacted with the test compound and the output of the ligase activity (i.e., measurement of acyl coenzyme A synthesis) or binding to the ligase by the test compound is measured. In one embodiment, the biological sample is a liver extract, for example a beef liver extract (see Mahler et al., 1953, J. BioL Chem. 204:453-468), or an adipose tissue extract. In another embodiment, the biological sample is a mitochondrial extract, a cytosol extract, a smooth endoplasmic reticulum extract, a microsomal extract, or a peroxisomal extract.

In other embodiments, the acyl coenzyme A metabolizing or binding proteins are enzymes or proteins involved in reactions utilizing acyl carrier protein (ACP). Exemplary ACPs include, but are not limited to, [acyl-carrier-protein] acetyltransferase (EC 2.3.1.38), [acyl-carrier-protein] malonyltransferase (EC 2.3.1.39), [acyl-carrier-protein] phosphodiesterase (EC 3.1.4.14); enoyl-[acyl-carrier-protein] reductase (NADPH) (EC 1.3.1.10), holo-[acyl-carrier-protein] synthase (EC 2.7.8.7), 3-oxoacyl-enzyme [acyl-carrier protein], 3-oxoacyl-[acyl-carrier-protein] reductase (EC 1.1.1.100 ), or 3-oxoacyl-[acyl-carrier-protein] synthase (EC 2.3.1.41).

In yet other embodiments, the acyl coenzyme A metabolizing or binding proteins are enzymes or proteins involved in reactions using Coenzyme A. Exemplary enzymes or proteins involved in reactions using Coenzyme A include, but are not limited to, acetate-coA ligase (EC 6.2.1.1), acetoacetyl-coA hydrolase (EC 3.1.2.11), acetoacetyl-coA: acetate coA transferase (EC 2.8.3.8 ), acetyl-coA acetyltransferase [thiolase] (EC 2.3.1.9), acetyl-coA acyltransferase (EC 2.3.1.16), acetyl-coA carboxylase (EC 6.4.1.2), [acetyl-coA carboxylase] phosphatase (EC 3.1.3.4), acetyl-coA ligase (EC 6.2.1.1), acyl-coA acyltransferase (EC 2.3.1.16), acyl-coA dehydrogenase (EC 1.3.99.3), acyl-coA dehydrogenase (NADP+) (EC 1.3.1.8), butyryl-coA dehydrogenase (EC 1.3.99.2), cholate-coA ligase (EC 6.2.1.7), dephospho-coA kinase (EC 2.7.1.24), enoyl-coA hydratase (EC 4.2.1.17), formyl-coA hydrolase (EC 3.1.2.10), glucan-1,4-α-glucosidase [glucoAmylase] (EC 3.2.1.3), glutaryl-coA dehydrogenase (EC 1.3.99.7), glutaryl-coA ligase (EC 6.2.1.6), 3-hydroxyacyl-coA dehydrogenase (EC 1.1.1.35), 3-hydroxybutyryl-coA dehydratase (EC 4.2.1.55), 3-hydroxybutyryl-coA dehydrogenase (EC 1.1.1.157), 3-hydroxyisobutyryl-coA hydrolase (EC 3.1.2.4), hydroxymethylglutaryl-coA lyase (EC 4.1.3.4), hydroxymethylglutaryl-coA reductase (EC 1.1.1.88), hydroxymethylglutaryl-coA reductase (NADPH) (EC 1.1.1.34), [hydroxymethylglutaryl-coA reductase (NADPH)] kinase (EC 2.7.1.109), [hydroxymethylglutaryl-coA reductase (nadph)] phosphatase (EC 3.1.3.47), hydroxymethylglutaryl-coA synthase (EC 4.1.3.5), lactoyl-coA dehydratase (EC 4.2.1.54), malonate-coA transferase (EC 2.8.3.3), malonyl-coA decarboxylase (EC 4.1.1.9), methylcrotonyl-coA carboxylase (EC 6.4.1.4), methylglutaconyl-coA hydratase (EC 4.2.1.18), methylmalonyl-coA carboxyltransferase (EC 2.1.3.1), methylmalonyl-coA decarboxylase (EC 4.1.1.41), methylmalonyl-coA epimerase (EC 5.1.99.1), methylmalonyl-coA mutase (EC 5.4.99.2), oxalate-coA transferase (EC 2.8.3.2), oxalyl-coA decarboxylase (EC 4.1.1.8), 3-oxoacid-coA transferase (EC 2.8.3.5), 3-oxoadipate coA-transferase (EC 2.8.3.6), palmitoyl-coA-enzyme palmitoyltransferase, propionate-coA ligase (EC 6.2.1.17), propionyl-coA carboxylase (EC 6.4.1.3), succinate-coA ligase (ADP-forming) (EC 6.2.1.5), succinate-coA ligase (GDP-forming) (EC 6.2.1.4), or succinate-propionate coA transferase.

In yet other embodiments, the acyl coenzyme A metabolizing or binding proteins are enzymes or proteins involved in reactions resulting in the biosynthesis or degradation of coA. Exemplary enzymes or proteins involved in reactions resulting in the biosynthesis or degradation of coA include, but are not limited to, pantothenatekinase (EC 2.7.1.33), pantothenate-B-alanine ligase (EC 6.3.2.1), phosphopantothenate-cysteine ligase (EC 6.3.2.5), pantetheine kinase (EC 2.7.1.34), pantetheine-phosphate adenylyltransferase (EC 2.7.7.3), 2-dehydropantoate reductase (EC 1.1.1.169), pantothenase (EC 3.5.1.22), pantothenoylcysteine decarboxylase (EC 4.1.1.30), phosphopantothenate-cysteine ligase (EC 6.3.2.5), phosphopantothenoylcysteine decarboxylase (EC 4.1.1.36).

In yet other embodiments, the acyl coenzyme A metabolizing or binding proteins are enzymes or proteins involved in the “mevalonate shunt,” as described in Edmond and Popjak, 1974, J. Biol. Chem. 249:66-71.

The present invention will be further understood by reference to the following non-limiting examples. The following examples are provided for illustrative purposes only and are not to be construed as limiting the invention scope of the invention in any manner.

### 6. EXPERIMENTAL

### 6.1. Examples

### Example 1

Synthesis of 2,4-Dihydroxy-N-[2-(4-hydroxy-3,3-dimethylbutylcarbamoyl)-ethyl]-3,3-dimethylbutyramide(S)

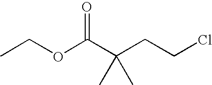

Ethyl 4-chloro-2,2-dimethylbutyrate. Under Ar-atmosphere, to a solution of ethyl isobutyrate (50.0 g, 0.43 mol) in anhydrous THF (300 mL) was added dropwise a solution of lithium diisopropylamide (2.0 M in heptane/THF/ethylbenzene, 237 mL, 0.47 mmol) over 50 min at −78° C. After stirring for 1.5 h at this temperature, 1-bromo-2-chloroethane (61.7 g, 0.43 mmol, 35.6 mL) was added dropwise over 30 min and the mixture was warmed to room temperature over 1 h. After 1 h at room temperature, the solution was poured into saturated NH₄Cl solution (1 L) and extracted with ethyl acetate (3×200 mL). The combined organic layers were washed with saturated NH₄Cl solution (200 mL) and saturated NaCl solution (200 mL), dried over MgSO₄, concentrated in vacuo, and dried in high vacuo. The residue (79.0 g) was purified by Kugelrohr distillation (85-90° C. air-bath temperature at 2 mm Hg) to afford ethyl 4-chloro-2,2-dimethylbutyrate (55.20 g, 72%) as a clear, colorless oil. Bp 85-90° C./2 mmHg (Kugelrohr) (lit. bp 54-56° C./0.25 mmHg, according to Kuwahara, M.; Kawano, Y.; Kajino, M.; Ashida, Y.; Miyake, A. Chem. Pharm. Bull. 1997, 45(9), 1447-1457). ¹H NMR (300 MHz, CDCl₃/TMS): δ (ppm): 4.14 (q, 2 H, J=7.1 Hz), 3.50 (m, 2 H), 2.25 (m, 2 H), 1.26 (t, 3 H, J=7.1 Hz), 1.22 (s, 6 H). ¹³C NMR (75 MHz, CDCl₃/TMS): δ (ppm): 176.87, 60.76, 43.25, 41.78, 40.85, 25.28, 14.25.

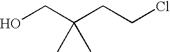

4-Chloro-2,2-dimethylbutan-1-ol. Under Ar-atmosphere, dichloromethane (150 mL) was added to lithium borohydride (9.2 g, 0.42 mmol) followed by dropwise addition of anhydrous methanol (13.6 g, 17.2 mL, 0.42 mmol) over 1 h at room temperature. After the H₂ effervescence had ceased, ethyl 4-chloro-2,2-dimethylbutyrate (50.5 g, 0.28 mmol) was added dropwise over 1 h. The reaction mixture was heated to reflux for 16 h, cooled to room temperature, and carefully hydrolyzed with saturated NH₄Cl solution (250 mL). The formed suspension was extracted with dichloromethane (3×100 mL). The combined organic layers were washed with 1 N HCl (200 mL) and saturated NaCl solution (100 mL), dried over MgSO₄, concentrated in vacuo, and dried in high vacuo to furnish 4-chloro-2,2-dimethylbutan-1-ol (36.6 g, 96%) as a clear, colorless oil. ¹H NMR (300 MHz, CDCl₃/TMS): δ (ppm): 3.57 (m, 2 H), 3.54-3.38 (m br., 1 H), 3.34 (s, 2 H), 1.80 (m, 2 H), 0.92 (s, 6 H). ¹³C NMR (75 MHz, CDCl₃/TMS): δ (ppm): 71.43, 41.98, 41.78, 35.66, 24.00.

2-(4-Chloro-2,2-dimethylbutyloxy)-tetrahydropyran. Under Ar-atmosphere, to a solution of 4-chloro-2,2-dimethylbutan-1-ol (35.3 g, 0.26 mmol) and p-toluenesulfonic acid monohydrate (260 mg, 1.4 mmol) in dichloromethane (200 mL) was added dropwise 3,4-dihydro-2H-pyran (27.2 g, 29.5 mL, 0.32 mmol) over 15 min at 0° C. After the addition, the reaction mixture was stirred at room temperature for 1 h, then filtered through a bed of aluminum oxide (activated, basic), concentrated in vacuo, and dried in high vacuo to afford 2-(4-chloro-2,2-dimethylbutyloxy)-tetrahydropyran (56.3 g, 98%) as a clear, colorless oil. A sample of 16.5 g was distilled in high vacuo to give the product (13.6 g) as a clear, colorless oil. Bp 75-84° C./0.5 mmHg. ¹H NMR (300 MHz, CDCl₃/TMS): δ (ppm): 4.55 (t, 1 H, J=2.9 Hz), 3.81 (m, 1 H), 3.57 (m, 1 H), 3.50 (m, 1 H), 3.48 (d, 1 H, J=9.3 Hz), 300 (d, 1H, J=9.3 Hz), 1.84 (m, 2 H), 1.80-1.46 (m, 6 H), 0.95 (s, 3 H), 0.94 (s, 3 H). ¹³C NMR (75 MHz, CDCl₃/TMS): δ (ppm): 98.08, 76.30, 62.02, 43.01, 41.59, 34.84, 30.67, 25.62, 24.72, 19.45. HRMS (LSIMS, nba): Calcd for C₁₁H₃₂ClO₂ (MH⁺): 221.1308, found: 221.1346.

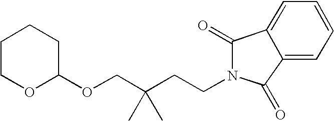

2-[3,3-Dimethyl-4-(tetrahydropyran-2-yloxy)-butyl]-isoindole-1,3-dione. To solution of 2-(4-chloro-2,2-dimethylbutyloxy)-tetrahydropyran (1.10 g, 5 mmol) in anhydrous DMF (10 mL) was added potassium phthalimide (0.93 g, 5 mmol) at room temperature. The reaction mixture was heated to 90° C. for 6 h. After cooling, the reaction mixture was poured into ice-water (100 mL). The product was extracted with ethyl acetate (3×30 mL). The combined organic layers were dried over sodium sulfate and concentrated in vacuo to give the crude product (1.36 g), which was purified by column chromatography (silica gel, hexanes:ethyl acetate=4:1) to furnish the desired product (0.92 g, 55.8%) as a colorless oil. ¹H NMR (300 MHz, CDCl₃/TMS): δ (ppm): 7.90-7.80 (m, 2 H), 7.80-7.60 (m, 2 H), 4.59 (t, J=3.2 Hz, 1 H), 3.85 (m, 1 H), 3.75 (t, J=8.3 Hz, 2 H), 3.53 (d, J=9.1 Hz, 1 H), 3.50 (m, 1 H), 3.07 (d, J=9.1 Hz, 1 H), 1.90-1.40 (m, 8 H), 1.03 (s, 3 H), 1.01 (s, 3 H). ¹³C NMR (75 MHz, CDCl₃/TMS): δ (ppm): 168.2, 133.7, 132.2, 123.0, 99.0, 76.3, 61.8, 37.6, 34.4, 33.8, 30.5, 25.5, 24.6, 24.4, 19.3. HRMS (LSIMS, nba): Calcd for C₁₉H₂₆NO₄(MH⁺): 332.1861; found: 332.1860.

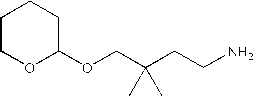

3,3-Dimethyl-4-(tetrahydropyran-2-yloxy)-butylamine. A solution of 2-[3,3-dimethyl-4-(tetrahydropyran-2-yloxy)-butyl]-isoindole-1,3-dione (0.662 g, 2 mmol) in absolute ethanol (4 mL) was heated to 70° C. for 10 min until the starting material was completely dissolved. Hydrazine monohydrate (85%, 0.2 g, 3.4 mmol) was added and the reaction mixture was heated to reflux for 1 h. The formed solid was removed by filtration. The filtrate was concentrated in vacuo. The crude product was dissolved in chloroform (60 mL) and washed with 10% sodium bicarbonate solution. The organic layer was separated and the aqueous solution was extracted with chloroform (2×30 mL). The combined organic layers were dried over sodium sulfate and concentrated in vacuo to give the pure product (0.32 g, 80%) as a light yellow oil. ¹H NMR (300 MHz, CDCl₃/TMS): δ (ppm): 4.55 (t, J=3.0 Hz, 1 H), 3.90-3.70 (m, 1 H), 3.50 (m, 1 H), 3.47 (d, J=9.0 Hz, 1 H), 2.98 (d, J=9.0 Hz, 1 H), 2.72 (pseudo-t, 2 H), 1.95-1.35 (m, 8 H), 1.23 (br., 2 H), 0.92 (s, 3 H), 0.91 (s, 3 H). ¹³C NMR (75 MHz, CDCl₃/TMS): δ (ppm): 99.0, 76.6, 61.9, 43.7, 37.8, 33.8, 30.6, 25.5, 24.7, 19.4. HRMS (LSIMS, nba): Calcd for C₁₁H₂₄NO₂ (MH+): 202.1807; found: 202.1806.

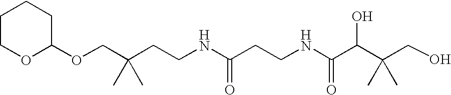

N-{2-[3,3-Dimethyl-4-(tetrahydropyran-2-yloxy)-butylcarbamoyl]-ethyl}-2,4-dihydroxy-3,3-dimethylbutyramide. D-pantothenic acid, sodium salt (1.8 g, 7.5 mmol) was dissolved in a DMF/dichloromethane mixture (40 mL/26 mL). To the above solution was added N-hydroxysuccinimide (0.87 g, 7.5 mmol), followed by N,N′-dicyclohexyl-carbodiimide (DCC) (1.67 g, 8.1 mmol). The reaction was kept at room temperature for 3 h. 3,3-Dimethyl-4-(tetrahydropyran-2-yloxy)-butylamine (1.33 g, 6.62 mmol) in a DMF/dichloromethane mixture (3 mL/2 mL) was added and stirring was continued for 13 h. The reaction mixture was filtered and the filtrate was concentrated in high vacuum to obtain the crude product (3.6 g). Purification by flash chromatography on silica gel (first: ethyl acetate; second: ethyl acetate:hexanes=4:1) afforded the desired compound as a colorless oil (1.43 g, 53.8%). ¹H NMR (300 MHz, CD₃CN/TMS): δ (ppm): 7.65 (br., 1 H), 7.28 (br., 1 H), 4.98 (d, J=5.2 Hz, 1 H), 4.53 (s, 1 H), 4.32 (m, 1 H), 3.92 (d, J=4.9 Hz, 1 H), 3.84-3.70 (m, 1 H), 3.50-3.28 (m, 6 H), 3.28-3.12 (m, 2 H), 2.99 (d, J=9.0 Hz, 1 H), 2.38 (t, J=6.3 Hz, 2 H), 1.86-1.70 (m, 1 H), 1.70-1.36 (m, 7 H), 0.93 (s, 3 H), 0.91 (s, 6 H), 0.86 (s, 3 H). ¹³C NMR (75 MHz, CD₃CN/TMS): δ (ppm): 174.6, 171.9, 100.0, 77.8, 77.1, 71.1, 62.6, 40.5, 39.9, 36.7, 36.4, 34.7, 31.8, 26.7, 25.4, 22.3, 21.1, 20.6. HRMS (ESI): Calcd for C₂₀H₃₈N₂O₆Na (MNa⁺): 425.2622; found: 425.2652.

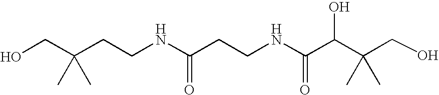

2,4-Dihydroxy-N-[2-(4-hydroxy-3,3-dimethylbutylcarbamoyl)-ethyl]-3,3-dimethylbutyramide. A solution of N-{2-[3,3-dimethyl-4-(tetrahydropyran-2-yloxy)-butylcarbamoyl]-ethyl}-2,4-dihydroxy-3,3-dimethylbutyramide (1.44 g, 3.58 mmol) and pyridinium p-toluenesulfonate (0.18 g, 0.72 mmol) in absolute ethanol (32 mL) was stirred at 55° C. for 6 h. The reaction mixture was evaporated to dryness. The residue (1.2 g) was dissolved in methanol (10 mL) and sodium carbonate solution (0.5 g in 10 mL of water) was added. The solution was evaporated to dryness. The residue was purified by column chromatography (silica gel, chloroform:ethanol=4:1, R_{f}=0.4) to obtain the product as a foam (0.8 g, 63.5%). ¹H NMR (300 MHz, CD₃OD/TMS): δ (ppm): 4.11 (s, 1 H), 3.80-3.50 (m, 4 H), 3.45 (s, 2 H), 3.45-3.25 (m, 2 H), 2.65-2.50 (m, 2 H), 1.65 (t, J=8.5 Hz, 2 H), 1.12 (s, 6 H), 1.09 (s, 6 H). ¹³C NMR (75 MHz, CD₃OD/TMS): δ (ppm): 175.9, 173.3, 77.2 71.7, 70.3, 40.4, 38.8, 36.6, 35.5, 24.6, 21.4, 21.2. HRMS (LSIMS, gly): Calcd for C₁₅H₃₁N₂O₅ (MH⁺): 319.2232, found: 319.2217.

### Example 2

N-[3-(2,4-Dihydroxy-3,3-dimethylbutyrylamino)-2-hydroxypropyl]-2,4-dihydroxy-3,3-dimethyl-butyramide (W)

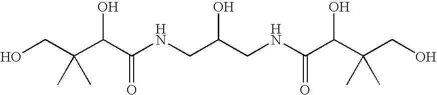

To a solution of pantolactone (5.2 g, 40 mmol) in absolute ethanol (50 mL) was added 1,3-diamino-isopropanol (1.8 g, 20 mmol). The reaction mixture was heated to reflux for 72 h and concentrated. The residue was purified by column chromatography (silica gel, ethyl acetate, R_{f}=0.5) to obtain a foamy solid (6 g). Recrystallization from methanol gave a white solid (1.6 g, mp 169-171 ° C.). The mother liquor was purified by chromatography (silica, ethyl acetate) to obtain another portion of the product (2.6 g), giving a combined yield of 60%. Mp 169-171 ° C. (methanol). ¹H NMR (300 MHz, CD₃OD/TMS): δ (ppm): 4.90 (br., 7 H), 3.92 (s, 2 H), 3.80-3.65 (m, 1 H), 3.46 (d, J=11.0Hz, 2 H), 3.40 (d, J=11.0 Hz, 2 H), 3.30-3.18 (m, 4 H), 0.94 (s, 12H). ¹³C NMR (75 MHz, CD₃OD/TMS): δ (ppm): 176.7, 77.5, 70.4, 43.3, 40.6, 21.6, 21.1. HRMS (LSIMS, gly): Calcd for C₁₅H₃₁N₂O₇ (MH⁺): 351.2131, found: 351.2136. HPLC (Alltima C₈, 5μ, 4.6 mm×250 mm, acetonitrile/0.05 M aqueous KH₂PO₄=70/30, flow rate 1 mL/min, RI detection, retention time 2.55 min): 98.9%.

### Example 3

N-[2-(2,4-Dihydroxy-3,3-dimethylbutyrylamino)-ethyl]-2,4-dihydroxy-3,3-dimethylbutyramide (racemic) (V2)

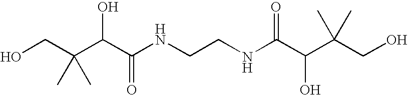

Under argon atmosphere, a solution of pantolactone (5.0 g, 38 mmol) and ethylenediamine (1.2 g, 19 mmol) in ethanol (50 mL) was heated to reflux for two days. The reaction mixture was concentrated to dryness and redissolved in ethanol (100 mL). This solution was passed through an Amberlyst-15 ion-exchange column (strongly acidic, pre-washed with HCl, deionized water, and ethanol), eluting with additional ethanol (900 mL). Concentration and vacuum drying afforded the crude product (5.24 g, 86% yield) as a clear, colorless glass. Recrystallization from hexanes/ethyl acetate gave the product as a waxy material (1.18 g, 25% recovery). ¹H NMR (300 MHz, CD₃OD/TMS): δ (ppm): 3.90 (s, 2 H), 3.50-3.37 (m, 4 H), 3.35 (s, 4 H), 0.93 (s, 12 H). ¹³C NMR (75 MHz, CD₃OD/TMS): δ (ppm): 176.6, 77.5, 70.4, 40.5, 39.8, 21.5, 21.1. Anal. Calcd. for C₁₄H₂₈N₂O₆: C, 52.48; H, 8.81; N, 8.74. Found: C, 52.35; H, 8.81; N, 8.54.

### Example 4

(R,S)-N-[2-(2,4-Dihydroxy-3,3-dimethylbutyrylamino)-ethyl]-2,4-dihydroxy-3,3-dimethyl-butyramide (meso compound) (V1).

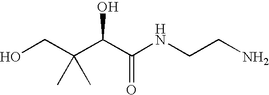

(R)-N-(2-Aminoethyl)-2,4-dihydroxy-3,3-dimethylbutyramide. A solution of (R)-(−)-pantolactone (22.4 g, 172 mmol) and ethylenediamine (21.5 g, 358 mmol) in ethanol (100 mL) was heated to reflux for three days. The solution was concentrated in vacuo and the residue (42.28 g) was purified by column chromatography (short silica column, 20% ethanol/dichloromethane). The purified material (36.32 g) was recrystallized twice from methyl tert.-butyl ether/ethanol, affording the compound as white plates (21.26 g, 62% yield). [α]_{D}=+67.6 (c=1.06, 25° C., methanol). ¹H NMR (300 MHz, CDCl₃/TMS): δ (ppm): 3.90(s, 1 H), 3.47 (d, 1 H, J=11.0 Hz),3.39(d, 1 H, J=11.0 Hz), 3.29(t, 2 H, J=6.0Hz), 2.74 (t, 2 H, J=6.0 Hz), 0.93 (s, 6 H). ¹³C NMR (75 MHz, CDCl₃/TMS): δ (ppm): 176.6, 77.6, 70.4, 42.5, 42.1, 40.4, 21.6, 21.1.

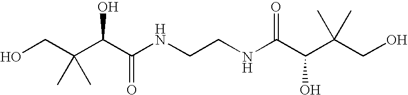

(R,S-N-[2-(2,4-Dihydroxy-3,3-dimethylbutyrylamino)-ethyl]-2,4-dihydroxy-3,3-dimethyl-butyramide (meso compound). A solution of (2R)-N-(2-aminoethyl)-2,4-dihydroxy-3,3-dimethylbutyramide (15.4 g, 76.9 mmol) and (S)-(+)-pantolactone (10.0 g, 76.1 mmol) in ethanol (120 mL) was heated to reflux for three days. The solvent was removed under reduced pressure. The residue was dissolved in methyl tert.-butyl ether (550 mL) and ethanol (50 mL) and kept at −5° C. overnight. A viscous oil or wax separated and the supernatant liquid was decanted. The residue was dried and dissolved in ethyl acetate (100 mL) and ethanol (5 mL), stored at −5° C. for three days, and the supernatant was again decanted. The residue was dried in high vacuo to afford the product as a viscous oil (8.40 g, 33% yield). [α]_{D}=−0.76 (c=1.19, 24 ° C., methanol). ¹H NMR (300 MHz, CDCl₃/TMS): δ (ppm): 3.82 (s, 1 H), 3.38 (d, 1 H, J=5.5 Hz), 3.30 (d, 1 H, J=5.5 Hz), 3.27 (s, 2 H), 0.83 (s, 6 H). ¹³C NMR (75 MHz, CDCl₃/TMS): δ (ppm): 176.5, 77.3, 70.4, 40.4, 39.7, 21.5, 21.1.

### Example 5

(R,R)-N-[2-(2,4-Dihydroxy-3,3-dimethylbutyrylamino)-ethyl]-2,4-dihydroxy-3,3-dimethylbutyramide (V3)

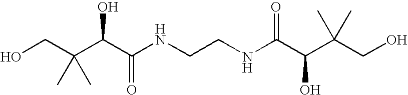

A solution of (R)-pantolactone (5.0 g, 38 mmol) and ethylenediamine (1.1 g, 18 mmol) was heated to reflux in ethanol (25 mL) for three days. Evaporation of the solvent gave the crude material (5.87 g), which was recrystallized from hot ethyl acetate (100 mL) containing just enough ethanol to fully dissolve the product. Upon cooling, sharp, rock-salt like crystals appeared, which were filtered and dried to afford the (R,R) product (3.39 g, 56% yield). M.p.: 124.8-124.9° C. [α]_{D}=+67.6 (c=1.06, 25° C., methanol). ¹H NMR (300 MHz, DMSO-d₆/TMS): δ (ppm): 7.84 (s, 2 H), 5.35 (d, 2 H, J=5.0 Hz), 4.49 (m, 2 H), 3.71 (d, 2 H, J=5.0 Hz), 3.50-3.14 (m, 8 H), 0.81 (s, 6 H), 0.79 (s, 6 H). ¹³C NMR (75 MHz, DMSO-d₆/TMS): δ (ppm): 173.3, 75.1, 68.0, 39.0, 38.3, 21.1, 20.4. HRMS (LSIMS, gly): Calcd. for C₁₄H₂₉N₂O₆ (MH⁺): 321.2026, found: 321.2034. HPLC: 97.5% purity. Anal. Calcd. for C₁₄H₂₈N₂O₆: C, 52.48; H, 8.81; N, 8.74. Found: C, 52.05; H, 8.82; N, 8.79.

### Example 6

(S,S)-N-8 2-(2,4-Dihydroxy-3,3-dimethylbutyrylamino)-ethyl]-2,4-dihydroxy-3,3-dimethylbutyramide (V4)

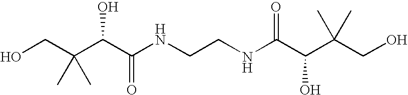

A solution of(S)-pantolactone (5.0 g, 38 mmol) and ethylenediamine (1.1 g, 18 mmol) was heated to reflux in ethanol (25 mL) for three days. The solution was concentrated in vacuo to give the crude material (6.18 g), which was recrystallized from hot ethyl acetate (100 mL) containing just enough ethanol to fully dissolve the product. Sharp, rock-salt like crystals were obtained, which were filtered and dried, affording the (S,S) product (4.25 g, 70% yield). M.p.: 124.8-124.9° C. [α]_{D}=−69.2 (c=1.09, 25° C., methanol). ¹H NMR (300 MHz, DMSO-_{d}/TMS): δ (ppm): 7.84 (s, 2 H), 5.35 (d, 2 H, J=5.0 Hz), 4.49 (m, 2 H), 3.71 (d, 2 H, J=5.0 Hz), 3.50-3.14 (m, 8 H), 0.81 (s, 6 H), 0.79 (s, 6 H). ¹³C NMR (75 MHz, DMSO-d₆/TMS): δ (ppm): 173.3, 75.1, 68.0, 39.0, 38.3, 21.1, 20.4. HRMS (LSIMS, gly): Calcd. for C₁₄H₂₉N₂O₆ (MH⁺): 321.2026, found: 321.2041. HPLC: 99.2% purity. Anal. Calcd. for C₁₄H₂₈N₂O₆: C, 52.48; H, 8.81; N, 8.74. Found: C, 52.29; H, 8.82; N, 8.81; N, 8.82, N, 8.82.

### Example 7

N-{2-[2-(2,4-Dihydroxy-3,3-dimethylbutyrylamino)-ethoxy]-ethyl}-2,4-dihydroxy-3,3-dimethylbutyramide (U)

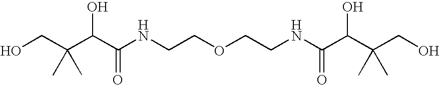

A mixture of pantolactone (7.25 g, 55.1 mmol), 2,2′-oxy-bis(ethylamine) dihydrochloride (5.03 g, 27.6 mmol) and sodium bicarbonate (4.78 g, 56.9 mmol) in ethanol (100 mL) was heated to reflux under an argon atmosphere for three days. After cooling to room temperature, the solids were filtered and the filtrate was evaporated to dryness. The crude material (12.20 g) was purified by flash chromatography on silica (0-40% ethanol/chloroform) to give the target compound as a clear, colorless oil (7.92 g, 79% yield). ¹H NMR (300 MHz, CD₃OD/TMS): δ (ppm): 3.91 (s, 2 H), 3.6-3.3 (m, 14 H), 0.93 (s, 12 H). ¹³C NMR (75 MHz, CD₃OD/TMS): δ (ppm): 176.2, 77.4, 70.5, 70.4, 40.5, 39.8, 21.5, 21.0.

### Example 8

Synthesis of 2,4-dihydroxy-N-{4-[4-(2,3,4-tri-O-acetyl-α-D-xylopyranosyl)-butoxy]-2,6-dimethyl-phenyl}-3,3-dimethyl-butyramide (AG)

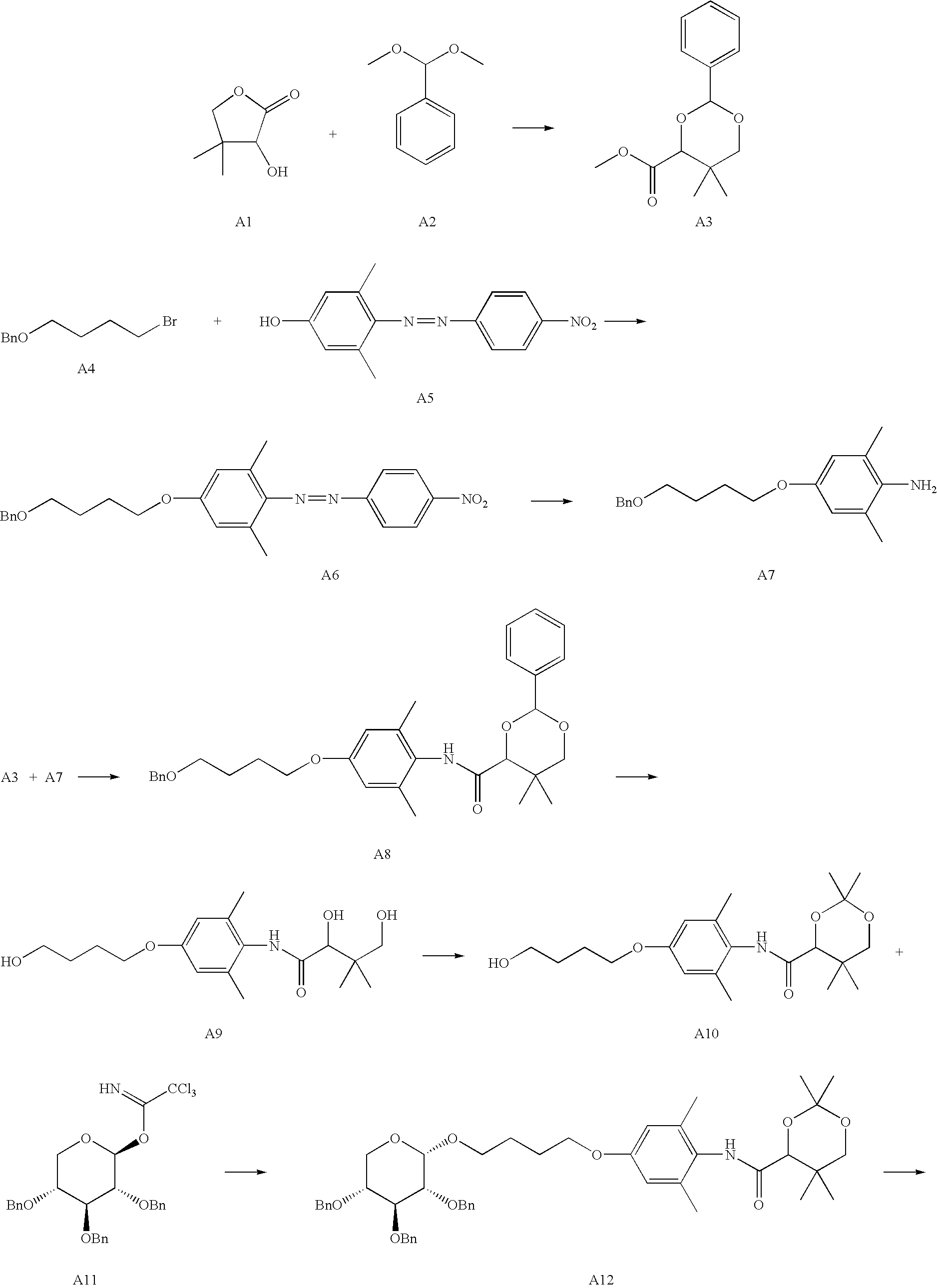

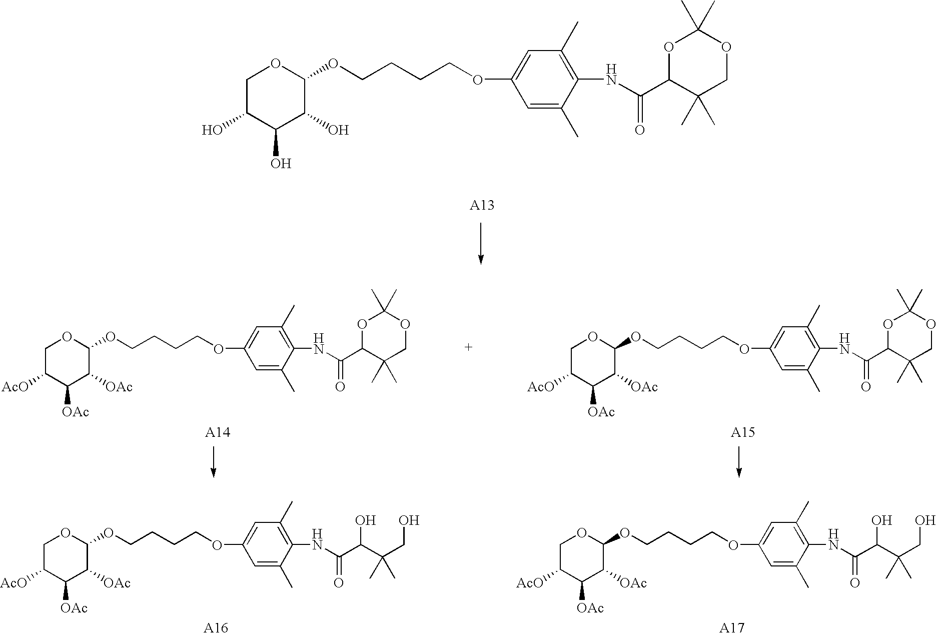

5,5-Dimethyl-2-phenyl-[1,3]-dioxane-4-carboxylic acid methyl ester. To a solution of (D,L)-pantolactone (20.4 g, 156 mmol) and benzaldehyde dimethylacetal (40 mL, 265 mmol) in 1,4-dioxane (100 mL) was added TsOH.(0.606 g, 3.2 mmol). The reaction mixture was stirred for 2 days, treated with NaHCO₃ (5.1 g), and stirred for another 3 h. Et₂O (250 mL) was added and the resulting mixture was washed successively with a mixture of saturated aq. NaHCO₃ solution (100 mL) and water (200 mL) and brine (100 mL), dried (Na₂SO₄), and concentrated in vacuo to give a liquid (52.2 g). Column chromatography (silica, heptane/EtOAc=7:1) of this liquid gave a white solid (15.5 g), which was recrystallized from heptane (30 mL) to give 5,5-dimethyl-2-phenyl-[1,3]-dioxane-4-carboxylic acid methyl ester (14.0 g, 36%, mp 86.5-88° C.) as colorless crystals. ¹H-NMR (CDCl₃) δ (ppm)=7.54-7.50 (m, 2H), 7.38-7.29 (m, 3H), 5.46 (s, 1H), 4.23 (s, 1H), 3.73 (s, 3H), 3.72 (d, J=11.4 Hz, 1H), 3.64 (d, J=11.4 Hz, 1H), 1.18 (s, 3H), 0.96 (s, 3H); ¹³C-NMR (CDCl₃) δ (ppm)=168.9, 137.5, 128.9, 128.1 (2×), 126.1 (2×), 101.4, 83.7, 78.1, 51.5, 32.7, 21.5, 19.4; Anal. calcd for C₁₄H₁₈O₄: C, 67.18; H, 7.25; found: C, 67.18; H, 7.23.

[4-(4-Benzyloxy-butoxy)-2,6-dimethyl-phenyl]-(4-nitro-phenyl)-diazene. To a solution of (4-bromobutoxymethyl)-benzene (18.3 g, 75.2 mmol, prepared according to: Comins, D. L.; LaMunyon, D. H.; Chen, X., J Org. Chem., 1997, 62, 8182-8187) and 3,5-dimethyl-4-(4-nitro-phenylazo)-phenol (19.59 g, 72.3 mmol, prepared according to: Smith, L. I.; Irwin, W. B., J Am. Chem. Soc., 1941, 63, 1036-1043) in DMSO (100 mL) was added K₂CO₃ (10.4 g, 75.2 mmol). The mixture was stirred overnight and then poured into a mixture of ice and water (300 mL). The amorphous solid material was filtered, washed with water (4×75 mL), and air dried. Purification of the residue (28.3 g) by column chromatography (silica, heptane:EtOAc=12:1) gave [4-(4-benzyloxy-butoxy)-2,6-dimethyl-phenyl]-(4-nitro-phenyl)-diazene (18.4 g, 63%) as a crystalline solid. An analytical sample of [4-(4-benzyloxy-butoxy)-2,6-dimethyl-phenyl]-(4-nitro-phenyl)-diazene (0.682 g, mp: 68-69.5° C., red brown needles) was obtained by recrystallization of 0.757 g from 2-propanol (50 mL). ¹H-NMR (CDCl₃) δ (ppm)=8.34 (d with fine splitting, J=9 Hz, 2H), 7.91 (d with fine splitting, J=9 Hz, 2H), 7.36-7.25 (m, 5H), 6.67 (s, 2H), 4.53 (s, 2H), 4.05 (t, J=6.2 Hz, 2H), 3.56 (t, J=6.0 Hz, 2H), 2.56 (s, 6H), 1.97-1.88 (m, 2H), 1.86-1.76 (m, 2H); ¹³C-NMR (CDCl₃) δ (ppm)=160.7, 156.6, 148,0, 143.7, 138.5, 137.2 (2×), 128.4 (2×), 127.62 (2×), 127.56, 124.7 (2×), 122.7 (2×), 115.3 (2×), 72.9, 69.8, 67.8, 26.3, 26.1, 21.1 (2×); Anal. calcd for C₂₅H₂₇N₃O₄: C, 69.27; H, 6.28; N, 9.69, found: C, 69.26; H, 6.17; N, 9.59.

4-(4-Benzyloxy-butoxy)-2,6-dimethyl-phenylamine. A mixture of [4-(4-benzyloxy-butoxy)-2,6-dimethyl-phenyl]-(4-nitro-phenyl)-diazene (18.35 g, 42.4 mmol) and sodium dithionite (73.7 g, 0.424 mol) in EtOH (460 mL) and water (460 mL) was stirred under reflux for 1.5 h. An almost colorless mixture was obtained, which was allowed to cool to rt, concentrated in vacuo to a volume of approximate 450 mL and then extracted wit Et₂O (1×300 mL, 2×100 mL). The combined organic layers were washed with brine (100 mL), dried (Na₂SO₄), and concentrated in vacuo to give an oil (12.7 g), which was purified by column chromatography (silica, heptane:EtOAc=4:1) to give 4-(4-benzyloxy-butoxy)-2,6-dimethyl-phenylamine (10.6 g, 84%) as a brown oil. ¹H-NMR (CDCl₃) δ (ppm)=7.33-7.26 (m, 5H), 6.54 (s, 2H), 4.50 (s, 2H), 3.88 (t, J=5.9 Hz, 2H), 3.52 (t, J=5.9 Hz, 2H), 3.17 (br s, 2H), 2.15 (s, 6H), 1.84-1.77 (m, 4H); ¹³C-NMR (CDCl₃) δ (ppm)=151.4, 138.6, 136.3, 128.3 (2×), 127.6 (2×), 127.4, 123.1 (2×), 114.7 (2×), 72.8, 70.0, 68.2, 26.35, 26.27, 17.9 (2×); HRMS calcd for C₁₉H₂₅NO₂ (M)⁺: 299.1885, found: 299.1881.

5,5-Dimethyl-2-phenyl-[1,3]dioxane-4-carboxylic acid [4-(4-benzyloxy-butoxy)-2,6-dimethyl-phenyl]-amide. A solution of 5,5-dimethyl-2-phenyl-[1,3]-dioxane-4-carboxylic acid methyl ester (9.87 g, 39.5 mmol) in MeOH (200 mL) was treated with LiOH.H₂O (1.99 g, 47.4 mmol) and water (6 mL). The reaction mixture was stirred for 2 days at 40° C., concentrated in vacuo, and coevaporated from toluene (2×100 mL). The remaining thin oil was dissolved in toluene (300 mL) and concentrated to an amount of ˜200 mL. The resultant solution was treated with SOCl₂ (4.0 mL, 6.5 g, 54 mmol) stirred at room temperature for 1 h, cooled to −40° C., and then treated with pyridine (40 mL). The cooling bath was removed and a solution of 4-(4-benzyloxy-butoxy)-2,6-dimethyl-phenylamine (10.62 g, 35.5 mmol) in pyridine (40 ml) was immediately added at once. The reaction mixture was stirred for 45 min and then poured into a mixture of water and ice (1 L). After 1 h, the obtained mixture was separated and the water layer was extracted with toluene (2×200 mL). The combined organic layers were successively washed with a mixture of aqueous HCl (4 M, 350 mL) and ice (150 mL), brine (150 mL), and a saturated aqueous solution of NaHCO₃ (150 mL), dried (Na₂SO₄), and concentrated in vacuo. The remaining oil (19.6 g) was purified by column chromatography (silica, heptane:EtOAc=3:2) to give an oil, which was coevaporated from Et₂O (100 mL) to give 5,5-dimethyl-2-phenyl-[1,3]dioxane-4-carboxylic acid [4-(4-benzyloxy-butoxy)-2,6-dimethyl-phenyl]-amide (13.9 g, 76%) as a dark yellow oil. ¹H-NMR (CDCl₃) δ (ppm)=7.70 (br s, 1H), 7.53-7.50 (m, 2H), 7.41-7.36 (m, 3H), 7.31-7.22 (m, 5H), 6.56 (s, 2H), 5.59 (s, 1H), 4.49 (s, 2H), 4.30 (s, 1H, 3.91 (t, J=6.0 Hz, 2H), 3.79 (d, J=11.3 Hz, 1H), 3.72 (d, J=11.3 Hz, 1H), 3.51 (t, J=6.0 Hz, 2H), 2.17 (s, 6H), 1.89-1.71 (m, 4H). 1.31 (s, 3H), 1.17 (s, 3H); ¹³C-NMR (CDCl₃) δ (ppm)=167.3, 157.4, 138.3, 137.6, 136.2 (2×), 129.0, 128.2 (2×), 128.1 (2×), 127.4 (2×), 127.3, 125.9 (2×), 125.7, 113.9 (2×), 101.4, 84.1 78.7, 72.9, 69.9, 67.7, 33.7, 26.5, 26.3, 22.1, 19.8, 19.1 (2×); HRMS calcd for C₃₂H₃₉NO₅ (M⁺): 517.2828, found: 517.2829.

2,4-Dihydroxy-N-[4-(4-hydroxy-butoxy)-2,6-dimethyl-phenyl]-3,3-dimethyl-butyramide. Under N₂ atmosphere, Pd on C (10% (w/w), 1.0 g, 0.94 mmol) was added to a solution of 5,5-dimethyl-2-phenyl-[1,3]dioxane-4-carboxylic acid [4-(4-benzyloxy-butoxy)-2,6-dimethyl-phenyl]-amide (13.5 g, 26.2 mmol) in EtOH (200 mL). The reaction vessel was flushed with H₂ gas and the reaction mixture was stirred under H₂ atmosphere at 5 bar for 24 h. TLC analysis indicated that no starting material was converted. Therefore, the reaction mixture was filtered and the residue was washed with EtOH (5×50 mL). The filtrate and washings were combined, concentrated in vacuo to a volume of ˜100 mL and then EtOH (200 mL) was added. The resulting solution was treated with Pd on C (10% (w/w), 1.0 g, 0.94 mmol) and hydrogenated at 5 bar for 24 h. TLC analysis of the reaction mixture indicated an incomplete reaction. Therefore, again the reaction mixture was filtered and the residue was washed with EtOH (5×50 mL). The filtrate and washings were combined, concentrated in vacuo to a volume of ˜100 mL and then EtOH (200 mL) was added. The resulting solution was treated with Pd on C (10% (w/w), 1.0 g, 0.94 mmol) and hydrogenated at 5 bar for 3 days, filtered and the residue was washed with EtOH (4×50 mL). The combined filtrate and washings were concentrated in vacuo and concentrated from toluene (2×100 mL) to give an oil, which was crystallized from a mixture of EtOAc and iPr₂O to give 2,4-dihydroxy-N-[4-(4-hydroxy-butoxy)-2,6-dimethyl-phenyl]-3,3-dimethyl-butyramide (10.8 g, 84%) as yellowish crystals. mp 101-103° C. ¹H-NMR (DMSO-d6) δ (ppm)=8.89 (s, 1H), 6.62 (s, 2H), 5.60 (d, J=5.9 Hz, 1H, exchanges on addition of D₂O), 4.52 (d, J=5.9 Hz, 1H, exchanges on addition of D₂O), 4.43 (d, J=5.2 Hz, 1H, exchanges on addition of D₂O), 3.93 (s, 1H), 3.93 (t, J=5.7 Hz, 2H), 3.48-3.37 (m, 3H), 3.26 (dd, J=10.4, 5.2 Hz, 1H), 2.11 (s, 6H), 1.76-1.67 (m, 2H). 1.59-1.50 (m, 2H), 0.94 (s, 3H), 0.93 (s, 3H); ¹³C-NMR (DMSO-d6) δ (ppm)=171.5, 156.2, 136.0 (2×), 127.6, 113.0 (2×), 75.4, 68.0, 67.2, 60.3, 39.2, 29.0, 25.5, 21.3, 20.5, 18.8 (2×); Anal. calcd for C₁₈H₂₉NO₅: C, 63.69; H, 8.61; N, 4.13, found: C, 63.96; H, 8.68; N, 3.85.

2,2,5,5-Tetramethyl-[1,3]dioxane-4-carboxylic acid [4-(4-hydroxy-butoxy)-2,6-dimethyl-phenyl]-amide. A mixture of 2,4-dihydroxy-N-[4-(4-hydroxy-butoxy)-2,6-dimethyl-phenyl]-3,3-dimethyl-butyramide (7.31 g, 21.6 mmol) in 2,2-dimethoxypropane (10 mL, 8.4 g, 81 mmol) and 1,4-dioxane (100 mL) was treated with p-TsOH.H₂O (200 mg, 1.05 mmol), stirred for 1.5 h, treated with NaHCO₃ (2.5 g), stirred for 1 h, and then set aside during the weekend. The mixture was filtered, and the filtrate was concentrated in vacuo to give a solid material, which was dissolved in EtOAc (100 mL) and then filtered through a layer of silicagel in a glassfilter. The residue was eluted with EtOAc (5×10 mL) and the filtrate and eluates were combined and concentrated in vacuo to a volume of ˜30 mL. Heptane was added to the resultant solution until spontaneous crystallization started. The obtained crystalline mass was filtered, washed with a mixture of heptane and EtOAc (10:1, 3×20 mL) and air dried to give 2,2,5,5-tetramethyl-[1,3]dioxane-4-carboxylic acid [4-(4-hydroxy-butoxy)-2,6-dimethyl-phenyl]-amide (5.45 g, 67%) as colorless crystals. The mother liquor was concentrated in vacuo to give an oil, which consisted mainly of more apolar products, which were not characterized, but dissolved in a mixture of HOAc and water (4:1, 10 mL) and stirred for 15 min. NaOAc (2.5 g) and water (20 mL) were added to the resultant solution, and after 15 min, the formed crystalline material was filtered, washed with water (3 ×10 mL), air dried, and recrystallized from 2-propanol/water to give another crop of 2,2,5,5-tetramethyl-[1,3]dioxane-4-carboxylic acid [4-(4-hydroxy-butoxy)-2,6-dimethyl-phenyl]-amide (2.09 g, 26%) as colorless crystals. ¹H-NMR (CDCl₃) δ (ppm)=7.77 (s, 1H), 6.62 (s, 2H), 4.29 (s, 1H), 3.97 (t, J=6.0 Hz, 2H), 3.76 (d, J=11.7 Hz, 1H), 3.70 (t, J=6.1 Hz, 2H), 3.35 (d, J=11.7 Hz, 1H) 2.20 (s, 6H), 1.90-1.82 (m, 2H). 1.78-1.69 (m, 2H), 1.52 (s, 3H), 1.50 (s, 3H), 1.19 (s, 3H), 1.11 (s, 3H); ¹³C-NMR (CDCl₃) δ (ppm)=168.3, 157.5, 136.4 (2×), 126.2, 114.0 (2×), 99.3, 77.6, 71.7, 67.8, 62.4, 33.3, 29.51, 29.46, 25.8, 22.1, 19.4, 18.9 (2×), 18.8; Anal. calcd for C₂₁H₃₃NO₅: C, 66.46; H, 8.76; N, 3.69, found: C, 66.42; H, 8.92; N, 3.65.

2,2,5,5-Tetramethyl-[1,3]dioxane-4-carboxylic acid {4-[4-(2,3,4-tri-O-benzyl-α-D-xylopyranosyl)-butoxyl-2,6-dimethyl-phenyl]-amide. To a mixture of O-( 2,3,4-tri-O-benzyl-β-D-xylopyranosyl)-trichloroacetimidate (13.6 g, 24.0 mmol, prepared according to: Schmidt, R. R.; Michel, J.; Roos, M., Liebigs Ann. Chem., 1984, 1343-1357), 2,2,5,5-tetramethyl-[1,3]dioxane-4-carboxylic acid [4-(4-hydroxy-butoxy)-2,6-dimethyl-phenyl]-amide (7.00 g, 18.5 mmol) in Et₂O (140 mL) and 1,2-dichloroethane (70 mL) was added trimethylsilyltriflate (0.30 mL, 0.26 g, 1.17 mmol), under a nitrogen atmosphere at −78° C. After 45 min at −78° C., solid NaHCO₃ (5 g) was added, and the reaction mixture was allowed to reach room temperature, while stirring. The reaction mixture was diluted with Et₂O (100 mL), and then washed with a mixture of brine (100 mL) and water (75 mL), dried (Na₂SO₄), and concentrated in vacuo. The obtained oil (22.2 g) was subjected to column chromatography (silicagel, heptane:EtOAc=3:1) to give 2,2,5,5-tetramethyl-[1,3]dioxane-4-carboxylic acid {4-[4-(2,3,4-tri-O-benzyl-α-D-xylopyranosyl)-butoxy]-2,6-dimethyl-phenyl}-amide (8.20 g, 57%, α:β˜2:1) as a colorless oil, followed by another impure batch of {4-[4-(2,3,4-tri-O-benzyl-α-D-xylopyranosyl)-butoxy]-2,6-dimethyl-phenyl}-amide (7.26 g). The latter batch contained 2,2,2-trichloroacetamide, which was partly removed by crystallization from a mixture of CH₂Cl₂ and heptane. The remaining oil (4.93 g) was purified by column chromatography (silicagel, heptane:EtOAc=3:1) to give another crop of {4-[4-(2,3,4-tri-O-benzyl-α-D-xylopyranosyl)-butoxy]-2,6-dimethyl-phenyl}-amide (3.90 g, 27%, α:β˜2:1) as a colorless oil. ¹H-NMR (CDCl₃) α anomer: δ (ppm)=7.76 (br s, 1H), 7.40-7.25 (m, 15H), 6.63 (s, 2H), 4.91 (d, J=10.8 Hz, 1H), 4.84 (d, J=10.8 Hz, 1H), 4.76 (d, J=10.8 Hz, 1H), 4.72-4.57 (m, 4H), 4.27 (s, 1H), 3.94-3.86 (m, 3H), 3.73 (d, J=11.7 Hz, 1H), 3.77-3.64 (m, 1H), 3.62-3.52 (m, 3H), 3.47-3.41 (m, 2H), 3.33 (d, J=11.7 Hz, 1H) 2.18 (s, 6H), 1.89-1.76 (m, 4H) 1.51 (s, 3H), 1.49 (s, 3H), 1.18 (s, 3H), 1.10 (s, 3H), visible signals from β anomer: δ (ppm)=6.60, 4.32 (d, J=7.5 Hz), 3.22-3.15 (m); ¹³C-NMR (CDCl₃) α anomer: δ (ppm)=167.9, 157.3, 138.7, 138.1, 138.0, 136.1 (2×), 128.16 (2×), 128.14 (2×), 128.07 (2×), 127.74 (2×), 127.69 (2×), 127.56 (2×), 125.86, 113.8 (2×), 99.1, 96,9, 81.3, 79.8, 78.1, 77.5, 75.7, 73.5, 73.2, 71.7, 67.7, 67.6, 60.0, 33.5, 29.7, 26.32, 26.28, 22.3, 19.6, 19.1 (2×), 19.0 (3 tertiary aromatic signals lay in the region 128.2-127.2. They could not be assigned due to presence of tertiary aromatic signals of the β-anomer).

2,2,5,5-Tetramethyl-[1,3]dioxane-4-carboxylic acid {4-[4-(α-D-xylopyranosyl)-butoxy]-2,6-dimethyl-phenyl}-amide. Under N₂ atmosphere, Pd on C (10% (w/w), 0.50 g, 0.47 mmol) and NaHCO₃ (1.00 g, 11.9 mmol) were added to a solution of 2,2,5,5-tetramethyl-[1,3]dioxane-4-carboxylic acid {4-[4-(2,3,4-tri-O-benzyl-α-D-xylopyranosyl)-butoxy]-2,6-dimethyl-phenyl}-amide (7.85 g, 10.1 mmol, α:β˜2:1) in EtOH (100 mL). The reaction flask was flushed with H₂ gas and the reaction mixture was stirred under H₂ atmosphere for 48 h. TLC analysis indicated that almost no starting material was converted. Therefore, the reaction mixture was filtered and the residue was washed with EtOH (4×20 mL). The filtrate and washings were combined, concentrated in vacuo and then EtOH (140 mL) was added. The resultant solution was treated with Pd on C (10% (w/w), 0.50 g, 0.47 mmol), CaCO₃ (1.70 g, 17.0 mmol) and hydrogenated for 3.5 h. TLC analysis indicated a complete reaction. The reaction mixture was treated with NaHCO₃ (1.00 g, 11.9 mmol), stirred for 0.5 h and filtered. The residue was washed with EtOH (4×20 mL) and the combined filtrate and washings were concentrated in vacuo to give an oil (6.23 g), which was purified by column chromatography (silicagel, CH₂Cl₂: MeOH=9:1) to give 2,2,5,5-tetramethyl-[1,3]dioxane-4-carboxylic acid {4-[4-(α-D-xylopyranosyl)-butoxy]-2,6-dimethyl-phenyl}-amide (4.49 g, 87%, α:β˜2:1) as a foam. ¹H-NMR (DMSO-d630 D₂O) α anomer: δ (ppm)=8.66 (br s, 1H), 6.60 (s, 2H), 4.58 (d, J=3.6 Hz, 1H), 4.18 (s, 1H), 3.92 (br q, J=5.6 Hz, 2H), 3.80-3.44 (m, 3H), 3.41-3.15 (m, 6H), 2.05 (s, 6H), 1.84-1.61 (m, 4H), 1.41 (s, 3H), 1.40 (s, 3H), 1.06 (s, 3H), 0.94 (s, 3H), OH signals are not visible; ¹³C-NMR (CDCl₃) α anomer: δ (ppm)=168.5, 157.2, 136.3 (2×), 125.7, 113.9 (2×), 99.2, 98.4, 77.4, 74.5, 72.0, 71.6, 69.9, 68.0, 67.4, 61.7, 33.4, 29.6, 26.28, 26.0, 22.3, 19.5, 19.0 (2×), 18.9 (the α-anomer is a mixture of two diastereomers, due to which many signals of corresponding carbon atoms in the separate diastereomers have a slightly different chemical shift); HRMS calcd for C₂₆H₄₂NO₉ (MH⁺), 512.2859, found 512.2842.

2,2,5,5-Tetramethyl-[1,3]dioxane-4-carboxylic acid {4-[4-(2,3,4-tri-O-acetyl-α-D-xylopyranosyl)-butoxy]-2,6-dimethyl-phenyl}-amide and 2,2,5,5-tetramethyl-[1,3]dioxane-4-carboxylic acid {4-[4-(2,3,4-tri-O-acetyl-β-D-xylopyranosyl)-butoxy]-2,6-dimethyl-phenyl}-amide. To a solution of 2,2,5,5-tetramethyl-[1,3]dioxane-4-carboxylic acid {4-[4-(α-D-xylopyranosyl)-butoxy]-2,6-dimethyl-phenyl}-amide (5.74 g, 11.2 mmol, α:β˜2:1) in pyridine (15 mL) was added Ac₂O (10 mL) at 0° C. The reaction mixture was stirred at 0° C. for 30 min, overnight at room temperature and then poured into a mixture of water and ice (200 mL) while stirring. After 2 h, the resulting mixture was extracted with CH₂Cl₂ (2×100 mL). The combined organic layers were washed with aqueous HCl (2M, 200 mL) and saturated aqueous NaHCO₃ solution (100 mL), dried (Na₂SO₄), and concentrated in vacuo. The remaining residue was purified by repetitive precise column chromatography (silica, heptane:EtOAc=1:1) to afford two fractions of 2,2,5,5-tetramethyl-[1,3]dioxane-4-carboxylic acid {4-[4-(2,3,4-tri-O-acetyl-α-D-xylopyranosyl)-butoxy]-2,6-dimethyl-phenyl}-amide (4.10 g, 57%, α:β˜12:1, 1.39 g, 20%, α:β˜1:1) all as colorless foams and a fraction of 2,2,5,5-tetramethyl-[1,3]dioxane-4-carboxylic acid {4-[4-(2,3,4-tri-O-acetyl-β-D-xylopyranosyl)-butoxy]-2,6-dimethyl-phenyl}-amide (1.25 g, 17%, α:β˜1:8) as a colorless foam. 2,2,5,5-Tetramethyl-[1,3]dioxane-4-carboxylic acid {4-[4-(2,3,4-tri-O-acetyl-α-D-xylopyranosyl)-butoxy]-2,6-dimethyl-phenyl}-amide: ¹H-NMR (CDCl₃) δ (ppm)=7.74 (br s, 1H), 6.59 (s, 2H), 5.47 (t, J=9.8 Hz, 1H), 4.99 (d, J=3.6 Hz, 1H), 4.94 (ddd, J=10.5, 9.5 5.9 Hz, 1H), 4.79 (dd, J=10.2, 3.6 Hz, 1H), 4.27 (s, 1H), 3.93 (t, J=6.0 Hz, 2H), 3.80-3.72 (m, 3H), 3.61 (t, J=10.7 Hz, 1H), 3.45 (dt, J=9.9, 6.0 Hz, 1H), 3.33 (d, J=11.4 Hz, 1H), 2.19 (s, 6H), 2.04 (s, 3H), 2.02 (s, 6H), 1.87-1.72 (m, 4H), 1.51 (s, 3H), 1.50 (s, 3H), 1.19 (s, 3H), 1.10 (s, 3H); ¹³C-NMR (CDCl₃) δ (ppm)=169.9, 169.64, 169.59, 167.9, 157.2, 136.2 (2×), 126.0, 113.8 (2×), 99.2, 95.6, 77.6, 71.7, 71.1, 69.7, 69.4, 68.1, 67.5, 58.4, 33.5, 29.7, 26.26, 26.21, 22.3, 20.94, 20.89, 20.85, 19.6, 19.1 (2×), 19.0; Anal. calcd for C₃₂H₄₇NO₁₂: C, 60.27; H, 7.43; N, 2.20, found: C, 60.21; H, 7.57; N, 2.41. 2,2,5,5-Tetramethyl-[1,3]dioxane-4-carboxylic acid {4-[4-(2,3,4-tri-O-acetyl-β-D-xylopyranosyl)-butoxy]-2,6-dimethyl-phenyl}-amide: ¹H-NMR (CDCl₃) δ (ppm)=7.74 (br s, 1H), 6.58 (s, 2H), 5.14 (t, J=9.8 Hz, 1H), 4.97-4.88 (m, 2H), 4.46 (d, J=6.6 Hz, 1H), 4.27 (s, 1H), 4.10 (dd, J=11.7, 5.1 Hz, 1H), 3.93-3.83 (m, 3H), 3.74 (d, J=11.7 Hz, 1H), 3.56-3.46 (m, 1H), 3.35 (dd, J=11.7, 9.2 Hz, 1H), 3.33 (d, J=11.4 Hz, 1H) 2.19 (s, 6H), 2.04 (s, 6H), 2.03 (s, 3H), 1.84-11.70 (m, 4H), 1.51 (s, 3H), 1.50 (s, 3H), 1.19 (s, 3H), 1.10 (s, 3 H); ¹³C-NMR (CDCl₃) δ (ppm)=169.7, 169.4, 169.0, 167.9, 157.2, 136.1 (2×), 125.9, 113.8 (2×), 100.5, 99.1, 77.5, 71.7, 71.4, 70.8, 69.1, 68.9, 67.4, 62.0, 33.4, 29.7, 26.3, 25.9, 22.3, 20.88, 20.85 (2×), 19.6, 19.1 (2×), 19.0; Anal. calcd for C₃₂H₄₇NO₁₂: C, 60.27; H, 7.43; N, 2.20, found: C, 60.25; H, 7.59; N, 2.31.

2,4-Dihydroxy-N-{4-[4-(2,3,4-tri-O-acetyl-α-D-xylopyranosyl)-butoxy]-2,6-dimethyl-phenyl}-3,3-dimethyl-butyramide. A mixture of HOAc (32 mL) and water (8 mL) was added to 2,2,5,5-tetramethyl-[1,3]dioxane-4-carboxylic acid {4-[4-(2,3,4-tri-O-acetyl-α-D-xylopyranosyl)-butoxy]-2,6-dimethyl-phenyl}-amide (3.70 g, 5.76 mmol, α:β˜12:1) under stirring. The reaction mixture was stirred for 24 h and then concentrated in vacuo. The resultant foam (3.90 g) was coevaporated from toluene (3×20 mL) and purified by column chromatography (silicagel, CH₂Cl₂:MeOH=19:1) to give 2,4-dihydroxy-N-{4-[4-(2,3,4-tri-O-acetyl-α-D-xylopyranosyl)-butoxy]-2,6-dimethyl-phenyl}-3,3-dimethyl-butyramide (3.26 g, 95%, α:β˜14:1) as a foam. ¹H-NMR (CDCl₃+D₂O) δ (ppm)=8.06 (br s, 1H), 6.60 (s, 2H), 5.46 (t, J=9.8 Hz, 1H), 4.99 (d, J=3.6 Hz, 1H), 4.94 (dt, J=9.9, 5.9 Hz, 1H), 4.79 (dd, J=9.9, 3.6 Hz, 1H), 4.15 (s, 1H), 3.93 (t, J=6.0 Hz, 2H), 3.80-3.71 (m, 2H), 3.61 (t, J=10.4 Hz, 1H), 3.56 (d, J=10.5 Hz, 1H), 3.50 (d, J=10.5 Hz, 1H), 3.44-341 (m, 1H), 2.18 (s, 6H), 2.04 (s, 3H), 2.03 (s, 6H), 1.86-1.75 (m, 4H), 1.10 (s, 3H), 1.02 (s, 3H), OH signals are not visible; ¹³C-NMR (CDCl₃) δ (ppm)=171.6, 169.9, 169.7, 169.6, 157.4, 136.2 (2×), 125.9, 113.9 (2×), 95.6, 78.2, 71.6, 71.1, 69.7, 69.5, 68.1, 67.5, 58.3, 39.6, 26.3, 26.2, 21.8, 20.95, 20.90, 20.86, 20.4, 19.1 (2×); Anal. calcd for C₂₉H₄₃NO₁₂: C, 58.28; H, 7.25; N, 2.34, found: C, 58.22; H, 7.23; N, 2.40.

2,4-Dihydroxy-N-{4-[4-(2,3,4-tri-O-acetyl-β-D-xylopyranosyl)-butoxy]-2,6-dimethyl-phenyl}-3,3-dimethyl-butyramide. A mixture of HOAc (9.6 mL) and water (2.4 mL) was added to 2,2,5,5-tetramethyl-[1,3]dioxane-4-carboxylic acid {4-[4-(2,3,4-tri-O-acetyl-β-D-xylopyranosyl)-butoxy]-2,6-dimethyl-phenyl}-amide (0.950 g, 1.49 mmol, α:β˜1:8) under stirring. The reaction mixture was stirred for 4 h and then concentrated in vacuo. The resultant foam (0.978 g) was coevaporated from toluene (3×10 mL) and purified by column chromatography (silicagel, CH₂Cl₂:MeOH=19:1) to give 2,4-dihydroxy-N-{4-[4-(2,3,4-tri-O-acetyl-β-D-xylopyranosyl)-butoxy]-2,6-dimethyl-phenyl}-3,3-dimethyl-butyramide (0.805 g, 96%, α:β˜1:7) as a foam. ¹H-NMR (CDCl₃+D₂O) δ (ppm)=8.07 (br s, 1H), 6.58 (s, 2H), 5.13 (t, J=8.6 Hz, 1H), 4.99-4.87 (m, 2H), 4.46 (d, J=6.9 Hz, 1H), 4.15 (s, 1H), 4.06 (dd, J=11.7, 5.1 Hz, 1H), 3.95-3.82 (m, 1H), 3.88 (t, J=5.9 Hz, 2H), 3.58-3.46 (m, 3H), 3.35 (dd, J=11.7, 8.7 Hz, 1H), 2.18 (s, 6H), 2.04 (s, 3H), 2.03 (s, 6H), 1.86-1.70 (m, 4H), 1.10 (s, 3H), 1.01 (s, 3H), OH signals are not visible; ¹³C-NMR (CDCl₃) δ (ppm)=171.7, 169.8, 169.5, 169.1, 157.4, 136.2 (2×), 125.9, 113.8 (2×), 100.5, 78.2, 71.6, 71.5, 70.8, 69.1, 68.9, 67.4, 62.0, 39.6, 26.3, 25.9, 21.8, 20.91, 20.87 (2×), 20.4, 19.1 (2×); Anal. calcd for C₂₉H₄₃NO₁₂: C, 58.28; H, 7.25; N, 2.34, found: C, 58.09; H, 7.38; N, 2.38.

### Example 9

Synthesis of N-(2,6-dimethyl-4-pentyloxy-phenyl)-2,4-dihydroxy-3,3-dimethyl-butyramide (AA)

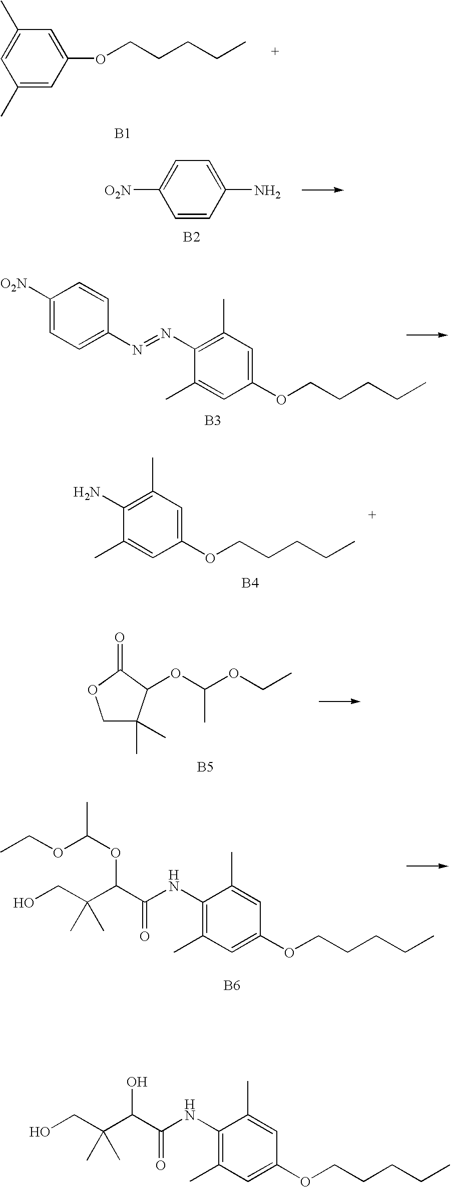

(2,6-Dimethyl-4-pentyloxy-phenyl)-(4-nitro-phenyl)-diazene. A mixture of p-nitro-aniline (19.4 g, 0.141 mol), water (50.5 mL) and concentrated HCl (50.5 mL) was heated until a clear solution was obtained and then cooled to 0° C., using an ice-salt bath. A solution of NaNO₂ (14.4 g, 0.209 mol) in water (31 mL) was added dropwise to the cold mixture at such a rate that the temperature remained below 5° C. The addition of the sodium nitrite solution was stopped when a positive reaction on a iodine/starch paper was obtained. The obtained solution was kept cold (0° C.) and added dropwise in 0.5 h to a solution of 3,5-dimethyl-1-pentyloxy-benzene (27 g, 0.141 mol, prepared according to: de Benneville, P. L.; Bock, L. H., patent U.S. Pat. No. 2499214, 1947) in AcOH (500 mL). At the beginning of the addition, the solution of 3,5-dimethyl-1-pentyloxy-benzene was cooled to 15° C. with an ice bath. During the addition the temperature dropped to 8° C. AcOH (500 mL) was added to the reaction mixture, under cooling in an ice bath (reaction mixture temperature was 10° C.), until an almost homogeneous solution was obtained. Water (20 mL) was added, and the reaction mixture was set aside in the refrigerator. After 3 days the mixture was filtered and the obtained crystalline material was washed with aqueous AcOH (50%, 3×130 mL). The filtrate and washings were combined and set aside in the refrigerator. The residue was washed with water (3×100 mL) and air dried to give (2,6-dimethyl-4-pentyloxy-phenyl)-(4-nitro-phenyl)-diazene (22.0 g, 46%) as a red brown crystalline material. A second and third crop of (2,6-dimethyl-4-pentyloxy-phenyl)-(4-nitro-phenyl)-diazene (6.1 g, 13% and 2.0 g, 4%) were obtained, using the same procedure as described above, after a 3 days interval. A fourth crop (2.1 g, 4%) was isolated after standing for 5 days at room temperature. The third and fourth crop (sticky material) were combined and recrystallized from 2-propanol to give pure (2,6-dimethyl-4-pentyloxy-phenyl)-(4-nitro-phenyl)-diazene (3.1 g, 6%). Combined yield of (2,6-dimethyl-4-pentyloxy-phenyl)-(4-nitro-phenyl)-diazene was 31.1 g (65%). ¹H-NMR (CDCl₃) δ (ppm)=8.32 (d, J=9.0 Hz, 2H), 7.89 (d, J=9.0 Hz, 2H), 6.67 (s, 2H), 4.01 (t, J=6.6 Hz, 2H), 2.55 (s, 6H), 1.81 (quintet, J=6.9 Hz, 2H), 1.51-1.34 (m, 4H), 0.95 (t, J=7.1 Hz, 3H); ¹³C-NMR (CDCl₃) δ (ppm)=160.8, 156.5, 147.9, 143.6, 137.2 (2×), 124.6 (2×), 122.6 (2×), 115.3 (2×), 68.1, 28.9, 28.1, 22.4, 21.1, 14.0; HRMS calcd for C_{??}H_{??}N_{?}O_{?} (M⁺):, found:; Anal. calcd for C₁₉H₂₃N₃O₃: C, 66.84; H, 6.79; N, 12.31, found: C, 67.06; H, 6.56; N, 12.23.

2,6-Dimethyl-4-pentyloxy-phenylamine. To a stirred mixture of sodium dithionite (112 g, 0.644 mol) in EtOH (660 mL) and water (660 mL) was added portion wise (2,6-dimethyl-4-pentyloxy-phenyl)-(4-nitro-phenyl)-diazene (22.0 g, 0.0644 mol) over a 10 min period. The reaction mixture was stirred under reflux for 1 h and then allowed to reach room temperature. A mixture was obtained with a slightly yellow color. The reaction mixture was reduced to half its volume and then extracted with Et₂O (1×600 mL, 2×100 mL). The combined organic layers were washed with brine (300 mL), dried (Na₂SO₄) and concentrated in vacuo. The remaining residue was purified by means of column chromatography (silica, heptane:EtOAc=4:1) to give 2,6-dimethyl-4-pentyloxy-phenylamine (10.7 g, 80%) as a purple thin oil. Although LC/MS showed no contamination on TLC a small impurity was visible. ¹H-NMR (CDCl₃) δ (ppm)=6.55 (s, 2H), 3.86 (t, J=6.6 Hz, 2H), 3.32 (br s, 2H) 2.15 (s, 6H), 1.73 (quintet, J=7.0Hz, 2H), 1.47-1.35 (m, 4H), 0.92 (t, J=7.1 Hz, 3H); ¹³C-NMR (CDCl₃) δ (ppm)=151.5, 136.2 (2×), 123.1, 114.7 (2×), 68.5, 29.1, 28.2, 22.4, 17.9 (2×), 14.0; HRMS calcd for C₁₃H₂₁NO (M⁺): 207.1623, found 207.1623.

N-(2,6-Dimethyl-4-pentyloxy-phenyl)-2-(1-ethoxy-ethoxy)-4-hydroxy-3,3-dimethyl-butyramide. A solution of 2,6-dimethyl-4-pentyloxy-phenylamine (4.44 g, 21.4 mmol) in dry DMF (22 mL) was treated with NaH (60% (w/w) dispersion in mineral oil, 0.856 g, 21.4 mmol) and stirred for 5 min under an argon atmosphere. Then 3-(1-ethoxy-ethoxy)-4,4-dimethyl-dihydro-furan-2-one (4.32 g, 21.4 mmol, prepared according to: Dujardin, G.; Rossignol, S.; Brown, E. Synthesis, 1998, 5, 763-770) was added to the mixture. The resultant mixture was stirred overnight, then poured into a mixture of water/ice (200 mL) and brine (50 mL) and extracted with Et₂O (2×100 mL). The combined organic layers were washed with brine (3×100 mL), dried (Na₂SO4) and concentrated in vacuo to give a dark brown oil (7.50 g). Column chromatography (silica, heptane:EtOAc=4:1, later 2:1) of the oil gave first a crop of 2,6-dimethyl-4-pentyloxy-phenylamine (1.88 g, 42%), followed by N-(2,6-dimethyl-4-pentyloxy-phenyl)-2-(1-ethoxy-ethoxy)-4-hydroxy-3,3-dimethyl-butyramide (2 partly separated diastereomers which were combined, 5.06 g, 58%) as a yellow oil. ¹H-NMR (CDCl₃) (mixture of diastereomers), major diastereomer: δ (ppm)=7.68 (s, 1H), 6.63 (s, 2H), 4.74 (q, J=5.1 Hz, 1H), 4.19 (s, 1H), 3.91 (t, J=6.5 Hz, 2H), 3.65-3.54 (m, 4H), 3.31 (dd, J=13.7, 10.4 Hz, 1H), 2.20 (s, 6H), 1.76 (quintet, J=6.9 Hz, 2H), 1.44-1.38 (m, 7H), 1.25 (t, J=7.1, 3H), 1.09 and 1.06 (2s, 6H), 0.93 (t, J=7.1 Hz, 3H), peaks not overlapped by major diastereomer: δ (ppm)=7.99 (s), 6.59 (s), 4.63 (q, J=5.1 Hz), 3.98 (s), 3.77 (m), 3.50-3.37 (m), 2.21(s), 1.16 (t, J=7.1 Hz), 0.97 (s); ¹³C-NMR (CDCl₃) (mixture of diastereomers), major diastereomer: δ (ppm)=170.7, 157.9, 136.2 (2×), 125.7, 114.1 (2×), 100.6, 81.1, 70.1, 67.9, 62.5, 39.9, 28.8, 28.1, 22.3, 21.6, 20.7, 20.4, 19.3 (2×), 15.0, 13.9, peaks not overlapped by major diastereomer: δ (ppm)=171.5, 157.7,136.3, 125.9, 113.9, 103.8, 83.5, 70.3, 63.7, 40.8, 23.4, 20.6, 19.2, 19.1, 15.3; HRMS calcd for C₂₃H₄₀NO₅ (MH⁺): 410.2906, found: 410.2919.

N-(2,6-Dimethyl-4-pentyloxy-phenyl)-2,4-dihydroxy-3,3-dimethyl-butyramide. N-(2,6-dimethyl-4-pentyloxy-phenyl)-2-( 1-ethoxy-ethoxy)-4-hydroxy-3,3-dimethyl-butyramide (5.00 g, 12.2 mmol) was dissolved in a mixture of HOAc (40 mL) and water (10 mL), set aside for 2 h. and concentrated in vacuo (10 mm Hg, 37° C.). The resultant oil was concentrated from toluene (2×30 mL) and then crystallized from iPr₂O (30 mL) to give N-(2,6-dimethyl-4-pentyloxy-phenyl)-2,4-dihydroxy-3,3-dimethyl-butyramide (3.56 g, 86%) as beige crystals. mp: 101-102.5° C.; ¹H-NMR (CDCl₃) δ=7.99 (s, 1H, NH), 6.62 (s, 2H), 4.23 (d, J=4.8 Hz, 1H, on exchange with D₂O: s, 1H), 3.90 (t, J=6.6 Hz, 2H), 3.70 (br s, 1H, OH), 3.65 (dd, J=11.1, 5.7 Hz, 1H, on exchange with D₂O: d, J=11.1 Hz), 3.56 (dd, J=11.1, 5.7 Hz, 1H, on exchange with D₂O: d, J=11.1 Hz), 3.08 (br s, 1H, OH), 2.20 (s, 1H), 1.75 (quintet, J=6.9 Hz, 2H), 1.47-1.31 (m, 4H), 1.14 and 1.04 (2s, 6H), 0.92 (t, J=7.2 Hz, 3H); ¹³C-NMR (CDCl₃) δ=172.2, 157.9, 136.4 (2×), 125.8, 114.0 (2×), 78.2, 71.6, 68.0, 39.5, 28.9, 28.1, 22.4, 21.5, 20.3, 18.9 (2×), 14.0; HRMS calcd for C₁₉H₃₂NO₄ (MH⁺): 338.2331, found: 338.2337.

### Example 10

2,4-dihydroxy-N-[4-(6-hydroxy-5,5-dimethyl-hexyloxy)-2,6-dimethyl-phenyl]-3,3-dimethyl-butyramide (AC)

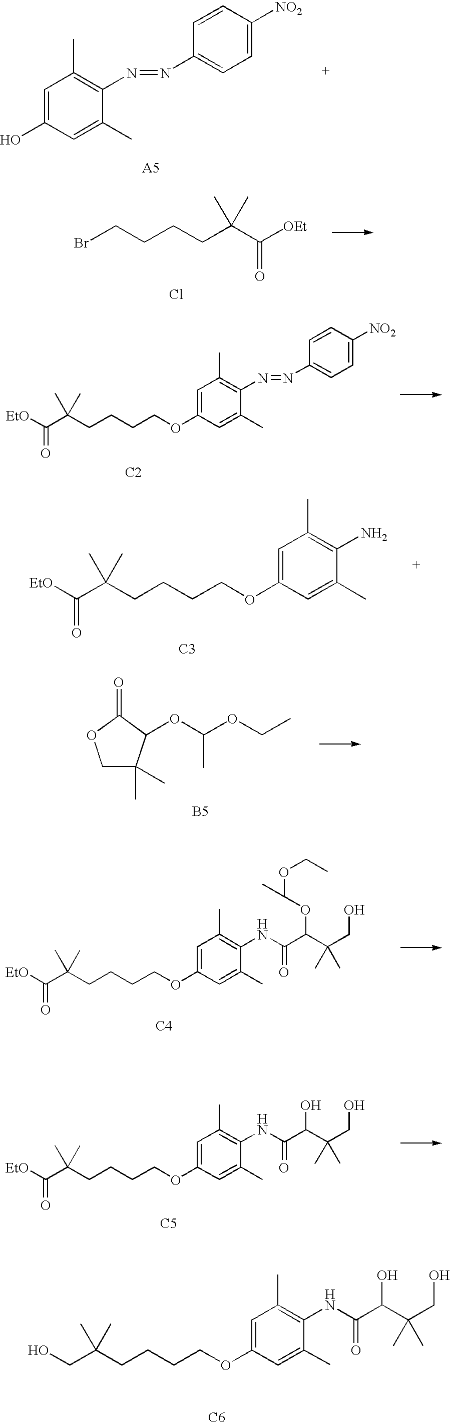

6-[3,5-Dimethyl-4-(4-nitro-phenylazo)-phenoxy]-2,2-dimethyl-hexanoic acid ethyl ester. A solution of 3,5-dimethyl-4-(4-nitro-phenylazo)-phenol (10 g, 36.9 mmol, prepared according to: Smith, L. I.; Irwin, W. B., J. Am. Chem. Soc., 1941, 63, 1036-1043) and 6-bromo-2,2-dimethyl-hexanoic acid ethyl ester (9.26 g, 36.9 mmol, prepared according to: Ackerley, N.; Brewster, A. G.; Brown, G. R.; Clarke, D. S.; Foubister, A. J. J Med. Chem., 1995, 38; 1608-1628) in DMSO (50 mL) was treated with K₂CO₃ (5.09 g, 36.9 mmol). The dark black-blue reaction mixture was stirred for 3 days at room temperature and a crystalline mass appeared. The reaction mixture was poured into a mixture of water and ice (300 mL) and the resulting mixture was filtered, washed with water (300 mL), and air dried to give a crystalline mass, which was recrystallized from EtOH (100 mL) to give 6-[3,5-dimethyl-4-(4-nitro-phenylazo)-phenoxy]-2,2-dimethyl-hexanoic acid ethyl ester (11.5 g, 71%) as dark red-brown needles. mp 89-90° C.; ¹H-NMR (CDCl₃) δ (ppm)=8.34 (d, J=9.0 Hz, 2H), 7.92 (d, J=9.0 Hz, 2H), 6.67 (s, 2H), 4.13 (q, J=7.2 Hz, 2H), 4.01 (t, J=6.5 Hz, 2H), 2.56 (s, 6H), 1.79 (q, J=7.0 Hz, 2H), 1.63-1.58 (m, 2H), 1.47-1.37 (m, 2H), 1.25 (t, J=7.2 Hz, 3H), 1.20 (s, 6H); ¹³C-NMR (CDCl₃) δ (ppm)=177.8, 160.7, 156.5, 147.9, 143.7, 137.2 (2×), 124.7 (2×), 122.6 (2×), 115.3 (2×), 67.8, 60.2, 42.1, 40.3, 29.6, 25.1 (2×), 21.5, 21.1 (2×), 14.2; Anal. calcd for C₂₄H₃₁N₃O₅: C, 65.29; H, 7.08; N, 9.52, found: C, 65.62; H, 7.01; N, 9.71.

6-(4-Amino-3,5-dimethyl-phenoxy)-2,2-dimethyl-hexanoic acid ethyl ester. A mixture of 6-[3,5-dimethyl-4-(4-nitro-phenylazo)-phenoxy]-2,2-dimethyl-hexanoic acid ethyl ester (10.75 g, 24.4 mmol) and sodium dithionite (44.9 g, 0.244 mol) in EtOH (250 mL) and water (250 mL) was stirred under reflux for 1 h, and then allowed to reach room temperature. An orange colored mixture was obtained, which was reduced to 200 mL by means of concentration in vacuo and then extracted with Et₂O (1×300 mL, 2×100 mL). The combined organic layers were washed with brine (150 mL), dried (Na₂SO₄) and concentrated in vacuo. The remaining residue was purified by column chromatography (silica, heptane:EtOAc=2:1) to give 6-(4-amino-3,5-dimethyl-phenoxy)-2,2-dimethyl-hexanoic acid ethyl ester (6.74 g, 90%) as a brownish thin oil, which solidified when kept at −20° C. An analytical sample of 6-(4-amino-3,5-dimethyl-phenoxy)-2,2-dimethyl-hexanoic acid ethyl ester (0.583 g, light red brown crystals) was obtained on crystallization of 0.727 g from a mixture of EtOH and water (1:1). mp 32-34° C.; ¹H-NMR (CDCl₃) δ (ppm)=6.54 (s, 2H), 4.11 (q, J=7.2 Hz, 2H), 3.85 (t, J=6.5 Hz, 2H), 3.23 (br s, 2H), 2.15 (s, 6H), 1.70 (quintet, J=6.9 Hz, 2H), 1.60-1.54 (m, 2H), 1.43-1.32 (m, 2H), 1.23 (t, J=7.2 Hz, 3H), 1.67 (s, 6H); ¹³C-NMR (CDCl₃) δ (ppm)=177.8, 151.3, 136.3, 123.0 (2×), 114.7 (2×), 68.2, 60.1, 42.1, 40.3, 29.8, 25.0 (2×), 21.4, 17.8 (2×), 14.1; Anal. calcd for C₁₈H₂₉NO₃: C, 70.32; H, 9.51; N, 4.56, found: C, 70.50; H, 9.59; N, 4.31.

6-{4-[2-(1-Ethoxy-ethoxy)-4-hydroxy-3,3-dimethyl-butyrylamino]-3,5-dimethyl-phenoxy}-2,2-dimethyl-hexanoic acid ethyl ester. A solution of 6-(4-amino-3,5-dimethyl-phenoxy)-2,2-dimethyl-hexanoic acid ethyl ester (5.42 g, 17.7 mmol) in DMF (30 mL) was treated with NaH (60% (w/w) dispersion in mineral oil, 0.76 g, 19 mmol) and stirred for 30 min under N₂ atmosphere. 3-(1-ethoxy-ethoxy)-4,4-dimethyl-dihydro-furan-2-one (3.57 g, 17.7 mmol, prepared according to: Dujardin, G.; Rossignol, S.; Brown, E. Synthesis, 1998, 5, 763-770) was added to the reaction mixture and stirring was continued for another 6 h. Then, the mixture was poured into a mixture of ice (100 mL), water (100 mL), and saturated aqueous NaHCO₃ (100 mL). After 1 h, the mixture was extracted with Et₂O (3×100 mL) and the combined organic layers were washed with brine (3×75 mL), dried, and concentrated in vacuo to give a dark brown oil (7.93 g), which was subjected to column chromatography (silica, heptane:EtOAc=2:1). The first eluting fraction was 6-(4-amino-3,5-dimethyl-phenoxy)-2,2-dimethyl-hexanoic acid ethyl ester (1.59 g). Continued elution gave 6-{4-[2-(1-ethoxy-ethoxy)-4-hydroxy-3,3-dimethyl-butyrylamino]-3,5-dimethyl-phenoxy}-2,2-dimethyl-hexanoic acid ethyl ester (3.47 g, 39%, 2 diastereomeric sets (ratio ˜3:1)) as a brown oil, followed by a mixture of 6-{4-[2-(1-ethoxy-ethoxy)-4-hydroxy-3,3-dimethyl-butyrylamino]-3,5-dimethyl-phenoxy}-2,2-dimethyl-hexanoic acid ethyl ester and an unidentified compound (1.15 g). ¹H-NMR (CDCl₃) δ (ppm)=7.67 (s, 1H), 6.62 (s, 2H), 4.74 (q, J=5.1 Hz, 1H), 4.19 (s, 1H), 4.11 (q, J=7.1 Hz, 2H), 3.91 (t, J=6.5 Hz, 2H), 3.62 (q, J=7.0 Hz, 2H), 3.57 (d, J=11.4 Hz, 1H), 3.31 (d, J=11.4 Hz, 1H), 2.20 (s, 6H), 1.73 (quintet, J=6.9 Hz, 2H), 1.60-1.54 (m, 2H), 1.44 (d, J=5.1 Hz, 3H), 1.42-1.37 (m, 2H), 1.25 (t, J=7.0 Hz, 3H), 1.24 (t, J=7.0 Hz, 3H), 1.17 (s, 6H), 1.09 (s, 3H), 1.07 (s, 3H), peaks not overlapped by major diastereomer: δ (ppm)=8.02 (s), 6.60 (s), 4.65 (q, J=5.1 Hz), 2.32 (s); ¹³C-NMR (CDCl₃) δ (ppm)=177.8, 170.7, 157.8, 136.2 (2×), 125.8, 114.2 (2×), 100.6, 81.1, 70.1, 67.6, 62.6, 60.1, 42.0, 40.2, 39.9, 29.6, 25.0 (2×), 21.6, 21.4, 20.7, 20.4, 19.3 (2×), 15.0, 14.1, peaks not overlapped by major diastereomer: δ (ppm)=171.5, 157.6, 136.4, 126.0, 114.0, 103.8, 83.6, 70.3, 63.7, 40.8, 23.5, 20.6, 19.1 17.8, 15.3; HRMS calcd for C₂₈H₄₈NO₇ (MH⁺), 510.3431, found: 510.3385.

6-[4-(2,4-Dihydroxy-3,3-dimethyl-butyrylamino)-3,5-dimethyl-phenoxy]-2,2-dimethyl-hexanoic acid ethyl ester. A solution of 6-{4-[2-(1-ethoxy-ethoxy)-4-hydroxy-3,3-dimethyl-butyrylamino]-3,5-dimethyl-phenoxy}-2,2-dimethyl-hexanoic acid ethyl ester (2.98 g, 5.86 mmol) in HOAc (28 mL) and water (7 mL) was stirred for 4 h and then concentrated in vacuo. The resultant dark green oil (3.30 g) was coevaporated from toluene (2×20 mL) and purified by column chromatography (silica, heptane:EtOAc=1:2) to give 6-[4-(2,4-dihydroxy-3,3-dimethyl-butyrylamino)-3,5-dimethyl-phenoxy]-2,2-dimethyl-hexanoic acid ethyl ester (1.94 g, 76%) as a light brown oil. ¹H-NMR (CDCl₃) δ (ppm)=8.12 (s, 1H), 6.59 (s, 2H), 4.12 (s, 1H), 4.11 (q, J=7.1 Hz, 2H), 3.88 (t, J=6.3 Hz, 2H), 3.50 (d, J=11.4 Hz, 1H), 3.47 (d, J=11.4 Hz, 1H), 2.17 (s, 6H), 1.72 (quintet, J=7.0 Hz, 2H), 1.60-1.54 (m, 2H), 1.42-1.34 (m, 2H), 1.24 (t, J=7.1 Hz, 3H), 1.17 (s, 6H), 1.07 (s, 3H), 1.00 (s, 3H); ¹³C-NMR (CDCl₃) δ (ppm)=178.1, 172.3, 157.8, 136.4 (2×), 125.9, 114.0 (2×), 78.1, 71.6, 67.6, 60.3, 42.1, 40.3, 39.5, 29.7, 25.1 (2×), 21.5, 21.4, 20.2, 18.9 (2×), 14.2; HRMS calcd for C₂₄H₄₀NO₆ (MH⁺), 438.2856, found: 438.2833.

2,4-Dihydroxy-N-[4-(6-hydroxy-5,5-dimethyl-hexyloxy)-2,6-dimethyl-phenyl]-3,3-dimethyl-butyramide. A solution of 6-[4-(2,4-dihydroxy-3,3-dimethyl-butyrylamino)-3,5-dimethyl-phenoxy]-2,2-dimethyl-hexanoic acid ethyl ester (4.48 g, 10.25 mmol) in 1,2-dimethoxyethane (DME, 90 mL) was added dropwise to a suspension of LiAlH₄ (1.67 g, 43.8 mmol) in DME (450 mL) over a period of 30 min under N₂ atmosphere at 0° C. After stirring the reaction mixture for 1 h at 0° C., water (7 mL) was added dropwise over a period of 30 min under an N₂ atmosphere at 0° C. The resultant mixture was treated with Na₂SO₄ (˜40 g) and then filtered through a layer of Na₂SO₄ (1 cm) in a glassfilter. The residue was washed with DME (5×100 mL) and the combined filtrates were concentrated in vacuo to give a light brown thick oil (3.18 g), which was purified by column chromatography (silica, heptane:EtOAc=3:1) to give 2,4-dihydroxy-N-[4-(6-hydroxy-5,5-dimethyl-hexyloxy)-2,6-dimethyl-phenyl]-3,3-dimethyl-butyramide (2.67 g, 67%) as an almost colorless foam. ¹H-NMR (CDCl₃+D₂O) δ (ppm)=8.20 (s, 1H), 6.59 (s, 2H), 4.05 (s, 1H), 3.89 (t, J=6.5 Hz, 2H), 3.44 (s, 2H), 3.25 (s, 2H), 2.14 (s, 6H), 1.71 (quintet, J=6.8 Hz, 2H), 1.43-1.33 (m, 2H), 1.30-1.23 (m, 2H), 1.02 (s, 3H), 0.97 (s, 3H), 0.85 (s, 6H).; ¹³C-NMR (CDCl₃) δ (ppm)=172.8, 157.7, 136.4 (2×), 125.9, 113.9 (2×), 77.7, 71.6, 71.3, 67.8, 39.4, 38.2, 35.0, 30.0, 23.8 (2×), 21.3, 20.3 (2×), 18.8 (2×).; HRMS calcd for C₂₂H₃₇NO₅ (M⁺): 395.26644, found: 395.2671.

### Example 11

6-4-[(2,4-dihydroxy-3,3-dimethylbutanoyl)amino]-3,5-dimethylphenoxy-2,2-dimethylhexanoic acid (AE)

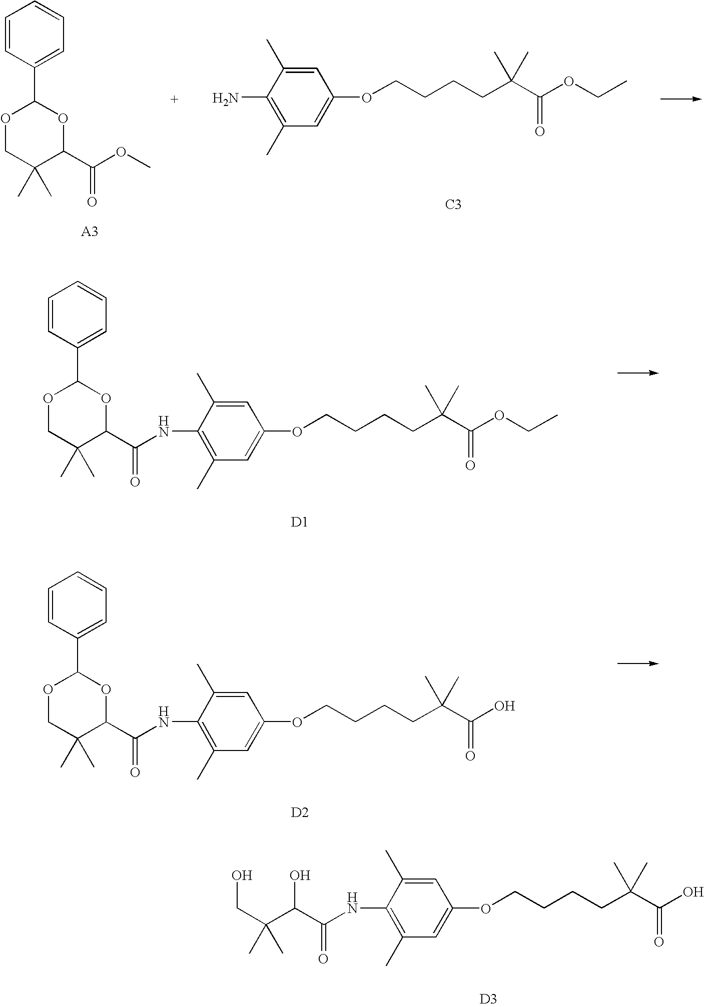

Ethyl 6-(4-[(5,5-dimethyl-2-phenyl-1,3-dioxan-4-yl)carbonyl]amino-3,5-dimethylphenoxy)-2,2-dimethylhexanoate. A mixture of 5,5-dimethyl-2-phenyl-[1,3]-dioxane-4-carboxylic acid methyl ester (A3, 2.22 g, 4.0 mmol), LiOH.H₂O(0.56 g, 13.3 mmol), water (10 drops), and MeOH (50 mL) was stirred at 40° C. for 18 h. The reaction mixture was concentrated in vacuo and coevaporated from toluene (4×50 mL), yielding a white solid. Toluene (100 mL) was added and the mixture was concentrated in vacuo to a smaller volume (˜50 mL). SOCl₂ (0.80 mL, 11 mmol) was added, and the reaction mixture was stirred at room temperature for 1 h. Then, the mixture was cooled to −10° C., and pyridine (˜8 mL) was added, causing a yellow solid material to appear and clot together. The reaction flask was flushed with argon gas, and a solution of 6-(4-amino-3,5-dimethyl-phenoxy)-2,2-dimethyl-hexanoic acid ethyl ester (C3, 2.52 g, 7.4 mmol) in pyridine (˜10 mL) was added fast. After stirring at room temperature for 2 h, the reaction mixture was poured out into a water/ice mixture (200 mL), which was then stirred vigorously for 10 min. The resulting mixture was extracted with Et₂O (1×100 mL, 2×50 mL), and the combined organic layers were washed with aq. NaCl (10%, 100 mL), brine (100 mL), dried (Na₂SO₄) and concentrated in vacuo, yielding a thick yellow-brown oil (4.08 g). This crude product was purified by column chromatography (silica, heptane/EtOAc=2:1) and stripped with CH₂Cl₂ (100 mL) giving ethyl 6-(4-[(5,5-dimethyl-2-phenyl-1,3-dioxan-4-yl)carbonyl]amino-3,5-dimethylphenoxy)-2,2-dimethylhexanoate (3.31 g, containing 8% (w/w) CH₂Cl₂, 78%) as a slightly brownish oil. ¹H-NMR (CDCl₃) δ (ppm)=7.71 (s, 1H), 7.53-7.50 (m, 2H), 7.42-7.37 (m, 3H), 6.56 (s, 2H), 5.59 (s, 1H), 4.30 (s, 1H), 4.09 (q, J=7.0 Hz, 2H), 3.87 (t, J=6.5 Hz, 2H), 3.78 (d, J=11.4 Hz, 1H), 3.72 (d, J=11.4 Hz, 1H), 2.18 (s, 6H), 1.72 (quinet, J=6.8 Hz, 2H), 1.59-1.54 (m, 2H), 1.42-1.34 (m, 2H), 1.32 (s, 3H), 1.23 (t, J=7.0 Hz, 3H), 1.17 (s, 3H), 1.16 (s, 6H); ¹³C-NMR (CDCl₃) δ (ppm)=183.4, 167.3, 157.4, 137.5, 136.2 (2×), 129.1, 128.2 (2×), 125.9 (2×), 125.7, 113.9 (2×), 101.4, 84.1, 78.7, 67.7, 60.3, 42.3, 40.5, 33.7, 29.8, 25.3 (2×), 22.1, 21.7, 19.8, 19.2 (2×), 14.5; HRMS calcd for C₃₁H₄₃NO₆ (M⁺): 525.3032, found 525.3044.

6-(4-[(5,5-Dimethyl-2-phenyl-1,3-dioxan-4-yl)carbonyl]amino-3,5-dimethylphenoxy)-2,2-dimethylhexanoic acid. Ethyl 6-(4-[(5,5-dimethyl-2-phenyl-1,3-dioxan-4-yl)carbonyl]amino-3,5-dimethylphenoxy)-2,2-dimethyl-hexanoate (11.05 g, 95% pure, 20.0 mmol) was dissolved in EtOH (300 mL) by heating. Water (100 mL) was added to the solution, followed by LiOH.H₂O (3.72 g, 89 mmol). The reaction mixture was refluxed for 38 h and allowed to cool to room temperature. The solvent was removed in vacuo, yielding a yellow sludge. The crude material was dissolved in water (200 mL), and CH₂Cl₂ (200 mL) was added, giving a milk-like suspension. Addition of aq. HCl (2 M, 200 mL) caused phase separation, and the aqueous layer was extracted with CH₂Cl₂ (1×200 mL, 1×100 mL). The combined organic layers were washed with water (200 mL), and saturated NaHCO₃ (200 mL), dried (Na₂SO₄, minimal amount), and concentrated in vacuo, to give 6-(4-[(5,5-dimethyl-2-phenyl-1,3-dioxan-4-yl)carbonyl]amino-3,5-dimethyl-phenoxy)-2,2-dimethylhexanoic acid (9.66 g, 97%) as a white foam. ¹H-NMR (CDCl₃) δ (ppm)=7.72 (s, 1H), 7.53-7.49 (m, 2H), 7.42-7.35 (m, 3H), 6.57 (s, 2H), 5.60 (s, 1H), 4.31 (s, 1H), 3.89 (t, J=6.5 Hz, 2H), 3.78 (d, J=11.1 Hz, 1H), 3.72 (d, J=11.4 Hz, 1H), 2.17 (s, 6H), 1.73 (quintet, J=6.8 Hz, 2H), 1.61-1.36 (m, 4H), 1.32 (s, 3H), 1.19 (s, 6H), 1.17 (s, 3H). The CO₂H signal is not visible; ¹³C-NMR (CDCl₃) δ (ppm)=183.4, 167.5, 157.5, 137.5, 136.2 (2×), 129.0, 128.2 (2×), 125.9 (2×), 125.6, 114.0 (2×), 101.4, 84.1, 78.7, 67.7, 42.2, 40.2, 33.7, 29.9, 25.1 (2×), 22.1, 21.6, 19.8, 19.1 (2×);

6-4-[(2,4-Dihydroxy-3,3-dimethylbutanoyl)amino]-3,5-dimethylphenoxy-2,2-dimethylhexanoic acid. A flask with a solution of 6-(4-[(5,5-dimethyl-2-phenyl-1,3-dioxan-4-yl)carbonyl]amino-3,5-dimethylphenoxy)-2,2-dimethylhexanoic acid (8.96 g, 18.0 mmol) in EtOH (100 mL) was flushed with N₂ gas. Pd on C (5% (w/w), ˜0.03 g, ˜0.14 mmol) was added, and the flask was flushed with H₂ gas. After stirring at room temperature for 15 h, TLC analysis showed no conversion and the reaction mixture was grey, indicating precipitation of starting material. EtOH (100 mL) was added and the mixture was slightly heated to dissolve the precipitate. The H₂ atmosphere was restored and stirring was continued for 1 day. However, the starting material had precipitated again and TLC showed no conversion. The reaction mixture was warmed to 35° C. to prevent precipitation, and Pd on C (5% (w/w), ˜0.03 g, ˜0.14 mmol) was added. The flask was flushed with H₂ gas again, and after 5 d of stirring at room temperature, TLC analysis showed a complete reaction. The reaction mixture was filtered through two stacked folded filter papers. The clear, light yellow filtrate was concentrated in vacuo to give 6-4-[(2,4-dihydroxy-3,3-dimethylbutanoyl)amino]-3,5-dimethylphenoxy-2,2-dimethylhexanoic acid (7.14 g, 96%) as a hard white foam. ¹H-NMR (CD₃OD) δ (ppm)=6.63 (s, 2H), 4.09 (s, 1H), 3.92 (t, J=6.3 Hz, 2H), 3.56 (d, J=11.0 Hz, 1H), 3.47 (d, J=11.0 Hz, 1H), 2.18 (s, 6H), 1.72 (quintet, J=6.8 Hz, 2H), 1.61-1.38 (m, 4H), 1.17 (s, 6H), 1.06 (s, 3H), 1.05 (s, 3H), the NH and OH signals are not visible; ¹³C-NMR (CD₃OD) δ (ppm)=182.0, 175.6, 159.4, 138.1 (2×), 128.1, 115.1 (2×), 77.9, 70.7, 68.9, 43.2, 41.7, 40.8, 31.0, 25.8 (2×), 22.9, 21.6, 21.3, 19.2 (2×); Anal. calcd for C₂₉H₃₉NO₆: C, 70.00; H, 7.90; N, 2.81, found: C, 69.54; H, 7.88; N, 2.77.

### Example 12

2,4-Dihydroxy-3,3-dimethyl-N-pyridin-3ylmethyl-butyramide (AB)

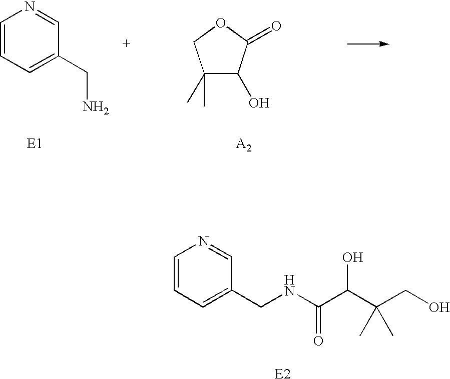

2,4-Dihydroxy-3,3-dimethyl-N-pyridin-3ylmethyl-butyramide. A solution of 3-(aminomethyl)-pyridine (5.00 g, 4.72 mL, 43.7 mmol) and (D,L)-pantolactone (5.68 g, 43.7 mmol) in absolute EtOH (50 mL) was stirred under reflux for 5 days and then concentrated in vacuo to give a solid which was recrystallized from EtOH/iPr₂O to give 2,4-dihydroxy-3,3-dimethyl-N-pyridin-3ylmethyl-butyramide (9.26 g, 84%) as colorless crystals. mp 120-121.5 ° C. ¹H-NMR (DMSO-d6) δ (ppm)=8.50 (d, J=2.0 Hz, 1H), 8.43 (dd, J=2.0, 4.8 Hz, 1H), 8.36 (t, J=6.2 Hz, 1H, disappears on exchange with D₂O), 7.67 (d with fine splitting, J=7.7 Hz, 1H), 7.32 (dd, J=4.8, 7.7 Hz, 1H), 5.47 (d, J=5.5 Hz, 1H, disappears on exchange with D₂O), 4.47 (t, J=5.6 Hz, 1H, disappears on exchange with D₂O), 4.30 (m, 2H), 3.78 (d, 5.6 Hz, 1H), 3.31 (dd, J=5.8, 10.4 Hz, 1H), 3.17 (dd, J=5.8, 10.4 Hz, 1H), 0.80 (s, 3H), 0.79 (s, 3H); ¹³C-NMR (CD₃OD) δ (ppm)=176.4, 149.7, 148.8, 137.8, 137.0, 125.2, 77.5, 70.4, 41.2, 40.6, 21.5, 21.0; Anal. calcd for C₁₂H₁₈N₂O₃: C, 60.49; H, 7.61; N, 11.76, found: C, 60.41; H, 7.57; N, 11.70.

### 6.2. Example

Effects of an Illustrative Compound of the Pathway on Obese Female Zucker Rats

In a number of different experiments, compounds described in Table 1 were administered daily to 11-13 week old chow fed obese female Zucker rats for 14 days in the morning by oral gavage in 20% ethanol/80% polyethylene glycol-200 (dosing vehicle)(“EP”). The dosing vehicle was administered to control animals in parallel experiments.

Body weight was determined daily prior to dosing. Animals were allowed free access to rodent chow and water throughout the study. Blood glucose was determined after a 6-hour fast in the afternoon without anesthesia from a tail vein. Serum was also prepared from a blood sample subsequently obtained from the orbital venous plexus (with O₂/CO₂ anesthesia) prior to and after one week treatment and used lipid and insulin determinations. At two weeks, blood glucose was again determined after a 6-hour fast without anesthesia from a tail vein. Soon thereafter, animals were sacrificed by CO₂ inhalation in the afternoon and cardiac blood serum was collected and assessed for various lipids and insulin.

Generally, illustrative compounds improved the ratio of non-HDL cholesterol to HDL cholesterol content relative to control, and generally illustrative compounds reduced serum triglyceride content.

Illustrative compounds reduced serum levels of harmful triglycerides, reduced serum levels of harmful non-esterified fatty acids, and elevated levels of the beneficial β-hydroxy butyrate.

TABLE 1
Examples of effects of oral daily treatment of obese female Zucker rats with
compounds of the invention for fourteen days (n is number of animals per experiment)

| | | | | | | Percent Change from Pre-treatment | | | | | | | |
|---|---|---|---|---|---|---|---|---|---|---|---|---|---|
| | | | Dose. | % | | | | | | | | | |
| | | | (mg/kg/ | wt. | HDL-C/ | | | Non | | | | | |
| Compd | Expt. # | n | day) | gain | non HDL-C | TG | TC | HDL-C | HDL-C | Glucose | Insulin | NEFA | BHA |
| Vehicle | LR88 | 5 | — | 10 | 2 | −8 | −2 | 38 | −23 | 1 | 2 | 18 | 60 |
| AA | LR88 | 3 | 100 | 11 | 3 | −38 | 41 | −17 | 87 | 10 | −10 | −30 | 51 |
| Vehicle | LR90 | 4 | — | 12 | 1 | 27 | 0 | 15 | −11 | 7 | 26 | 79 | 9 |
| W | LR90 | 4 | 92 | 10 | 1 | −1 | −18 | −20 | −10 | 5 | 15 | 60 | −12 |
| Vehicle | LR83 | 4 | — | 8 | 1 | 52 | 57 | 135 | −10 | −7 | −11 | 41 | 3 |
| V1 | LR83 | 2 | 100 | 11 | 1 | 22 | 34 | 6 | 77 | −3 | −20 | 7 | 63 |
| Vehicle | LR54 | 4 | — | 13 | 1 | 52 | −10 | 32 | −34 | 8 | −31 | 17 | 95 |
| V2 | LR54 | 3 | 30 | 10 | 2 | 36 | −13 | 23 | −20 | −8 | −53 | −23 | 58 |
| Vehicle | LR45 | 4 | — | 9 | 2 | 44 | 7 | 25 | −1 | −22 | −29 | 14 | 77 |
| U | LR45 | 4 | 100 | 9 | 2 | 1 | 8 | −2 | 14 | −3 | 6 | 13 | 254 |
| Vehicle | LR65 | 4 | — | 11 | 2 | 19 | 2 | 76 | −18 | −7 | 2 | −16 | 107 |
| V3 | LR65 | 5 | 30 | 9 | 2 | 14 | 9 | 16 | 7 | 3 | 11 | −16 | 69 |
| Vehicle | LR65 | 4 | — | 11 | 2 | 19 | 2 | 76 | −18 | −7 | 2 | −16 | 107 |
| V4 | LR65 | 5 | 30 | 12 | 1 | 22 | 23 | 75 | 5 | 2 | 4 | −28 | 79 |

Accordingly, the compounds of the present invention or pharmaceutically acceptable salts, solvates, hydrates, clathrates, or prodrugs thereof, are useful for improving the ratio of HDL:non-HDL cholesterol in the blood, reducing serum triglycerides, and/or elevating HDL-cholesterol, without the adverse side effect of promoting weight gain in a patient to whom the compound is administered.

### 6.3. Example

Effect of an Illustrative Compound of the Invention on the Synthesis of Total Lipids in Hepatocytes Isolated from a Male Sprague-Dawley Rat

A male Sprague-Dawley rate was anesthetized by administration of sodium pentobarbitol by intraperitoneal at 80 mg/kg. In situ perfusion of the liver was performed as follows. The abdomen of the animal was opened, the portal vein canulated, and the liver perfused with WOSH solution (149 mM NaCl, 9.2 mM Na HEPES, 1.7 mM Fructose, 0.5 mM EGTA, 0.029 mM Phenol red, 10 U/ml heparin, pH 7.5) at a flow rate of 30 ml/min for 6 minutes. To digest the liver, DSC solution (6.7 mM KCl, 143 mM NaCl, 9.2 mM Na HEPES, 5 mM CaCl₂-2H₂O, 1.7 mM Fructose, 0.029 mM Phenol red, 0.2% BSA, 100 U/ml collagenase Type I, 160 BAEE/ml trypsin inhibitor, pH 7.5) was perfused through the liver at a flow rate of 30 ml/min for 6 minutes at a temperature of 37° C. After digestion, cells were dispersed in a solution of DMEM-(DMEM containing 2 mM GlutMax-1, 0.2% BSA, 5% FBS, 12 nM insulin, 1.2 μM hydrocortisone) to stop the digestion process. The crude cell suspension was filtered through three layers of stainless steel mesh with pore sizes of 250, 106, and 75 μm respectively. Filtered cells were centrifuged at 50×g for two minutes and the supernatant discarded. The resulting cell pellet was resuspended in DMEM and centrifuged again. This final cell pellet was resuspended in DMEM+HS solution (DMEM containing 2 mM GlutMax-1, 20 mM delta-aminolevulinic acid, 17.4 mM MEM non-essential amino acids, 20% FBS, 12 nM insulin, 1.2 μM hydrocortisone) and plated to form monolayer cultures at a density of 100×10³ cells/cm² on collagen coated culture dishes. Four hours after initial plating, media was changed to DMEM+ (DMEM containing 2 mM GlutMax-1, 20 nM delta-aminolevulinic acid, 17.4 mM MEM non-essential amino acids, 10% FBS, 12 nM insulin, 1.2 μM hydrocortisone) and remained on cells overnight.

To test the effect of an illustrative compound of the invention on synthesis rates of total lipids, the monolayer cultures were exposed to 1, 3, 10, 30, 100, or 300 μM of Compound AC in DMEM+ containing 1 μCi/ml ¹⁴C-acetate, D-glucose, hepes, glutamine, lucine, alanine, lactate, pyruvate, non-essential amino acids, BSA, insulin, and gentamicin. Control cells were exposed to the same media lacking lovastatin or the test compounds. All cells were exposed to 0.1% DMSO. Metabolic labeling with ¹⁴C-acetate continued for 4 hr at 37° C. After labeling, cells were washed twice with 1 mL of PBS followed by addition of scintillant (Microsecent E) and counted on a Topcount.® The IC₅₀ value is indicated in Table 2 and shows reduction in total lipid synthesis in primary rat hepatocytes.

TABLE 2
Example of IC₅₀

| Compound | IC₅₀ (μm) |
|---|---|
| 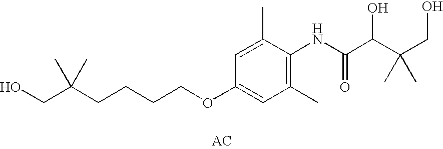 | 2.1 |

Accordingly, the compounds of the present invention, in which Compound AC or a pharmaceutically acceptable salt, solvate, hydrate, clathrate, or prodrug thereof is illustrative, are useful for reducing lipid synthesis in a patient.

The present invention is not to be limited in scope by the specific embodiments disclosed in the examples which are intended as illustrations of a few aspects of the invention and any embodiments which are functionally equivalent are within the scope of this invention. Indeed, various modifications of the invention in addition to those shown and described herein will become apparent to those skilled in the art and are intended to fall within the appended claims.

What is claimed is:

1. A compound of formula I: 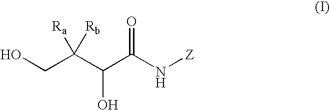
or pharmaceutically acceptable salts, solvates, clathrates, hydrates, or prodrugs thereof, wherein:
Z is (C₆-C₁₄)aryl, (C₁-C₆)alkyl, cylcoalkyl, heteroaryl, cycloheteroalkyl, or —(CH₂)ₙ—X—(CH₂)ₙ—Y;
X is O, S, Se, C(O), C(H)F, CF₂, S(O), NH, O—P(O)(OH)—O, NH—C(O)—NH or NH—C(S)—NH;
Y is —COOH, COO—{(C₁-C₆)alkyl}, COO—{(C₆-C₁₄)aryl}, —COO-(cycloalkyl), —COO-(heteroaryl). —COO-(heterocycloalkyl), —OH, —OPO₃H, —OP₂O₆H₂, —OPO₃-(nucleotide), —OP₂O₆(H)-(nucleotide), or 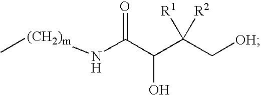
either (a) R¹ is hydrogen, methyl, or phenyl; and R² is methyl or phenyl; or (b) R¹ and R² are aken together to form a cycloalkyl ring of 3 to 6 carbons;
n and m are independently an integer from 0 to 6.

2. A compound of formula II: 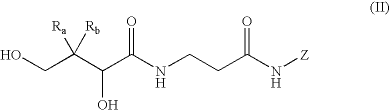
or pharmaceutically acceptable salts, solvates, clathrates, hydrates, or prodrugs thereof, wherein:
Z is (C₆-C₁₄)aryl, (C₁-C₆)alkyl, cylcoalkyl, heteroaryl, cycloheteroalkyl, or —(CH₂)ₙ—X—(CH₂)ₙ—Y;
X is O, S, Se, C(O), C(H)F, CF₂, S(O), NH, O—P(O)(OH)—O, NH—C(O)—NH or NH—C(S)—NH;
Y is —COOH, COO—{(C₁-C₆)alkyl}, COO—{(C₆-C₁₄)aryl}, —COO-(cycloalkyl), —COO-(heteroaryl). —COO-(heterocycloalkyl), —OH, —OPO₃H, —OP₂O₆H₂, —OPO₃-(nucleotide), —OP₂O₆(H)-(nucleotide), or 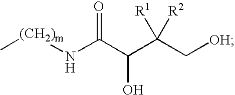
either (a) R¹ is hydrogen, methyl, or phenyl; and R² is methyl or phenyl; or (b) R¹ and R² are taken together to form a cycloalkyl ring of 3 to 6 carbons; and
m is an integer from 0 to 6.

3. A compound of formula III: 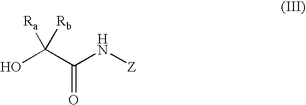
or pharmaceutically acceptable salts, solvates, clathrates, hydrates, or prodrugs thereof, wherein:
Z is (C₆-C₁₄)aryl, (C₁-C₆)alkyl, cylcoalkyl, heteroaryl, cycloheteroalkyl, or —(CH₂)ₙ—X—(CH₂)ₙ—Y;
X is O, S, Se, C(O), C(H)F, CF₂, S(O), NH, O—P(O)(OH)—O, NH—C(O)—NH or NH—C(S)—NH;
Y is —COOH, COO—{(C₁-C₆)alkyl}, COO—{(C₆-C₁₄)aryl}, —COO-(cycloalkyl), —COO-(heteroaryl). —COO-(heterocycloalkyl), —OH, —OPO₃H, —OP₂O₆H₂, —OPO₃-(nucleotide), —OP₂O₆(H)-(nucleotide), or 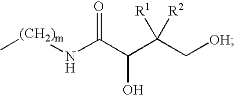
either (a) R¹ is hydrogen, methyl, or phenyl; and R² is methyl or phenyl; or (b) R¹ and R² are taken together to form a cycloalkyl ring of 3 to 6 carbons;
m is an integer from 0 to 6.

4. A composition comprising a compound of claim 1 and a pharmaceutically acceptable vehicle, excipient, or diluent.

5. A composition comprising a compound of claim 2 and a pharmaceutically acceptable vehicle, excipient, or diluent.

6. A composition comprising a compound of claim 3 and a pharmaceutically acceptable vehicle, excipient, or diluent.

7. A method for treating or preventing cardiovascular disease, dyslipidemia, dyslipoproteinemia, a disorder of glucose metabolism, hypertension, or impotence in a patient, comprising administering to a patient in need of such treatment or prevention a therapeutically or prophylactically effective amount of a compound of claim 1.

8. A method for treating or preventing Alzheimer's Disease, Syndrome X, a peroxisome proliferator activated receptor-associated disorder, septicemia, a thrombotic disorder, obesity, pancreatitis, renal disease, cancer, inflammation, or bacterial infection in a patient, comprising administering to a patient in need of such treatment or prevention a therapeutically or prophylactically effective amount of a compound of claim 1.

9. A method for treating or preventing cardiovascular disease, dyslipidemia, dyslipoproteinemia, a disorder of glucose metabolism, hypertension, or impotence in a patient, comprising administering to a patient in need of such treatment or prevention a therapeutically or prophylactically effective amount of a compound of claim 2.

10. A method for treating or preventing Alzheimer's Disease, Syndrome X, a peroxisome proliferator activated receptor-associated disorder, septicemia, a thrombotic disorder, obesity, pancreatitis, renal disease, cancer, inflammation, or bacterial infection in a patient, comprising administering to a patient in need of such treatment or prevention a therapeutically or prophylactically effective amount of a compound of claim 2.

11. A method for treating or preventing cardiovascular disease, dyslipidemia, dyslipoproteinemia, a disorder of glucose metabolism, hypertension, or impotence in a patient, comprising administering to a patient in need of such treatment or prevention a therapeutically or prophylactically effective amount of a compound of claim 3.

12. A method for treating or preventing Alzheimer's Disease, Syndrome X, a peroxisome proliferator activated receptor-associated disorder, septicemia, a thrombotic disorder, obesity, pancreatitis, renal disease, cancer, inflammation, or bacterial infection in a patient, comprising administering to a patient in need of such treatment or prevention a therapeutically or prophylactically effective amount of a compound of claim 3.

13. A method for treating or preventing a cardiovascular disease in a patient, comprising administering to a patient in need of such treatment or prevention a therapeutically or prophylactically effective amount of a compound of claim 1.

14. A method for treating or preventing a cardiovascular disease in a patient, comprising administering to a patient in need of such treatment or prevention a therapeutically or prophylactically effective amount of a compound of claim 2.

15. A method for treating or preventing a cardiovascular disease in a patient, comprising administering to a patient in need of such treatment or prevention a therapeutically or prophylactically effective amount of a compound of claim 3.

16. A method for treating or preventing a dyslipidemia in a patient, comprising administering to a patient in need thereof a therapeutically or prophylactically effective amount of a compound of claim 1.

17. A method for treating or preventing a dyslipidemia in a patient, comprising administering to a patient in need thereof a therapeutically or prophylactically effective amount of a compound of claim 2.

18. A method for treating or preventing a dyslipidemia in a patient, comprising administering to a patient in need thereof a therapeutically or prophylactically effective amount of a compound of claim 3.

19. A method for treating or preventing hypertension in a patient, comprising administering to a patient in need thereof a therapeutically or prophylactically effective amount of a compound of claim 1.

20. A method for treating or preventing hypertension in a patient, comprising administering to a patient in need thereof a therapeutically or prophylactically effective amount of a compound of claim 2.

21. A method for treating or preventing hypertension in a patient, comprising administering to a patient in need thereof a therapeutically or prophylactically effective amount of a compound of claim 3.

22. A method for treating or preventing cancer in a patient, comprising administering to a patient in need thereof a therapeutically or prophylactically effective amount of a compound claim 1.

23. A method for treating or preventing cancer in a patient, comprising administering to a patient in need thereof a therapeutically or prophylactically effective amount of a compound claim 2.

24. A method for treating or preventing cancer in a patient, comprising administering to a patient in need thereof a therapeutically or prophylactically effective amount of a compound claim 3.

25. A method for treating or preventing inflammation in a patient, comprising administering to a patient in need thereof a therapeutically or prophylactically effective amount of a compound of claim 1.

26. A method for treating or preventing inflammation in a patient, comprising administering to a patient in need thereof a therapeutically or prophylactically effective amount of a compound of claim 2.

27. A method for treating or preventing inflammation in a patient, comprising administering to a patient in need thereof a therapeutically or prophylactically effective amount of a compound of claim 3.

28. A method for treating or preventing impotence in a patient, comprising administering to a patient in need thereof a therapeutically or prophylactically effective amount of a compound of claim 1.

29. A method for treating or preventing impotence in a patient, comprising administering to a patient in need thereof a therapeutically or prophylactically effective amount of a compound of claim 2.

30. A method for treating or preventing impotence in a patient, comprising administering to a patient in need thereof a therapeutically or prophylacticall effective amount of a compound of claim 3.

31. A single unit dosage form comprising a compound of claim 1 in an amount from about 0.001 mg to about 200 mg.

32. The dosage form of claim 31, wherein the amount is from about 0.025 mg to about 150 mg.

33. The dosage form of claim 32, wherein the amount is from about 0.05 mg to about 100 mg.

34. The dosage form of claim 31 which is formulated for oral administration.

35. The dosage form of claim 34 which is a solid.

36. The dosage form of claim 31 which is formulated for parenteral administration.

37. The dosage form of claim 36, wherein the dosage form is a sterile solution.

38. The dosage form of claim 31, wherein the dosage form is suitable for mucosal or transdermal administration.

39. A single unit dosage form comprising a compound of claim 2 in an amount from about 0.001 mg to about 200 mg.

40. The dosage form of claim 39, wherein the amount is from about 0.025 mg to about 150 mg.

41. The dosage form of claim 39, wherein the amount is from about 0.05 mg to about 100 mg.

42. The dosage form of claim 39, which is formulated for oral administration.

43. The dosage form of claim 42 which is a solid.

44. The dosage form of claim 39, which is formulated for parenteral administration.

45. The dosage form of claim 44, wherein the dosage form is a sterile solution.

46. The dosage form of claim 39, wherein the dosage form is suitable for mucosal or transdermal administration.

47. A single unit dosage form comprising a compound of claim 3 in an amount from about 0.001 mg to about 200 mg.

48. The dosage form of claim 47, wherein the amount is from about 0.025 mg to about 150 mg.

49. The dosage form of claim 47, wherein the amount is from about 0.05 mg to about 100 mg.

50. The dosage form of claim 47, which is formulated for oral administration.

51. The dosage form of claim 50, which is a solid.

52. The dosage form of claim 47, which is formulated for parenteral administration.

53. The dosage form of claim 52, wherein the dosage form is a sterile solution.

54. The dosage form of claim 47, wherein the dosage form is suitable for mucosal or transdermal administration.

55. A method for identifying a compound useful for treating or preventing a condition in a patient comprising:
a) docking a three-dimensional structure of a test compound with a three-dimensional structure of a substrate binding site of a short-chain acyl-coenzyme A ligase and determining a first binding energy value therefor;
b) docking the three-dimensional structure of the test compound with a three-dimensional structure of a substrate binding site of a long-chain acyl-coenzyme A ligase and determining a second binding energy value therefor; and
c) determining whether the ratio of the first binding energy value and the second binding energy value.

56. The method of claim 55, wherein the short-chain acyl-coenzyme A ligase is a short chain acyl coenzyme A synthetase or butyrate-CoA ligase.

57. The method of claim 55, wherein the long-chain acyl-coenzyme A ligase is selected from the group consisting of fatty acyl CoA synthetase and palymitoyl CoA synthetase.

58. The method of claim 55, wherein the ratio is at least 2.

59. The method of claim 55, wherein the ratio is at least 10.

60. The method of claim 55, wherein the ratio is at least 100.

61. The method of claim 7, wherein the amount of compound of claim 1 is from about 0.001 mg to about 200 mg per kilogram body weigh.

62. The method of claim 8, wherein the amount of compound of claim 1 is from about 0.001 mg to about 200 mg per kilogram body weigh.

63. The method of claim 9, wherein the amount of compound of claim 1 is from about 0.001 mg to about 200 mg per kilogram body weigh.

64. The method of claim 10, wherein the amount of compound of claim 1 is from about 0.001 mg to about 200 mg per kilogram body weigh.

65. The method of claim 11, wherein the amount of compound of claim 1 is from about 0.001 mg to about 200 mg per kilogram body weigh.

66. The method of claim 12, wherein the amount of compound of claim 1 is from about 0.001 mg to about 200 mg per kilogram body weigh
